(19) United States
(12) Patent Application Publication
(10) Pub. No.: US 2016/0317503 A1
(43) Pub. Date: Nov. 3, 2016

(54) ZOLMITRIPTAN POWDERS FOR PULMONARY DELIVERY

(71) Applicant: Civitas Therapeutics, Inc., Chelsea, MA (US)

(72) Inventor: Michael M. Lipp, Framingham, MA (US)

(21) Appl. No.: 15/143,131

(22) Filed: Apr. 29, 2016

Related U.S. Application Data
(60) Provisional application No. 62/155,910, filed on May 1, 2015.

(51) Int. Cl. A61K 31/422; A61K 9/00; A61K 9/16

(52) U.S. Cl. CPC A61K 31/422; A61K 9/1617; A61K 9/0075; A61K 9/1652

(57) ABSTRACT

The invention provides powder formulations containing zolmitriptan, or a pharmaceutically acceptable salt thereof, which are useful for pulmonary administration to the respiratory tract of a patient for the treatment of disease.

### RELATED APPLICATION

This application claims the benefit of U.S. Provisional Application No. 62/155,910, filed May 1, 2015. The entire teachings of the above application are incorporated herein by reference.

### BACKGROUND OF THE INVENTION

Zolmitriptan, which has the structure below, is a drug used in the treatment of migraine.

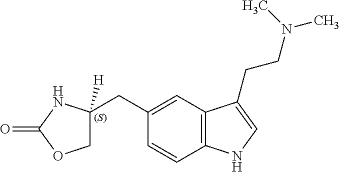

Zolmitriptan is available in the form of tablets for oral administration and a solution for nasal spray. Available formulations of zolmitriptans and other triptans have certain significant disadvantages. Oral zolmitriptan formulations can display a slow and variable onset of action. Oral zolmitriptan formulations are also problematic to administer to patients experiencing nausea as a symptom of their migraine and can also induce nausea and vomiting on their own, limiting their effectiveness. Fast dissolving oral and nasal zolmitriptan formulations potentially possess improved tolerability and rapid onset of action, but have an unpleasant bitter taste which is exacerbated by the relatively high effective doses.

Development of new triptan formulations which overcome these disadvantages is hampered by the low melting points and corresponding low glass transition temperatures of the triptan class, which make it difficult to obtain stable amorphous triptan powders, as well as the difficulty in ameliorating the bitter taste typically associated with these compounds.

There is a need for new zolmitriptan formulations and methods of delivery thereof which overcome the disadvantages of the available formulations.

### SUMMARY OF THE INVENTION

The present invention provides stable, spray-dried powder formulations containing zolmitriptan or a pharmaceutically acceptable salt of zolmitriptan, which are useful for administration to the respiratory tract for treating migraine and cluster headaches.

In an embodiment, the invention relates to a dry powder formulation of zolmitriptan having a fine particle fraction less than 5.6 microns (“FPF<5.6”) of at least about 60%.

In an embodiment, the invention relates to a dry powder formulation of zolmitriptan produced by spray drying.

In an embodiment, the invention relates to a dry powder formulation of zolmitriptan which is between about 5 and about 50% zolmitriptan by weight.

In an embodiment, the invention relates to a dry powder formulation of zolmitriptan which is between about 5 and about 30% zolmitriptan by weight.

In an embodiment, the invention relates to a dry powder formulation comprising zolmitriptan or a pharmaceutically acceptable salt thereof, a phospholipid, a salt and an additional excipient which is an amino acid, a sugar or a sugar alcohol. Preferably, the powder formulation has a FPF<5.6 which is greater than about 60%. More preferably, the powder formulation also has a fine particle fraction less than 3.4 microns (“FPF<3.4”) which is greater than about 30%.

In an embodiment, the invention relates to a dry powder formulation comprising zolmitriptan, dipalmitoylphosphatidylcholine (DPPC), sodium chloride or sodium citrate and L-leucine or polyglycitol.

In an embodiment, the invention relates to a dry powder formulation comprising zolmitriptan, dipalmitoylphosphatidylcholine (DPPC), sodium chloride, and L-leucine.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic of the NIRO PSD-1 spray drying apparatus used for powder production as described in the Examples.

FIG. 2 displays modulated differential scanning calorimetry data for a 100% zolmitriptan amorphous spray-dried powder.

FIG. 3A displays representative XRPD data for crystalline zolmitriptan starting material.

FIG. 3B displays XRPD data for spray-dried 100% zolmitriptan as described in Example 3.

FIG. 4A provides XRPD characterization data for zolmitriptan formulation 155137 described in Example 3.

FIG. 4B provides XRPD characterization data for zolmitriptan formulation 155140 described in Example 3.

FIG. 4C provides XRPD characterization data for zolmitriptan formulation 155145 described in Example 3.

FIG. 5 displays representative XRPD characterization data for Formulation A.

FIG. 6 is a schematic of the tumbling apparatus used for conditioning filled zolmitriptan powder capsules.

FIG. 7A presents pharmacokinetic results for the Zomig™ 5 mg oral dosing group.

FIG. 7B presents pharmacokinetic results for the Zomig™ 5 mg intranasal dosing group.

FIG. 7C presents pharmacokinetic results for the Formulation A 0.6 mg dosing group.

FIG. 7D presents pharmacokinetic results for the Formulation A 1.2 mg dosing group.

FIG. 7E presents pharmacokinetic results for the Formulation A 2.4 mg dosing group.

FIG. 7F presents pharmacokinetic results for the Formulation A 4.8 mg dosing group.

### DETAILED DESCRIPTION OF THE INVENTION

The present invention provides new formulations of zolmitriptan for pulmonary delivery which have reduced unpleasant taste and overcome the poor amorphous phase physical and chemical instability of this class of agents. In particular, Applicants have found that inhalation of zolmitriptan free base powder formulation having a relatively high FPF<5.6, for example an FPF<5.6 of about 60% or higher, results in a significantly decreased sensation of unpleasant taste compared to nasal formulations. In addition, formulations of the invention exhibit suitable stability in accelerated stability testing.

In one embodiment, the invention is a zolmitriptan composition formulated for pulmonary delivery comprising a powder, prepared, for example, by spray-drying, and comprising zolmitriptan or a pharmaceutically acceptable salt thereof. The compositions preferably comprise zolmitriptan free base. The zolmitriptan is preferably present in the powder in an amount of about 5% to 50% by weight, preferably from about 5% to about 30% and more preferably about 10% to about 20% by weight of dry solids. In one embodiment, the zolmitriptan is present in the powder in substantially amorphous form. In another embodiment, the additional excipients are also present in substantially amorphous form. Conversely, in another embodiment, one or more of the additional excipients are present in a partially crystalline or substantially crystalline form.

In certain embodiments, the powder particles comprise zolmitriptan, a phospholipid, an optional salt, an optional sugar or sugar alcohol and an optional amino acid. In one embodiment, the particles comprise zolmitriptan or a pharmaceutically acceptable salt thereof, a phospholipid and a salt. In another aspect of this invention, the particles comprise zolmitriptan or a pharmaceutically acceptable salt thereof, a phospholipid, a salt, an optional amino acid, and/or an optional sugar and/or sugar alcohol.

Examples of phospholipids suitable for use in the particles and powders of the invention include, but are not limited to, dipalmitoylphosphatidylcholine (DPPC), dilauroylphosphatidylcholine (DLPC), dimyristoylphosphatidylchloline (DMPC) and distearoyl-phosphatidylcholine (DSPC). Preferred phospholipids include DPPC, DMPC and DSPC. A most preferred phospholipid is DPPC.

The salts suitable for use in powders and particles of the invention include alkali metal salts and alkaline earth metal salts. Examples of suitable salts include, but are not limited to, sodium and potassium salts, such as sodium chloride (NaCl), sodium citrate, sodium lactate, and potassium chloride. Other suitable salts include calcium, magnesium and zinc salts, such as calcium chloride, magnesium chloride or zinc chloride. A preferred salt is sodium chloride.

Examples of amino acids suitable for use in the particles and powders of the invention include, but are not limited to, hydrophobic amino acids, such as leucine, isoleucine, alanine, valine, phenylalanine and glycine. In one embodiment, the amino acid is an L-amino acid or glycine. A preferred amino acid is L-leucine. The terms leucine and L-leucine are used interchangeably herein; both refer to the L enantiomeric form of leucine).

Examples of sugars and sugar alcohols suitable for use in the particles and powders of the invention include, but are not limited to, maltodextrin, polyglycitol, lactose, trehalose and mannitol. Preferred sugars and sugar alcohols are maltodextrin and polyglycitol. In certain embodiments, the maltodextrin has a dextrose equivalence (DE) of 3 to 20%. In certain embodiments, the maltodextrin has a DE of 4-7%, 10-12% or 16-19%.

In one embodiment, the particles comprise zolmitriptan, DPPC, sodium chloride and maltodextrin.

In one embodiment, the particles comprise zolmitriptan, DPPC, sodium chloride and L-leucine.

In one embodiment, the particles comprise zolmitriptan, DPPC, sodium chloride and polyglycitol.

In one embodiment, the particles comprise zolmitriptan, DPPC, sodium chloride, L-leucine and polyglycitol.

In one embodiment, the particles comprise about 5 to about 50% zolmitriptan, about 5 to about 20% phospholipid, and about 1 to about 10% salt as measured by weight percent of dry solids in the powder. In another embodiment, the particles comprise about 5 to about 30% zolmitriptan, about 5 to about 20% phospholipid, and about 1 to about 10% salt as measured by weight percent of dry solids in the powder. In another embodiment, the particles comprise about 10 to about 25% zolmitriptan, or about 15% zolmitriptan, about 5 to about 20% phospholipid, and about 1 to about 10% salt as measured by weight percent of dry solids in the powder. The particles preferably further comprise an excipient, such as a sugar, a sugar alcohol or an amino acid, preferably maltodextrin, L-leucine or polyglycitol, in an amount from about 50% to about 80% as measured by weight percent of dry solids. In one embodiment, the particles comprise by dry weight about 15% zolmitriptan, about 2% salt, about 18% phospholipid and about 65% sugar, sugar alcohol or amino acid. In one embodiment the powder is selected from the formulations shown in Table 1, where the amount of each component is provided as weight %.

TABLE 1

| Zolmi- | | Sodium | Maltodextrin | L- | |
|---|---|---|---|---|---|
| triptan | DPPC | Chloride | (DE = 10.7%) | Leucine | Polyglycitol |
| 25% | 18% | 2% | 55% | 0 | 0 |
| 10% | 18% | 2% | 70% | 0 | 0 |
| 10% | 18% | 2% | 0 | 0 | 70% |
| 10% | 8% | 2% | 0 | 0 | 80% |
| 20% | 8% | 2% | 0 | 0 | 70% |
| 20% | 18% | 2% | 0 | 0 | 60% |
| 10% | 18% | 2% | 0 | 70% | 0 |
| 10% | 8% | 2% | 0 | 80% | 0 |
| 20% | 8% | 2% | 0 | 70% | 0 |
| 20% | 18% | 2% | 0 | 60% | 0 |
| 15% | 18% | 2% | 0 | 0 | 65% |
| 15% | 18% | 2% | 0 | 65% | 0 |

In certain embodiments, the powder particles comprise about 5 to about 50% zolmitriptan, about 5 to about 20% DPPC, about 1 to about 10% sodium chloride and about 50 to about 80% L-leucine, as measured by weight percent of dry solids in the powder. In another embodiment, the particles comprise about 10 to about 25% zolmitriptan, about 5 to about 20% DPPC, about 1 to about 10% sodium chloride and about 55 to 75% L-leucine as measured by weight percent of dry solids in the powder. In another embodiment, the particles comprise about 10 to about 20% zolmitriptan, about 15 to about 20% DPPC, about 1 to about 5% sodium chloride and about 60 to 70% L-leucine as measured by weight percent of dry solids in the powder.

In one embodiment, the composition of the powder formulation is 15% zolmitriptan, 18% DPPC, 2% sodium chloride and 65% L-leucine by weight of dry solids. This composition is also referred to herein as Formulation A.

In one embodiment, the powders of the invention have a tap density of less than about 0.4 g/cm³. For example, the powders have a tap density between 0.02 and 0.20 g/cm³, between 0.02 and 0.15 g/cm³, between 0.03 and 0.12 g/cm³, between 0.05 and 0.15 g/cm³, or less than about 0.15 g/cm³, or a tap density less than about 0.10 g/cm³, a tap density less than about 0.15 g/cm³. In one embodiment, the powders of the invention have a tap density of less than about 0.2 g/cm³. In another aspect of the invention, the tap density is from about 0.02 to 0.175 g/cm³. In a further aspect of the invention, the tap density is from about 0.06 to 0.175 g/cm³.

Tap density can be measured by using instruments known to those skilled in the art such as the Dual Platform Microprocessor Controlled Tap Density Tester (Vankel, N.C.) or a GEOPYC™ instrument (Micrometrics Instrument Corp., Norcross, Ga., 30093). Tap density is a standard measure of the envelope mass density. Tap density can be determined using the method of USP Bulk Density and Tapped Density, United States Pharmacopia convention, Rockville, Md., 10th Supplement, 4950-4951, 1999. Features which can contribute to low tap density include irregular surface texture and porous structure. The envelope mass density of an isotropic particle is defined as the mass of the particle divided by the minimum sphere envelope volume within which it can be enclosed. In one embodiment of the invention, the particles have an envelope mass density of less than about 0.4 g/cm³.

The inhalable powder of the invention has a preferred particle size, e.g., a volume median geometric diameter (VMGD) of at least about 1 micron (μm). In embodiments, the VMGD is greater than 3 μm or greater than 5 μm. In other embodiments, the VMGD is between about 5 μm and 20 μm, between about 5 μm and 10 μm, between about 6 μm and 15 μm and between about 7 μm and 12 μm. In another aspect of the invention the powder particles have a volume mean geometric diameter of about 2 μm to 15 μm, 3 μm to 12 μm, 3 μm to 8 μm, 5 μm to 9 μm, or 6 μm to 9 μm. The diameter of the spray-dried particles, for example, the VMGD, can be measured using a laser diffraction instrument (for example Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition to targeted sites within the respiratory tract.

The particles of the inhalable powder of the invention preferably have a “mass median aerodynamic diameter” (MMAD), also referred to herein as “aerodynamic diameter”, between about 1 μm and about 5 μm or any subrange encompassed between about 1 μm and about 5 μm. For example, but not limited to, the MMAD is between about 1 μm and about 3 μm, or the MMAD is between about 3 μm and about 5 μm. In one embodiment, the MMAD is between 1.5 μm and 2.5 μm. Experimentally, aerodynamic diameter can be determined by employing a gravitational settling method, whereby the time for an ensemble of powder particles to settle a certain distance is used to infer directly the aerodynamic diameter of the particles. An indirect method for measuring the mass median aerodynamic diameter (MMAD) is the multi-stage liquid impinger (MSLI). The aerodynamic diameter, dₐₑᵣ, can be calculated from the equation:

dₐₑᵣ=d_{g}√ρₜₐₚ

where d_{g} is the geometric diameter, for example the MMGD, and ρ is the powder density.

The fine particle fraction less than 5.6 microns, or FPF<5.6 of a powder corresponds to the percentage of particles in the powder that have an aerodynamic diameter of less than 5.6 The FPF<5.6 of a powder of the invention is preferably about 40% or more. In certain embodiments the FPF<5.6 of the powder is at least about 50%, 60% or 70%. In one embodiment the FPF<5.6 is about 30% to about 90%. In one embodiment the FPF<5.6 is about 70% to about 95%. In one embodiment the FPF<5.6 is about 70% to about 90%. In one embodiment the FPF<5.6 is about 70% to about 85% or about 70% to about 80%.

The fine particle fraction less than 3.4 microns, or FPF<3.4, of a powder corresponds to the percentage of particles in the powder that have an aerodynamic diameter of less than 3.4 In one embodiment, the FPF<3.4 of a powder of the invention is about 30% or more. In one embodiment the FPF<3.4 of the powder is at least about 40% or 50%. In one embodiment the FPF<3.4 is about 30% to 60%.

Preferred powders of the invention are those comprising zolmitriptan, DPPC, sodium chloride and L-leucine as described herein, which have the a FPF<5.6 as described above. More preferably, such powders also have an FPF<3.4 as described above. In one embodiment, the invention is a pharmaceutical composition for pulmonary delivery comprising particles of zolmitriptan or a pharmaceutically acceptable salt thereof having a volume median geometric diameter of greater than about 5 μm and a tap density of less than about 0.20 g/cm³.

In one embodiment, particles of this invention have an external surface area of greater than 5 m²/g. In another embodiment, the external surface area is greater than 10 m²/g, greater than 20 m²/g or about 10 to about 50 m²/g.

The powders of the invention can comprise zolmitriptan and excipients in different physical forms. For example, the powders preferably comprise zolmitriptan in amorphous form and at least one excipient in a crystalline or a partially or substantially crystalline form. Such compositions provide high aerosolizability/dispersibility, rapid dissolution of zolmitriptan in the lung environment and long-term physicochemical stability of zolmitriptan in the solid-state.

In one embodiment, the powder comprises zolmitriptan in an amorphous form, which aids rapid dissolution, dispersed in a predominantly crystalline matrix of leucine, with DPPC and sodium chloride also included to further improve powder formation and aerosolization performance.

In another embodiment, the powders of the invention comprise zolmitriptan in an amorphous form in a solid dispersion with polyglycitol along with DPPC in a partially crystalline form and sodium chloride.

Both of the foregoing embodiments comprises unique physicochemical forms of zolmitriptan-containing powders and differ significantly from conventional dry powder forms of drugs for pulmonary administration (i.e., lactose blends, etc.).

It is surprising that powders containing a substantially amorphous active agent, such as zolmitriptan, in combination with a predominantly crystalline excipient, such as leucine, exhibit enhanced dispersibility and aerosolizability properties compared to conventional formulations, yet remain physically and chemically stable over time at ambient and accelerated temperature storage conditions. In particular, the amorphous phase of zolmitriptan in the powders of the invention is resistant to conversion to a crystalline form in the presence of a crystalline excipient Conversion of powder components to a crystalline form over time typically results in a drastic reduction in the aerosolizability of said powders due to sintering and bridging occurring between particles due to the conversion to the crystalline state.

In another embodiment, the invention is a method of delivering zolmitriptan to the pulmonary system of a patient comprising the steps of:

providing a powder of the invention in a compartment and an inhaler to a patient;

dispersing the powder by breath actuation of the patient; and

delivering the powder particles to the patient's respiratory system.

In one aspect of this invention, an inhaler is a dry powder inhaler. A variety of inhalers can be used including the Aerolizer, Diskus, Flexhaler, Handihaler, Neohaler, Pressair, Rotahaler, Turbohaler, and Twisthaler. Other dry powder inhalers which can be used are described in U.S. Pat. No. 6,766,799, U.S. Pat. No. 7,278,425 and U.S. Pat. No. 8,496,002 each of which is hereby incorporated in by reference for its disclosure relating to the inhalation devices described therein. In one aspect of the invention, the compartment is a capsule or a blister pack. In one aspect of the invention, the inhaler has a resistance of about 0.05 to about 0.25, about 0.15 to about 0.25, 0.05 to about 0.15, 0.2 to about 0.25, or about 0.2. Resistance as referred to herein is measured in: Square root of Cm_{H2O}/Liters per minute.

Gravimetric analysis, using Cascade impactors, is a method of measuring the size distribution of airborne particles. The Andersen Cascade Impactor (ACI) is an eight-stage impactor that can separate aerosols into nine distinct fractions based on aerodynamic size. The size cutoffs of each stage are dependent upon the flow rate at which the ACI is operated. Preferably the ACI is calibrated at 60 L/min. In one embodiment, a two-stage collapsed ACI is used for particle optimization. The two-stage collapsed ACI consists of stages 0, 2 and F of the eight-stage ACI and allows for the collection of two separate powder fractions. At each stage an aerosol stream passes through the nozzles and impinges upon the surface. Particles in the aerosol stream with a large enough inertia will impact upon the plate. Smaller particles that do not have enough inertia to impact on the plate will remain in the aerosol stream and be carried to the next stage.

The ACI is calibrated so that the fraction of powder that is collected on a first stage corresponds to FPF<5.6. The fraction of powder that passes the first stage of the ACI and is deposited on the collection filter corresponds to FPF<3.4. The FPF<5.6 fraction has been demonstrated to correlate to the fraction of the powder that is deposited in the lungs of the patient, while the FPF<3.4 fraction has been demonstrated to correlate to the fraction of the powder that reaches the deep lung regions of a patient. In accordance with the invention, the FPF<5.6 of the inhalable powder of the nominal dose contained in the capsule (i.e., the percentage of particles in the powder contained in the capsule that have an aerodynamic diameter of less than 5.6 μm) is preferably about 40% or more. In one embodiment the FPF<5.6 of the nominal dose of the inhalable powder contained in the capsule is at least about 50%, 60% or 70%. In one embodiment the FPF<5.6 is about 30% to about 90% of the nominal dose of the inhalable powder contained in the inhaler. In one embodiment the FPF<5.6 is at least about 70% of the nominal dose of the inhalable powder contained in the inhaler. In one embodiment the FPF<5.6 is about 70% to about 95% of the nominal dose of the inhalable powder contained in the inhaler. In one embodiment the FPF<5.6 is about 70% to about 90% of the nominal dose of the inhalable powder contained in the inhaler. In one embodiment the FPF<5.6 is about 70% to about 85% or about 70% to about 80% of the nominal dose of the inhalable powder contained in the inhaler. In one embodiment, the FPF<3.4 of a powder of the invention is about 30% or more. In one embodiment the FPF<3.4 of the powder is at least about 40% or 50%. In one embodiment the FPF<3.4 is about 30% to 60%.

As used herein, the term “nominal powder dose” is the total amount of powder held in the capsule. As used herein, the term “nominal drug dose” is the total amount of zolmitriptan contained in the nominal powder dose. The nominal powder dose is related to the nominal drug dose by the load percent of drug in the powder. In one embodiment, the nominal powder dose is 2 to 50 mg, preferably 5 to 50 mg or 25 to 50 mg by dry weight. In a further embodiment, the nominal powder dose is 5 to 40 mg, 5 to 25 mg or 25 to 40 mg by dry weight. In a still further embodiment, the nominal powder dose is 30 to 35 mg by dry weight or 32 to 38 mg by dry weight.

Another method for measuring the size distribution of airborne particles is the multi-stage liquid impinger (MSLI). The multi-stage liquid impinger (MSLI) operates on the same principles as the Anderson Cascade Impactor (ACI), but instead of eight stages there are five in the MSLI. Additionally, instead of each stage consisting of a solid plate, each MSLI stage consists of a methanol-wetted glass frit. The wetted stage is used to prevent bouncing and re-entrainment, which can occur using the ACI. The MSLI is used to provide an indication of the flow rate dependence of the powder. This can be accomplished by operating the MSLI at 30, 60, and 90 L/min and measuring the fraction of the powder collected on stage 1 and the collection filter. If the fractions on each stage remain relatively constant across the different flow rates then the powder is considered to be approaching flow rate independence.

Powders of this invention are typically produced by spray drying. In some cases, spray-drying can produce extremely dry particles which may have poor handling properties and may be difficult to compact into a capsule in a dense manner. A nitrogen source with a specified moisture level may be blown over, across, or through the dry powder to add a specific moisture content to the dry powder. Such moisture can provide the desired working density of the powder. Spray drying methods in accordance with the invention are described in the Examples herein and in U.S. Pat. Nos. 6,848,197 and 8,197,845, incorporated herein by reference.

The inhalable powder comprising zolmitriptan as described above is used to fill capsules suitable for use in an inhaler. The term “capsule material” as used herein refers to the material from which the shell of the capsule for inhalation is made. In one embodiment, the capsule material according to the invention is selected from among gelatin, cellulose derivatives, starch, starch derivatives, chitosan and synthetic plastics.

If gelatin is used as the capsule material, examples according to the invention may be selected from among polyethyleneglycol (PEG), PEG 3350, glycerol, sorbitol, propyleneglycol, PEO-PPO block copolymers and other polyalcohols and polyethers. If cellulose derivatives are used as the capsule material, examples according to the invention may be selected from hydroxypropylmethylcellulose (HPMC), hydroxypropylcellulose, methylcellulose, hydroxymethylcellulose and hydroxyethylcellulose. If synthetic plastics are used as the capsule material, examples according to the invention may be selected from polyethylene, polycarbonate, polyester, polypropylene and polyethylene terephthalate. In one embodiment, the capsule material further comprises titanium dioxide. In one preferred embodiment the capsule comprises HPMC and titanium dioxide. In one embodiment, the capsule comprises carrageenan. In a further embodiment, the capsule comprises potassium chloride. In a still further embodiment, the capsule comprises, HPMC, carrageenan, potassium chloride, and titanium dioxide. In one embodiment, the capsule size is selected from 000, 00, 0, 1, or 2. In a specific embodiment, the capsule size is 2 or 00.

In one specific embodiment, the capsule is a hydroxypropylmethylcellulose (HPMC) capsule. In another specific embodiment, the capsule is a HPMC size 2 capsule. In another specific embodiment, the capsule is a HPMC size 00 capsule. In another specific embodiment the capsule material comprises HPMC and titanium dioxide and the capsule size is 2. In another specific embodiment the capsule material comprises HPMC and titanium dioxide and the capsule size is 00.

In one embodiment, a size 2 capsule contains between 4 and 30 mg of the zolmitriptan powder of the invention. In another embodiment, a size 2 capsule contains between 10 and 25 mg of the zolmitriptan powder.

In another embodiment, a size 2 capsule contains between 12 and 20 mg of zolmitriptan powder. In another embodiment, a size 2 capsule contains about 7, 8, 9, 10, 11, 12, 13, 14, 15, 16, 17, 18, 19, 20, 21, 22, 23, 24, 25, 26, 27, 28, 29 or 30 mg of zolmitriptan powder.

In one embodiment, a size 2 capsule contains between 0.5 and 5 mg of zolmitriptan. In another embodiment, a size 2 capsule contains between 0.5 mg and 3.0 mg of zolmitriptan. In another embodiment, a size 2 capsule contains about 0.825, 1.6, 2.4 or 3 mg of zolmitriptan.

In one aspect of the invention, the powders have low electrostatic charge to enable high dispersion from the capsule.

In one embodiment, the zolmitriptan formulations of the invention are prepared and packaged in a manner that prevents or minimizes the degradation of the active ingredient due to oxidation of zolmitriptan. In this embodiment, zolmitriptan powders are produced and packaged in an environment in which the oxygen is minimized or excluded, such as under an atmosphere of an inert gas, such as dry nitrogen or argon. In certain embodiments, an oxygen absorbing/scavenging agent is included in the final packaging in direct communication with the atmosphere surrounding the zolmitriptan powder.

The capsules of the invention are particularly suitable for use in a dry powder inhaler for the delivery of a dry powder composition comprising an effective amount of zolmitriptan to a subject in need thereof, for example, for treating migraine or cluster headache.

In one embodiment, the invention provides a method for treating migraine in a subject in need thereof, preferably a human patient, comprising the step of administering to the subject a therapeutically effective amount of a zolmitriptan composition of the invention. The composition is preferably administered to the subject's pulmonary system, for example, using an inhaler, such as a dry powder inhaler, as described herein. The zolmitriptan compositions of the invention are useful for the acute treatment of migraine, providing relief of one or more symptoms of migraine, for example, pain, nausea, photophobia or phonophobia, and/or shortening the duration of migraine. In certain embodiments, administration of a zolmitriptan composition of the invention provides relief of pain. In certain embodiments, administration of a zolmitriptan composition of the invention provides relief of pain and at least one of nausea, photophobia and phonophobia. In certain embodiments, administration of a zolmitriptan composition of the invention provides relief of at least pain and nausea. In certain embodiments, administration of a zolmitriptan composition of the invention provides relief of each of pain, nausea, photophobia and phonophobia. The migraine to be treated can be migraine with or without aura. In preferred embodiments, administration of the zolmitriptan formulation of the invention provides relief of pain within about 15 minutes or within about 30 minutes of administration.

In another embodiment, the invention provides a method for treating cluster headache in a subject in need thereof, preferably a human patient, comprising the step of administering to the subject a therapeutically effective amount of a zolmitriptan composition of the invention. The zolmitriptan compositions of the invention are useful for acute treatment of cluster headache, that is, providing relief from one or more symptoms of cluster headache and/or shortening the duration of cluster headache.

The composition of the invention is preferably administered to the subject's pulmonary system, for example, using an inhaler, such as a dry powder inhaler, as described herein.

In one embodiment, the subject to be treated is an adult human. In another embodiment, the subject to be treated is a pediatric human, for example a human from about 6 to about 11 years of age, about 12 to about 17 years of age, or about 6 to about 17 years of age.

As used herein, the term “therapeutically effective amount” means the amount needed to achieve the desired effect or efficacy. A therapeutically effective amount of zolmitriptan or a zolmitriptan composition for treating migraine or cluster headache is an amount of zolmitriptan or the zolmitriptan composition which provides relief from migraine or cluster headache or one or more symptoms of migraine or cluster headache. Such an amount of zolmitriptan or zolmitriptan composition thereof preferably shortens the duration and/or intensity of a migraine or cluster headache or one or more symptoms thereof, such as pain and/or aura. The actual effective amount of drug can vary according to the specific drug or combination thereof being utilized, the particular composition formulated, the mode of administration, and the age, weight, condition of the patient, and severity of the episode being treated.

In certain embodiments, the zolmitriptan formulation is administered to the subject two or more times for a single migraine episode or cluster headache. For example a first dose can be administered to the subject, followed by a second or more doses at appropriate time intervals, such as about 1 or 2 hour intervals.

In one embodiment, the dose of zolmitriptan administered to the lungs of the subject is about 1.2 or about 2.4 mg. In certain embodiments the nominal dose administered to the subject is about 1.65 mg or about 3 mg. In certain embodiments, the administration of a nominal dose of about 1.65 mg zolmitriptan results in an about 1.2 mg zolmitriptan dose administered to the lungs of the subject. In certain embodiments, the administration of a nominal dose of about 3 mg zolmitriptan results in an about 2.4 mg zolmitriptan dose administered to the lungs of the subject.

In certain embodiments, the dose administered to the subject will be increased over time. For example, upon initial treatment, the subject may receive a lower dose of zolmitriptan, such as a 1.2 mg dose administered to the lungs, and over time for subsequent migraines or cluster headaches the dose may be increased, for example to a 2.4 mg dose administered to the lungs. In certain embodiments, the zolmitriptan formulations of the invention upon pulmonary delivery provide rapid and efficient delivery of zolmitriptan to the systemic circulation comparable to subcutaneous delivery. In certain embodiments, the zolmitriptan formulations of the invention provide a slower and/or prolonged conversion of zolmitriptan to its active metabolite N-desmethylzolmitriptan in the systemic circulation over time over that typically seen after oral dosing of zolmitriptan. In certain embodiments, the pulmonary administration of an effective dose of a zolmitriptan formulation of the invention provides improved efficacy and/or reduced probability of one or more side effects or adverse events compared to administration of an effective dose of an oral or intranasal formulation. For example, compared to administration of an effective oral or intranasal dosage form, the pulmonary administration of a zolmitriptan formulation of the invention can be associated with a decreased probability of one or more of the following: chest and/or throat, neck and jaw pain/tightness/pressure; other vasospasm reactions; and increased blood pressure. In certain embodiments, the pulmonary administration of a zolmitriptan formulation of the invention can be associated with a decreased probability of myocardial ischemia; myocardial infarction; Prinzmetal Angina; arrthymias and/or serotonin syndrome.

The results of the clinical study described in Example 21, infra, show that pulmonary administration of Formulation A, a zolmitriptan formulation of the invention, results in rapid absorption of zolmitriptan and sustained zolmitriptan levels over several hours. In particular, pulmonary delivery of 2.4 or 4.8 mg zolmitriptan in a composition of the invention yields a tₘₐₓ significantly shorter than the tₘₐₓ for 5 mg zolmitriptan oral and nasal formulations, and Cₘₐₓ and AUC₀₋₂₄ which are significantly greater than those observed for the oral and nasal formulations.

In one embodiment, the invention relates to a powder formulation of zolmitriptan free base which, upon pulmonary administration of a zolmitriptan dose of 0.6, 1.2, 2.4 or 4.8 mg (as delivered to the distal lung) to an adult human subject, exhibits a tₘₐₓ of about 8 to 10 minutes, preferably about 9 minutes or about 10 minutes and a Cₘₐₓ of about 5 to about 40 ng/mL or about 10 to 20 ng/mL. In another embodiment, pulmonary administration of a zolmitriptan dose of 1.2, 2.4 or 4.8 mg (as delivered to the distal lung) to an adult human subject, exhibits a tₘₐₓ of about 8 to 10 minutes and a Cₘₐₓ of about 10 to about 40 ng/mL or about 10 to 20 ng/mL. In certain embodiments, dosing of 2.4 mg (as delivered to the lungs) zolmitriptan results in an AUC₀₋₂₄ for zolmitriptan of about 30 to about 60 ng*hr/mL or preferably about 40 to about 50 ng*hr/mL Such formulations include the zolmitriptan powder formulations described herein, including Formulation A as described in Example 21.

The present invention will be further understood by reference to the following non-limiting examples.

### EXAMPLES

In the tables accompanying the following examples, unless otherwise indicated, the following abbreviations have the indicated meanings:
A-T1: Diffractogram is predominantly amorphous with the exception of one to two peaks in the range of 18-23 2-theta characteristic of DPPC.
A-T2: Diffractogram is predominantly amorphous with the exception of one to two peaks in the range of 18-23 2-theta characteristic of DPPC and one to three peaks in the range of 25 to 35 2-theta characteristic of NaCl.
PC-T2: Diffractogram is partially crystalline with all diffraction peaks associated with leucine and DPPC (no peaks associated with zolmitriptan present).
PC: Diffractogram showed partial crystallinity of zolmitriptan (in addition to peaks characteristic of the excipients).
Tg: Glass transition temperature.
Tm: Melting transition temperature.
Tr: Recrystallization transition temperature.
ΔH: Enthalpy change associated with the corresponding melting or recrystallization transition.
Low T1: Characteristic temperature(s) of the first thermal event(s) observed during a DSC scan (typically corresponding to a Tg).
Low T2: Characteristic temperature(s) of the second set of thermal events observed during a DSC scan (typically corresponding to DPPC phase transitions).
TGA-120: Weight (solvent) loss seen over a thermogravimetric analysis (TA Instruments, Q50 TGA System) scan from 25 to 120° C. (0 (chg) indicates a low solvent content value that was observed to be variable and/or negative due to static charging).
n.c.: not calculated/undetermined.
n.d.: not detected.
NT: not tested.

### Example 1

### Spray Drying of Zolmitriptan Powders

Zolmitriptan powders were prepared using the following method:

### A. Equilibration

1. DPPC and zolmitriptan were allowed to equilibrate to room temperature for at least 30 minutes before weighing.

### B. Weighing and Mixing

1. The required amounts of water and ethanol were weighed and transferred to the aqueous and organic phase feed vessels respectively and the stirring elements in both vessels were turned on.

2. The required amounts of sodium chloride and the excipient of choice (L-leucine or Polyglycitol SD-30) were weighed and added to the aqueous phase vessel and allowed to dissolve without allowing vortex formation.

3. The required amounts of zolmitriptan and DPPC were weighed and added to the organic phase vessel and were allowed to dissolve without vortex formation.

### C. Spray Drying

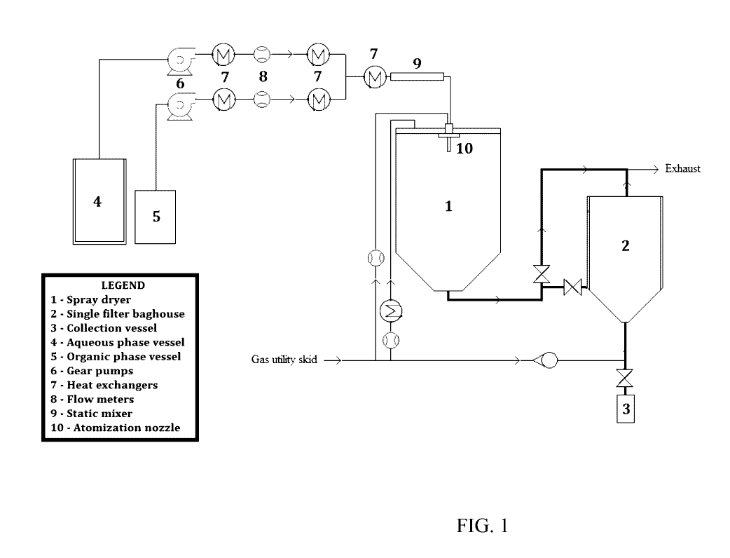

Spray drying was performed using the apparatus set forth in FIG. 1 as follows:

1. Spray drying was initiated by starting the drying gas flow (set to 100 kg/hr) and heating up the drying gas by setting the desired inlet temperature.

2. After the spray dryer outlet temperature reached 75° C., the liquid skid inlet was set to allow blank solvents (Aqueous and Organic at 8 and 32 mL per minute respectively) to be atomized with the aid of nitrogen (atomizing gas flow=22 g/min) into the spray dryer and the system was allowed to cool and stabilize to an outlet temperature of 44° C.

3. Product filter pulsing was initiated and product filter purge flow was set to 15 scfh. After the system stabilized at 44° C., the liquid skid inlet was switched to the feed solvents prepared above and the process was continued until the feed solvents ran out.

4. At the point when feed solvents ran out, the liquid skid inlet was switched back to blank solvents which were allowed to spray for about 10 minutes.

5. At this point, powder collected at the bottom of the product filter was transferred to its final collection vessel in a glove box maintained at an RH of 15%.

6. After spraying the blank solvent for 10 minutes, the system was shut down by shutting down the liquid lines, atomization gas, drying gas heater, drying gas inlet and finally the exhaust.

The results of production runs using this process are set forth in Table 2

TABLE 2

| Lot Number: | | 117187 | 117189 | 155136 |
|---|---|---|---|---|
| SD solution | Total solid concentration (g/L) | 4 | 4 | 4 |
| conc. | | | | |
| Process | Inlet Temperature (° C.) | 78 | 78 | 79 |
| Parameters | Outlet Temperature (° C.) | 44 | 44 | 44 |
| | Drying Gas Rate (kg/hr) | 100 | 100 | 100 |
| | Atomization Gas Flow Rate | 22 | 22 | 22 |
| | (g/min) | | | |
| | Aqueous Flow (mL/min) | 8 | 8 | 8 |
| | Organic flow (mL/min) | 32 | 32 | 32 |
| | Secondary drying gas flow | 0 | 0 | 0 |
| | (kg/hr) | | | |
| | Secondary drying gas | 0 | 0 | 0 |
| | temperature (° C.) | | | |
| | Product filter purge gas flow | 15 | 15 | 15 |
| | (scfh) | | | |
| Solvent | Aqueous phase | Water | Water | Water |
| system | Organic phase | Ethanol | Ethanol | Ethanol |
| Component | Zolmitriptan | 0.25 | 0.1 | 0.1 |
| Fractions | | | | |
| | Polyglycitol SD-30 | | | 0.7 |
| | Maltodextrin (DE = 10.7%) | 0.55 | 0.7 | |
| | DPPC | 0.18 | 0.18 | 0.18 |
| | Sodium chloride | 0.02 | 0.02 | 0.02 |
| Analytical | Fine particle fraction, <5.6 um | 57 | 56 | |
| Results | (%), size 00 capsule | | | |
| | Fine particle fraction, <5.6 um | NT | NT | 84 |
| | (%), size 2 capsule | | | |
| | Water content (%) | 5.2 | 3.7 | |
| | gPSD (VMGD 1 bar, mm) | NT | NT | 3.0 |
| | Bulk/Aerated Density (g/cc) | NT | NT | 0.087 |
| | Tap Density (g/cc) | NT | NT | 0.114 |

FPF<5.6 values were observed to be in a suitable range for inhalation, with powder formulation 155136 observed to possess a relatively high FPF<5.6 when assessed via a size 2 inhaler, which should translate into a high efficiency of delivery and resultant efficacy in combination with a reduced potential for adverse taste due to reduced deposition in the oropharyngeal cavity.

### Example 2

### Effect of Variations in DPPC and Zolmitriptan Loads on Spray Dried Zolmitriptan Formulations

This evaluation was performed to understand the effect of zolmitriptan and DPPC loads on the aerosol and solid state properties of spray dried Zolmitriptan formulations. Two DPPC loads (8% and 18%) and two Zolmitriptan (10% and 20%) loads were evaluated. Two carrier combinations were used for the purpose of this evaluation, L-leucine:DPPC:NaCl and SD-30:DPPC:NaCl. A list of the formulations produced is provided in Table 3.

TABLE 3

| | Batch # | Formulation |
|---|---|---|
| SD-30 based | 155137 | 10:70:18:2 Zolmi- |
| formulations | | triptan:SD-30:DPPC:NaCl |
| | 155138 | 10:80:08:2 Zolmi- |
| | | triptan:SD-30:DPPC:NaCl |
| | 155139 | 20:70:08:2 Zolmi- |
| | | triptan:SD-30:DPPC:NaCl |
| | 155140 | 20:60:18:2 Zolmi- |
| | | triptan:SD-30:DPPC:NaCl |
| L-leucine based | 155144 | 10:70:18:2 Zolmi- |
| formulations | | triptan:L-leu:DPPC:NaCl |
| | 155145 | 10:80:08:2 Zolmi- |
| | | triptan:L-leu:DPPC:NaCl |
| | 155146 | 20:70:08:2 Zolmi- |
| | | triptan:L-leu:DPPC:NaCl |
| | 155147 | 20:60:18:2 Zolmi- |
| | | triptan:L-leu:DPPC:NaCl |

Process parameters and analytical results are listed in Table 4 for the SD-30 formulation runs (all runs utilized water as the aqueous solvent and ethanol as the organic solvent). Powders containing SD-30 were observed to possess desired geometric sizes, tap densities and FPF<5.6 values for inhalation. Powders containing less DPPC (8% versus 18%) were seen to possess larger geometric sizes.

TABLE 4

| Lot Number: | | 155137 | 155138 | 155139 | 155140 |
|---|---|---|---|---|---|
| | Total solid concentration | 4 | 4 | 4 | 4 |
| | (g/L) | | | | |
| Process | Inlet Temperature (° C.) | 103 | 103 | 103 | 103 |
| Parameters | Outlet Temperature (° C.) | 60 | 60 | 60 | 60 |
| | Drying Gas Rate (kg/hr) | 100 | 100 | 100 | 100 |
| | Atom. Gas Flow Rate (g/min) | 22 | 22 | 22 | 22 |
| | Aqueous Flow (mL/min) | 8 | 8 | 8 | 8 |
| | Organic flow (mL/min) | 32 | 32 | 32 | 32 |
| | Prod. filter purge flow (scfh) | 15 | 15 | 15 | 15 |
| | Zolmitriptan | 0.1 | 0.1 | 0.2 | 0.2 |
| | Polyglycitol SD-30 | 0.7 | 0.8 | 0.7 | 0.6 |
| | DPPC | 0.18 | 0.08 | 0.08 | 0.18 |
| | Sodium chloride | 0.02 | 0.02 | 0.02 | 0.02 |
| Analytical | Fine particle fraction, <5.6 | 76 | NT | NT | NT |
| Results | um (%), size 00 capsule | | | | |
| | Fine particle fraction, <5.6 | NT | 67 | 66 | 67 |
| | um (%), size 2 capsule | | | | |
| | gPSD (VMGD 1 bar, mm) | 4.0 | 7.4 | 7.5 | 5.4 |
| | Bulk/Aerated Density (g/cc) | 0.049 | 0.036 | 0.042 | 0.041 |
| | Tap Density (g/cc) | 0.077 | 0.060 | 0.073 | 0.062 |

Process parameters and analytical results are listed in Table 4 for the leucine formulation runs (all runs utilized water as the aqueous solvent and ethanol as the organic solvent). For powders containing leucine, FPF<5.6 values were observed to be in a suitable range for inhalation for all 4 formulations, with the powders possessing desired geometric sizes and tap densities. Similar to the case for the SD-30 formulations, powders containing less DPPC were observed to have slightly larger geometric sizes.

TABLE 5

| | | Lot Number | | | |
|---|---|---|---|---|---|
| | | 155144 | 155145 | 155146 | 155147 |
| SD solution | Total solid concentration | 4 | 4 | 4 | 4 |
| concentration | (g/L) | | | | |
| Process | Inlet Temperature (° C.) | 103 | 103 | 103 | 103 |
| Parameters | Outlet Temperature (° C.) | 60 | 60 | 60 | 60 |
| | Drying Gas Rate (kg/hr) | 100 | 100 | 100 | 100 |
| | Atom. Gas Flow Rate (g/min) | 22 | 22 | 22 | 22 |
| | Aqueous Flow (mL/min) | 8 | 8 | 8 | 8 |
| | Organic flow (mL/min) | 32 | 32 | 32 | 32 |
| | Prod. filter purge flow (scfh) | 15 | 15 | 15 | 15 |
| Component | Zolmitriptan | 0.1 | 0.1 | 0.2 | 0.2 |
| Fractions | | | | | |
| | L-leucine | 0.7 | 0.8 | 0.7 | 0.6 |
| | DPPC | 0.18 | 0.08 | 0.08 | 0.18 |
| | Sodium chloride | 0.02 | 0.02 | 0.02 | 0.02 |
| Analytical | Fine particle fraction, <5.6 | 64 | 70 | 71 | 64 |
| results | um (%), size 2 capsule | | | | |
| | gPSD (VMGD 1 bar, mm) | 4.3 | 6.4 | 8.3 | 5.6 |
| | Bulk/Aerated Density (g/cc) | 0.092 | 0.073 | 0.080 | 0.089 |
| | Tap Density (g/cc) | 0.117 | 0.106 | 0.134 | 0.173 |

### Example 3

### Physical Stability (Fine Particle Fraction and Conversion to Crystalline Zolmitriptan Phase) of Selected Formulations from Example 2

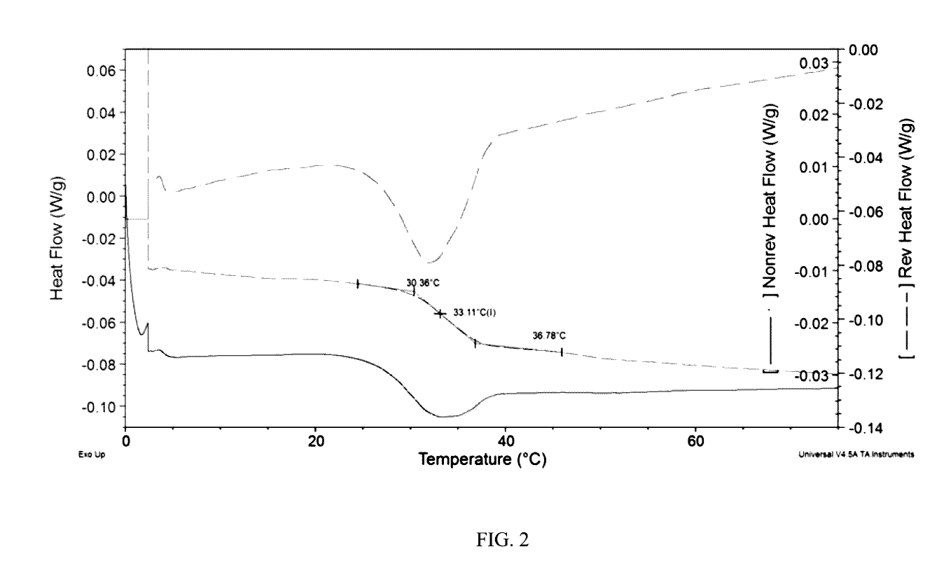

Selected powder lots produced as described in Example 2 were placed on short-term stability at ambient (20° C.) and accelerated (40° C.) temperature storage conditions. For comparative purposes, a 100% spray-dried zolmitriptan powder was prepared and analyzed via XRPD and DSC to allow for a comparison to the formulations from Example 2 that were placed on stability and to facilitate an interpretation of the resultant thermal data. Initially, a lot of amorphous 100% zolmitriptan was produced via a Buchi 290 Spray-Drying System (spray drying parameters: solids concentration=2 g/L, inlet temperature=90° C., outlet temperature=45° C., drying gas flowrate=20 kg/hr, atomization gas flowrate=10 g/min, aqueous flowrate=4 ml/Min, organic phase flowrate=6 ml/min, spray dryer pressure=−50 mbar) and analyzed via modulated DSC (TA Instruments DSC Q2000 Tzero System, MDSC Parameters: hermetically sealed double lid pan configuration, equilibrate at 0° C. for 5 minutes, ramp up to maximum of 160° C. at 2.0° C./min, modulate+/−1.00° C. every 60 seconds) to determine its glass transition temperature. As can be seen in FIG. 2, pure amorphous zolmitriptan was observed to possess a glass transition temperature (Tg) of approximately 33° C., which is relatively low with respect to ambient and accelerated temperature storage conditions and would indicate a poor potential for an amorphous zolmitriptan powder to remain amorphous over extended time periods at these conditions.

Next, for the preparation of 100% zolmitriptan spray-dried powder via the Niro PSD-1 setup shown in FIG. 1, zolmitriptan was dissolved in ethanol and mixed with water (static mixing) immediately prior to atomization. Spray-dried powder was produced utilizing the parameters shown in Table 6.

TABLE 6
Spray-drying parameters utilized to make Batch 290173
(100% zolmitriptan).

| | | Value |
|---|---|---|
| Parameters | Total solid concentration (g/L) | 12 |
| | Inlet Temperature (° C.) | 80 |
| | Outlet Temperature (° C.) | 45 |
| | Drying Gas Rate (kg/hr) | 125 |
| | Atomization Gas Flow Rate | 22 |
| | (g/min) | |
| | Aqueous Flow (mL/min) | 16 |
| | Organic flow (mL/min) | 24 |
| | SD pressure (in WC) | −2 |
| | Product filter purge gas flow | 20 |
| | (scfh) | |
| | Feed temperature (° C.) | 20 |
| Atomization | Air cap | 67147 |
| nozzle | Fluid cap | 2850 |

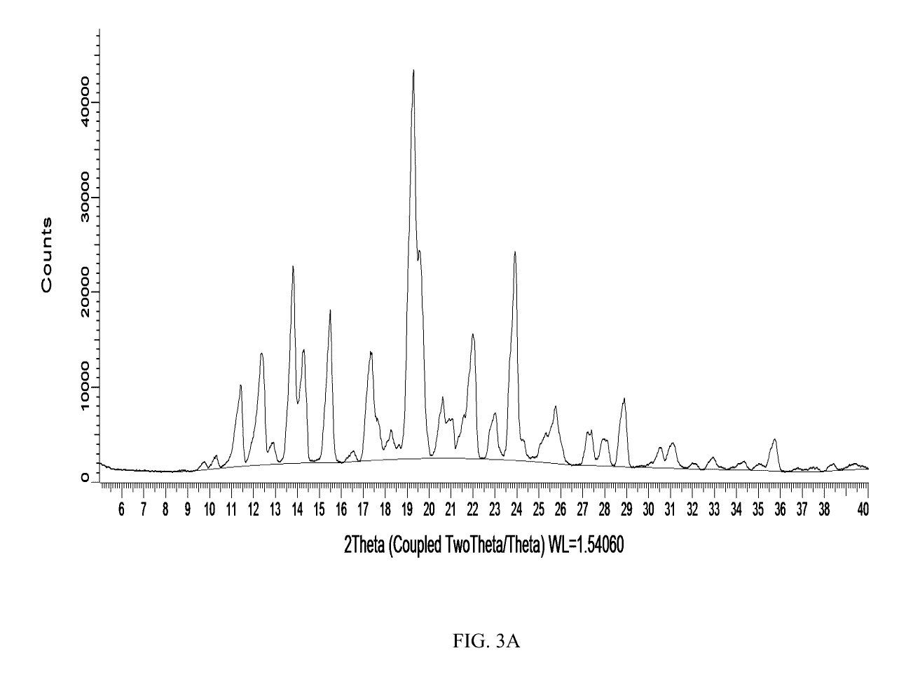

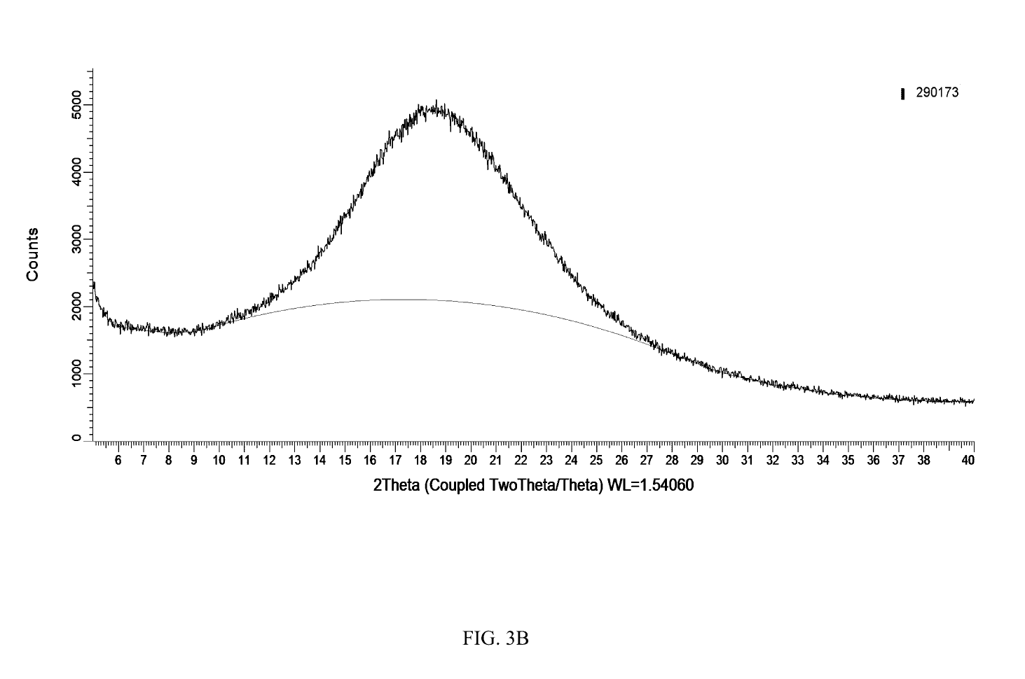

XRPD characterization results for both the resultant 100% zolmitriptan spray-dried material as well as raw crystalline zolmitriptan are shown in FIG. 3, with thermal analysis results summarized in Table 7. Zolmitriptan raw material displayed a melting temperature of approximately 136° C., with a characteristic XRPD pattern represented by several distinct crystalline peaks (FIG. 3A). In contrast, the spray-dried zolmitriptan powder appeared to be completely amorphous at t=0 (FIG. 3B). The spray-dried powder was also relatively cohesive and displayed poor dispersibility and aerodynamic properties that were unsuitable for inhalation, with a FPF<5.6 of approximately 22%. Additionally, after only one week held at 40° C., the powder appears to have been partially converted to a crystalline phase with a melting temperature of 132° C. (Table 7). Such conversion of amorphous powders for inhalation typically results in a drastic reduction in the aerodynamic diameter and dispersibility of the powder due to inter-particle sintering occurring during crystallization (it was observed that the powder was stuck to one side of the capsule after storage for 1 week at 40° C., which prevented ACI analysis for FPF determination).

TABLE 7
Characterization results for batch 290173 (100% spray-dried zolmitriptan).

| | | | | FPF <5.6 | | Low T1 | Tₘ | ΔH |
|---|---|---|---|---|---|---|---|---|
| Batch | Formulation | Condition | | (%) | TGA-120 | (° C.) | (° C.) | (J/g). |
| 290173 | 100% spray- | t = 0 | | 22 | 0.24 | 46.2 | not | NA |
| | dried | | | | | | detected | |
| | zolmitriptan | 40° C. | 1 Wk. | not | 0.25 | 43.9 | 132.2 | 31.9 |
| | | | | tested | | | | |

| | | | | | | |
|---|---|---|---|---|---|---|
| Raw material | t = 0 | NT | NT | not | 136.0 | 135.3 |
| | | | | detected | | |

Pure amorphous zolmitriptan thus has a relatively low Tg with respect to ambient and accelerated storage conditions, and thus would be expected to display very poor physical stability due to conversion to a crystalline phase at these temperatures (due to particle sintering and fusion during conversion to a crystalline phase), which would drastically limit the practicality of use of spray-dried zolmitriptan as a dry powder for inhalation drug product.

Next, powders produced as described in Example 2 above were filled in amber vials and placed under ambient (20° C.) and accelerated temperature (40° C.) stability conditions. Table 8 lists the aerosol property stability results for L-leucine and SD-30 respectively. At t=0, SD-30 based formulations were observed to have a higher FPF<5.6 as compared to the L-leucine based formulations. However, unlike some of the SD-30 based formulations which exhibited a drop in FPF<5.6 over the course of the stability study, all L-leucine based formulations were observed to maintain their FPF<5.6 values.

TABLE 8

| | | | | FPF <5.6 |
|---|---|---|---|---|
| Batch | Formulation | Condition | | (%) |
| 155144 | 10:70:18:2 Zolmitriptan:L- | t = 0 | | 68 |
| | leucine:DPPC:NaCl | 20° C. | 1 Mo. | 61 |
| | | 40° C. | 2 Wk. | 60 |
| | | | 1 Mo. | 67 |
| 155145 | 10:80:8:2 Zolmitriptan:L- | t = 0 | | 70 |
| | leucine:DPPC:NaCl | 20° C. | 1 Mo. | 71 |
| | | 40° C. | 2 Wk. | 72 |
| | | | 1 Mo. | 70 |
| 155146 | 20:70:8:2 Zolmitriptan:L- | t = 0 | | 71 |
| | leucine:DPPC:NaCl | 20° C. | 1 Mo. | 62 |
| | | 40° C. | 2 Wk. | 62 |
| | | | 1 Mo. | 65 |
| 155147 | 20:60:18:2 Zolmitriptan:L- | t = 0 | | 74 |
| | leucine:DPPC:NaCl | 20° C. | 1 Mo. | 51 |
| | | 40° C. | 2 Wk. | 57 |
| | | | 1 Mo. | 63 |
| 155137 | 10:70:18:2 Zolmitriptan:SD- | t = 0 | | 76 |
| | 30:DPPC:NaCl | 20° C. | 2 Wk. | 72 |
| | | | 1 Mo. | 68 |
| | | 40° C. | 2 Wk. | 73 |
| | | | 1 Mo. | 76 |
| 155138 | 10:80:8:2 Zolmitriptan:SD- | t = 0 | | 67 |
| | 30:DPPC:NaCl | 20° C. | 2 Wk. | 70 |
| | | | 1 Mo. | 51 |
| | | 40° C. | 2 Wk. | 64 |
| | | | 1 Mo. | 50 |
| 155139 | 20:70:8:2 Zolmitriptan:SD- | t = 0 | | 66 |
| | 30:DPPC:NaCl | 20° C. | 2 Wk. | 68 |
| | | | 1 Mo. | 33 |
| | | 40° C. | 2 Wk. | 63 |
| | | | 1 Mo. | 31 |
| 155140 | 20:60:18:2 Zolmitriptan:SD- | t = 0 | | 67 |
| | 30:DPPC:NaCl | 20° C. | 2 Wk. | 75 |
| | | | 1 Mo. | 65 |
| | | 40° C. | 2 Wk. | 67 |
| | | | 1 Mo. | 49 |

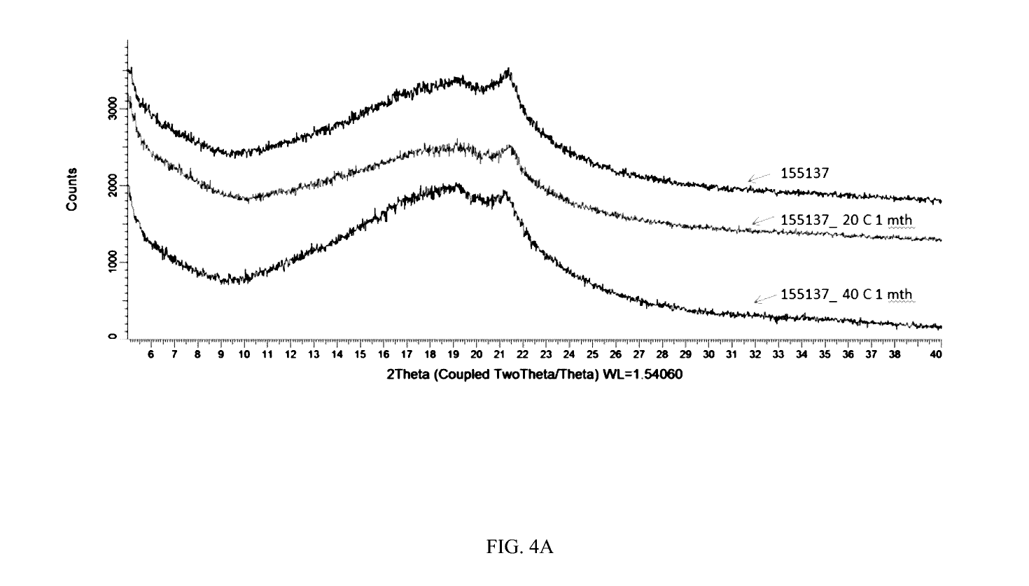

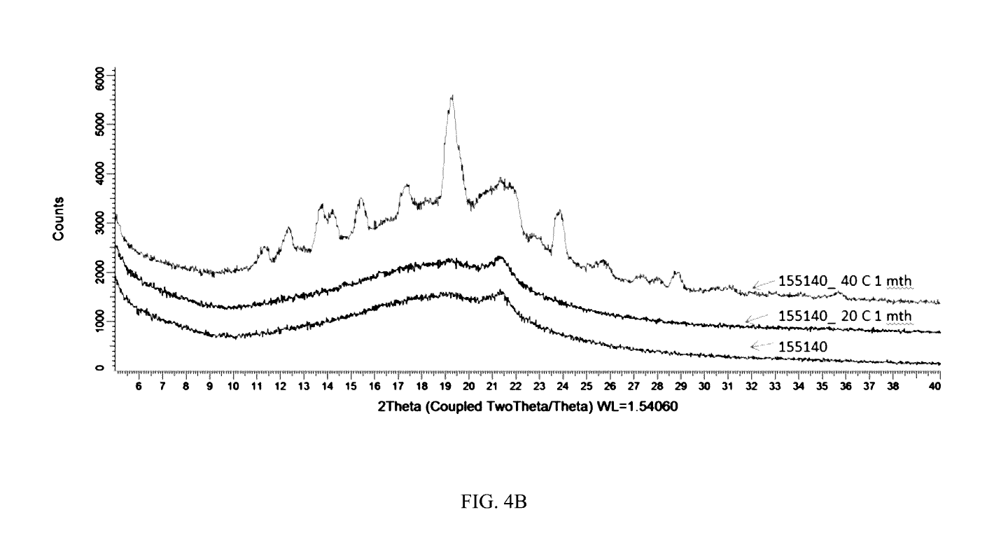

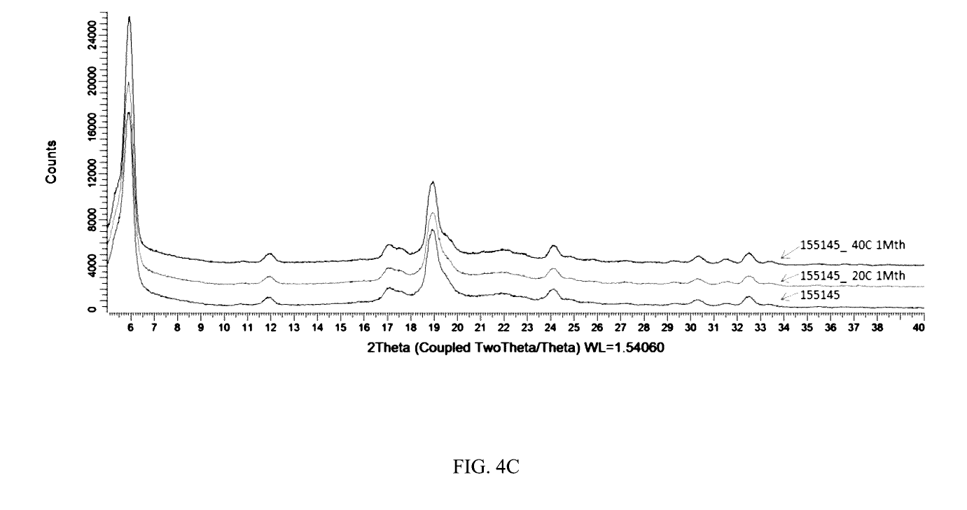

Table 9 lists the solid state characterization results for L-leucine and SD-30 respectively, with representative XRPD profiles for 155137, 155140 and 155145 are shown in FIG. 4. The t=0 thermal and XRPD characterization data from Table 9 and FIG. 4 indicates that zolmitriptan is present as part of an amorphous solid-dispersion matrix in SD-30 formulations with partially ordered DPPC and NaCl, as indicated by the A-T1 XRPD pattern, which is represented by no sign of characteristic peaks for zolmitriptan with only 2 broad peaks characteristic of a lamellar DPPC phase being present, and as is further indicated by the presence of low temperature thermal events in the range of the Tg observed for 100% amorphous zolmitriptan. In contrast, zolmitriptan is present in a separate amorphous state in combination with a predominantly crystalline leucine matrix in formulations containing leucine with DPPC and NaCl, as indicated by the PC-T2 XRPD pattern, which is represented by several peaks attributable to crystalline leucine being present with no peaks attributable to crystalline zolmitriptan, and as further indicated by the presence of low temperature thermal events in the range of the observed Tg for 100% amorphous zolmitriptan. As can be seen in FIG. 4B, the drop in FPF<5.6 seen on stability at 40° C. for SD-30 formulation 155140 corresponds with the observance of a newly-formed zolmitriptan crystalline phase, which likely acts to decrease FPF<5.6 and dispersibility via the formation of interparticulate bridges due to sintering during the crystallization of zolmitriptan. In contrast, no changes are seen in the XRPD profile over time at 40° C. for the leucine-based formulations. It thus appears that, surprisingly, leucine is more effective in stabilizing amorphous zolmitriptan in the solid-state than SD-30 despite these differences and despite the known tendency of solid dispersion-forming agents like SD-30 to stabilize amorphous drug phases. It is also very surprising that zolmitriptan appears to be present in an amorphous phase with a low Tg (range of 30 to 40° C.) for both the SD-30-based and leucine-based formulations, yet these powders remain stable at over extended storage times at 40° C. for several of the SD-30-based formulations and all of the leucine-based formulations.

TABLE 9

| | | | | TGA-120 | Low T1 | Low T2 | | |
|---|---|---|---|---|---|---|---|---|
| Batch | Formulation | Condition | | (%) | (° C.) | (° C.) | Recryst. | Melt |
| 155144 | 10:70:18:2 | t = 0 | | 0.5 | 33 | 58.8, 69 | n.d. | n.d. |
| | Zolmitriptan:L- | 20° C. | 1 Mo. | 0.72 | 39, 51 | 58.9, 68.9 | n.d. | 140 (broad) |
| | leu:DPPC:NaCl | 40° C. | 2 Wk. | 0 (chg) | 50, 58 | 68.3 | n.d. | 135 (broad) |
| | | | 1 Mo. | 0.23 | 39, 51 | 58.7, 68.9 | n.d. | 140 (broad) |
| 155145 | 10:80:8:2 | t = 0 | | 0.22 | 48.8 | n.d. | n.d. | n.d. |
| | Zolmitriptan:L- | 20° C. | 1 Mo. | 0.22 | 38, 50 | 59.2, 68.8 | n.d. | 140 (broad) |
| | leu:DPPC:NaCl | 40° C. | 2 Wk. | 0.17 | 42, 49 | 59.4, 69.2 | n.d. | 140 (broad) |
| | | | 1 Mo. | 0 (chg) | 42, 52 | 69.2 | n.d. | 150 (broad) |
| 155146 | 20:70:8:2 | t = 0 | | 0.13 | 32.5 | 53.8 | n.d. | n.d. |
| | Zolmitriptan:L- | 20° C. | 1 Mo. | 0.42 | 51.1 | 58.7, 68.3 | n.d. | 130 (broad) |
| | leu:DPPC:NaCl | 40° C. | 2 Wk. | 0.25 | 49, 54 | 58.6, 68.1 | n.d. | 120 (broad) |
| | | | 1 Mo. | 0 (chg) | 39, 52 | 58.9, 68.9 | n.d. | 150 (broad) |
| 155147 | 20:60:18:2 | t = 0 | | 0.11 | 32.9 | 56.3 | n.d. | n.d. |
| | Zolmitriptan:L- | 20° C. | 1 Mo. | 0.32 | 34, 51 | 59.1, 69.3 | n.d. | n.d. |
| | leu:DPPC:NaCl | 40° C. | 2 Wk. | 0.47 | 50.2 | 57, 67.8 | n.d. | 120 (broad) |
| | | | 1 Mo. | 0.56 | 33, 51 | 58.5, 68.7 | n.d. | 130 (broad) |
| 155136 | 10:70:18:2 | t = 0 | | 3.62 | 39.2, 45.6 | 60.6 | n.d. | n.d. |
| | Zolmitriptan:SD- | 20° C. | 2 Wk. | 3.84 | 39.5, 45.3 | 61 | n.d. | n.d. |
| | 30:DPPC:NaCl | | 1 Mo. | 2.68 | 37.5 | 62.7 | n.d. | n.d. |
| | | 40° C. | 2 Wk. | 3.8 | 39, 47 | 62.8 | n.d. | n.d. |
| | | | 1 Mo. | 2.97 | 42, 54 | 81.9 | n.d. | n.d. |
| 155137 | 10:70:18:2 | t = 0 | | 3.11 | 39.7 | 39.7 | n.d. | n.d. |
| | Zolmitriptan:SD- | 20° C. | 2 Wk. | 1.94 | 40.3 | 40.3 | n.d. | n.d. |
| | 30:DPPC:NaCl | | 1 Mo. | 2.48 | 38 | 38 | n.d. | n.d. |
| | | 40° C. | 2 Wk. | 2.44 | 40.4 | 40.4 | n.d. | n.d. |
| | | | 1 Mo. | 2.61 | 37.5 | 37.5 | n.d. | n.d. |
| 155138 | 10:80:8:2 | t = 0 | | 3.89 | 40 | 40 | n.d. | n.d. |
| | Zolmitriptan:SD- | 20° C. | 2 Wk. | 3.35 | 40.9 | 40.9 | n.d. | n.d. |
| | 30:DPPC:NaCl | | 1 Mo. | 1.88 | 37.3 | 37.3 | n.d. | n.d. |
| | | 40° C. | 2 Wk. | 3.19 | 41.2 | 41.2 | n.d. | n.d. |
| | | | 1 Mo. | 3.25 | 38.4 | 38.4 | n.d. | n.d. |
| 155139 | 20:70:8:2 | t = 0 | | 5.64 | 39.5 | 39.5 | n.d. | n.d. |
| | Zolmitriptan:SD- | 20° C. | 2 Wk. | 4.45 | 40.1 | 40.1 | n.d. | n.d. |
| | 30:DPPC:NaCl | | 1 Mo. | 3.05 | 41.6 | 41.6 | n.d. | 122.4 |
| | | 40° C. | 2 Wk. | 3.77 | 40.6 | 40.6 | n.d. | n.d. |
| | | | 1 Mo. | 4.27 | 40.3 | 40.3 | n.d. | 117 |
| 155140 | 20:60:18:2 | t = 0 | | 3.45 | 39.7 | n.d. | n.d. | n.d. |
| | Zolmitriptan:SD- | 20° C. | 2 Wk. | 3.47 | 40.3 | n.d. | n.d. | n.d. |
| | 30:DPPC:NaCl | | 1 Mo. | 1.75 | 37.7 | 50.8 | n.d. | n.d. |
| | | 40° C. | 2 Wk. | 3.53 | 40.7 | n.d. | n.d. | n.d. |
| | | | 1 Mo. | 3.53 | 41 | 70 (broad) | n.d. | 122.1 |

These results thus confirm the poor physical stability of amorphous 100% zolmitriptan powders due to their relatively low T_{g}s, in contrast to the stabilized zolmitriptan formulations disclosed herein.

### Example 4

### Evaluation of Different Maltodextrins with Respect to Physical Stability

This evaluation was performed to compare the effectiveness of three different types of polyglycitols/maltodextrins in 15% Zolmitriptan spray dried formulations with respect to physical stability at ambient and accelerated storage conditions (as represented by FPF<5.6), these being Polyglycitol SD-30, Maltrin M-200, and Maltrin M-250. The process parameters described in Example 2 were used for this evaluation. Samples produced for this evaluation were filled in capsules, blister packaged and stored for a period of up to 1 month at 20 and 40° C. temperatures. Table 10 details the aerosol evaluation results, and Table 11 details the solid state analytical testing results (all powders tested displayed a consistent A-T1 XRPD pattern and showed no indication of a zolmitriptan melting transition via DSC). Under the formulation ratios and processing conditions utilized, all three formulations were stable with respect to FPF<5.6 after up to 1 month storage at 40° C.

TABLE 10

| | | | | FPF <5.6 |
|---|---|---|---|---|
| Batch | Formulation | Condition | | (%) |
| 189018 | 15:65:18:2 Zolmitriptan:M- | t = 0 | | 73 |
| | 250:DPPC:NaCl | 20° C. | 2 Wk. | 62 |
| | | | 1 Mo. | 72 |
| | | 40° C. | 2 Wk. | 76 |
| | | | 1 Mo. | 72 |
| 189019 | 15:65:18:2 Zolmitriptan:M- | t = 0 | | 71 |
| | 200:DPPC:NaCl | 20° C. | 2 Wk. | 63 |
| | | | 1 Mo. | 66 |
| | | 40° C. | 2 Wk. | 79 |
| | | | 1 Mo. | 67 |
| 189020 | 15:65:18:2 Zolmitriptan:SD- | t = 0 | | 65 |
| | 30:DPPC:NaCl | 20° C. | 2 Wk. | 63 |
| | | | 1 Mo. | 72 |
| | | 40° C. | 2 Wk. | 66 |
| | | | 1 Mo. | 68 |

TABLE 11

| | | | | | | | | Recryst. |
|---|---|---|---|---|---|---|---|---|
| | | | | TGA-120 | Low T 1 | Low T 2 | Recryst. | ΔH |
| Batch | Formulation | Condition | | (%) | (° C.) | (° C.) | (° C.) | (J/g) |
| 189018 | 15:65:18:2 | t = 0 | | 3.21 | 39.1 | 42.6, 45.3 | 61.6 (broad) | 2.23 |
| | Zolmitrip- | 20° C. | 1 Mo. | 2.93 | 42.8 | 45.1, 50.6 | 69.8 (broad) | 2.67 |
| | tan:M250:DPPC:NaCl | 40° C. | 1 Mo. | 2.3 | 43.0 | 45.9, 53.5 | 77.3 (broad) | 2.2 |
| 189019 | 15:65:18:2 | t = 0 | | 2.53 | 38.9 | 42 | 64.7 (broad) | 2.07 |
| | Zolmitrip- | 20° C. | 1 Mo. | 1.84 | 45.6 | 48.1 | 67.2 (broad) | 2.15 |
| | tan:M200:DPPC:NaCl | 40° C. | 1 Mo. | 2.04 | 41.1 | 45.6, 57.3 | 71.6 (broad) | 0.311 |
| 189020 | 15:65:18:2 | t = 0 | | 2.9 | 37.8 | 40.8, 43.3 | 63.5 (broad) | 1.5 |
| | Zolmitrip- | 20° C. | 1 Mo. | 2.41 | 39.9 | 42.8, 48.8 | 64.8 (broad) | 2.27 |
| | tan:SD30:DPPC:NaCl | 40° C. | 1 Mo. | 2.14 | 37.3 | 39.5, 52.9 | 65.3 (broad) | 0.34 |

### Example 5

### Effect of Varied DPPC:NaCl Ratio

This evaluation was performed to screen different DPPC:NaCl ratios in a 15% Zolmitriptan formulation with SD-30. The process parameters described in Example 2 were used for producing the powders that were to be evaluated. Five different ratios were evaluated and the powders produced during this evaluation were filled in capsules, blister packaged and stored for a period of up to 1 month at 20 and 40° C. temperatures. Table 12 lists the aerosol data and Table 13 lists the solid state data obtained after evaluating stability samples. Increasing the amount of sodium chloride relative to DPPC resulted in an increase in particle size and a decrease in FPF; stability to physical conversion, as indicated by the lack of a significant drop in FPF<5.6, was good for all formulations tested.

TABLE 12

| | | | | | FPF <5.6 |
|---|---|---|---|---|---|
| Batch | Formulation | Condition | | VMGD (um) | (%) |
| 189020 | 15:65:18:2 | t = 0 | | 5.4 | 65 |
| | Zolmitriptan:SD- | 20° C. | 2 Wk. | NT | 63 |
| | 30:DPPC:NaCl | | 1 Mo. | NT | 72 |
| | | 40° C. | 2 Wk. | NT | 66 |
| | | | 1 Mo. | NT | 68 |
| 189021 | 15:65:15:5 | t = 0 | | 5.7 | 56 |
| | Zolmitriptan:SD- | 20° C. | 2 Wk. | NT | 59 |
| | 30:DPPC:NaCl | | 1 Mo. | NT | 66 |
| | | 40° C. | 2 Wk. | NT | 63 |
| | | | 1 Mo. | NT | 66 |
| 189023 | 15:65:12:8 | t = 0 | | 6.7 | 58 |
| | Zolmitriptan:SD- | 20° C. | 2 Wk. | NT | 56 |
| | 30:DPPC:NaCl | | 1 Mo. | NT | 65 |
| | | 40° C. | 2 Wk. | NT | 61 |
| | | | 1 Mo. | NT | 66 |
| 189024 | 15:65:8:12 | t = 0 | | 10.2 | 55 |
| | Zolmitriptan:SD- | 20° C. | 2 Wk. | NT | 54 |
| | 30:DPPC:NaCl | | 1 Mo. | NT | 65 |
| | | 40° C. | 2 Wk. | NT | 66 |
| | | | 1 Mo. | NT | 68 |
| 189026 | 15:65:0:20 | t = 0 | | 12.4 | 46 |
| | Zolmitriptan:SD- | 20° C. | 2 Wk. | NT | 55 |
| | 30:DPPC:NaCl | | 1 Mo. | NT | 49 |
| | | 40° C. | 2 Wk. | NT | 48 |
| | | | 1 Mo. | NT | 50 |

TABLE 13

| | | | | | | | | Recryst. | |
|---|---|---|---|---|---|---|---|---|---|
| | | | | TGA-120 | Low T 1 | Low T 2 | Recryst. | ΔH | Melt |
| Batch | Formulation | Condition | XRPD | (%) | (° C.) | (° C.) | (° C.) | (J/g) | (° C.) |
| 189020 | 15:65:18:2 | t = 0 | A-T1 | 2.9 | 37.8 | 40.8, 43.3 | 63.5 (broad) | 1.5 | n.d. |
| | Zolmitriptan:SD- | 20° C., 1 Mo | A-T1 | 2.41 | 39.9 | 42.8, 48.8 | 64.8 (broad) | 2.27 | n.d. |
| | 30:DPPC:NaCl | 40° C., 1 Mo. | A-T1 | 2.14 | 37.3 | 39.5, 52.9 | 65.3 (broad) | 0.34 | n.d. |
| 189021 | 15:65:15:5 | t = 0 | A-T1 | 3.01 | 38.5 | 41.9 | 62.3 (broad) | 1.08 | 162.4 |
| | Zolmitriptan:SD- | 20° C., 1 Mo | A-T1 | 2.17 | 39.5 | 41.7, 45.6 | 61.4 (broad) | 1.74 | n.d. |
| | 30:DPPC:NaCl | 40° C., 1 Mo. | A-T1 | 2.15 | 41.4 | 44.3, 56.5, 82.6 | n.d. | n.d. | n.d. |
| 189023 | 15:65:12:8 | t = 0 | A-T2 | 3.26 | 47.9 | 44.5, 45.8 | 62.5 (broad) | 1.66 | 167 |
| | Zolmitriptan:SD- | 20° C., 1 Mo | A-T2 | 2.87 | 42.4 | 48 | 61.3 (broad) | 3.09 | 162.2 |
| | 30:DPPC:NaCl | 40° C., 1 Mo. | A-T2 | 2.34 | 40.7 | 42.7, 54.7, 70.7 | n.d. | n.d. | n.d. |
| 189024 | 15:65:8:12 | t = 0 | A-T2 | 3.38 | 38.2 | 40.5 | 51.3 (broad) | 3 | n.d. |
| | Zolmitriptan:SD- | 20° C., 1 Mo | A-T2 | 1.76 | 45.4 | 48.2 | 62.3 (broad) | 4.04 | n.d. |
| | 30:DPPC:NaCl | 40° C., 1 Mo. | A-T2 | 2.02 | 40.5, 50.7 | 42.8, 54.6, 78.8 | n.d. | n.d. | 166.8 |
| 189026 | 15:65:0:20 | t = 0 | A-T2 | 3.18 | 40.3 | 52.6 | 59.0 (broad) | 0.823 | 105.1 |
| | Zolmitriptan:SD- | 20° C., 1 Mo | A-T2 | 2.62 | 41.8 | 52.3 | 62.2 (broad) | 4.44 | 164? |
| | 30:DPPC:NaCl | 40° C., 1 Mo. | A-T2 | 1.97 | 41 | 55.7 | 64.5 (broad) | 1.19 | n.d. |

### Example 6

### Effect of Substituting NaCl with NaCitrate

This evaluation was performed to observe the effect of substituting sodium chloride with sodium citrate in a 15% Zolmitriptan formulation. Two loads of both salts were tested out, these being 5% and 20%. Process parameters listed in Example 2 were used for producing the powders that were to be evaluated. Powders produced during this evaluation were filled in capsules, blister packaged and stored for a period of up to 1 month at 20 and 40° C. temperatures. Table 14 summarizes the aerosol stability data and Table 15 summarizes the solid state stability data for this evaluation. Sodium citrate produced similar results to those seen for sodium chloride. Powders having no DPPC have a much higher gPSD as compared to the ones with DPPC. FPFs of powders containing DPPC were much higher than those without DPPC, and the FPFs of all powders were stable over the course of this stability evaluation.

TABLE 14

| | | | | VMGD | FPF <5.6 |
|---|---|---|---|---|---|
| Batch | Formulation | Condition | | (um) | (%) |
| 189021 | 15:65:15:5 | t = 0 | | 5.7 | 56 |
| | Zolmitriptan:SD- | 20° C. | 2 Wk. | NT | 59 |
| | 30:DPPC:NaCl | | 1 Mo. | NT | 66 |
| | | 40° C. | 2 Wk. | NT | 63 |
| | | | 1 Mo. | NT | 66 |
| 189022 | 15:65:15:5 | t = 0 | | 5.9 | 64 |
| | Zolmitriptan:SD- | 20° C. | 2 Wk. | NT | 63 |
| | 30:DPPC:NaCitrate | | 1 Mo. | NT | 66 |
| | | 40° C. | 2 Wk. | NT | 70 |
| | | | 1 Mo. | NT | 65 |
| 189026 | 15:65:20 | t = 0 | | 12.4 | 46 |
| | Zolmitriptan:SD- | 20° C. | 2 Wk. | NT | 55 |
| | 30:NaCl | | 1 Mo. | NT | 49 |
| | | 40° C. | 2 Wk. | NT | 48 |
| | | | 1 Mo. | NT | 50 |
| 189027 | 15:65:20 | t = 0 | | 15.3 | 44 |
| | Zolmitriptan:SD- | 20° C. | 2 Wk. | NT | 62 |
| | 30:NaCitrate | | 1 Mo. | NT | 44 |
| | | 40° C. | 2 Wk. | NT | 41 |
| | | | 1 Mo. | NT | 44 |

TABLE 15

| | | | | | | | | Recryst. | |
|---|---|---|---|---|---|---|---|---|---|
| | | | | TGA-120 | Low T 1 | Low T 2 | Recryst. | ΔH | Melt |
| Batch | Formulation | Condition | XRPD | (%) | (° C.) | (° C.) | (° C.) | (J/g) | (° C.) |
| 189021 | 15:65:15:5 | t = 0 | A-T1 | 3.01 | 38.5 | 41.9 | 62.3 (broad) | 1.08 | 162.4 |
| | Zolmitriptan:SD- | 20° C., 1 Mo. | A-T1 | 2.17 | 39.5 | 41.7, 45.6 | 61.4 (broad) | 1.74 | n.d. |
| | 30:DPPC:NaCl | 40° C., 1 Mo. | A-T1 | 2.15 | 41.4 | 44.3, 56.5, 82.6 | n.d. | n.d. | n.d. |
| 189022 | 15:65:15:5 | t = 0 | A-T1 | 3.12 | 39.3 | 41.7 | 58.9 (broad) | 2.16 | 159.1 |
| | Zolmitriptan:SD- | 20° C., 1 Mo. | A-T1 | 3.01 | 40.4 | 47 | 61.7 (broad), 81.3 | 1.48 | 169.3 |
| | 30:DPPC:NaCitrate | 40° C., 1 Mo. | A-T1 | 2.39 | 39.1 | 41.0, 53.0, 71.8 | n.d. | n.d. | 171.1 |
| 189026 | 15:65:20 | t = 0 | A-T2 | 3.18 | 40.3 | 52.6 | 59.0 (broad) | 0.823 | 105.1 |
| | Zolmitriptan:SD- | 20° C., 1 Mo. | A-T2 | 2.62 | 41.8 | 52.3 | 62.2 (broad) | 4.44 | 164 |
| | 30:NaCl | 40° C., 1 Mo. | A-T2 | 1.97 | 41 | 55.7 | 64.5 (broad) | 1.19 | n.d. |
| 189027 | 15:65:20 | t = 0 | A-T2 | 2.91 | 40.3 | 52.4 | n.d. | n.d. | 157 |
| | Zolmitriptan:SD- | 20° C., 1 Mo. | A-T2 | 2.51 | 41.3 | 52.4 | n.d. | n.d. | n.d. |
| | 30:NaCitrate | 40° C., 1 Mo. | A-T2 | 2.33 | 41.3 | broad | n.d. | n.d. | n.d. |

### Example 7

### Effect of Substituting DPPC with DSPC

This evaluation was performed to observe the effect of substituting DSPC for DPPC in a 15% Zolmitriptan formulation with SD-30 and NaCl. Process parameters listed in Example 2 were used for producing the powders that were to be evaluated. Powders produced during this evaluation were filled in capsules, blister packaged and stored for a period of up to 1 month at 20 and 40° C. temperatures. Table 16 details FPF<5.6 and gPSD measurements, and Table 17 details measurements of the solid state properties over the course of this stability study. The gPSD of the DSPC formulation was much higher than the DPPC formulation. The FPF<5.6 of the DPPC formulations was higher than the DSPC formulations. However, both formulations have a relatively stable FPF<5.6 value over the course of this evaluation.

TABLE 16

| | | | | | FPF <5.6 |
|---|---|---|---|---|---|
| Batch | Formulation | Condition | | VMGD (um) | (%) |
| 189020 | 15:65:18:2 | t = 0 | | 5.4 | 65 |
| | Zolmitriptan:SD- | 20° C. | 2 Wk. | NT | 63 |
| | 30:DPPC:NaCl | | 1 Mo. | NT | 72 |
| | | 40° C. | 2 Wk. | NT | 66 |
| | | | 1 Mo. | NT | 68 |
| 189025 | 15:65:18:2 | t = 0 | | 12 | 48 |
| | Zolmitriptan:SD- | 20° C. | 2 Wk. | NT | 52 |
| | 30:DSPC:NaCl | | 1 Mo. | NT | 51 |
| | | 40° C. | 2 Wk. | NT | 53 |
| | | | 1 Mo. | NT | 59 |

TABLE 17

| | | | | | | | | | Recryst. | |
|---|---|---|---|---|---|---|---|---|---|---|
| | | | | | TGA-120 | Low T 1 | Low T 2 | Recryst. | ΔH | Melt |
| Batch | Formulation | Condition | | XRPD | (%) | (° C.) | (° C.) | (° C.) | (J/g) | (° C.) |
| 189020 | 15:65:18:2 | t = 0 | | A-T1 | 2.9 | 37.8 | 40.8, 43.3 | 63.5 (broad) | 1.5 | n.d. |
| | Zolmitriptan:SD- | 20° C. | 1 Mo. | A-T1 | 2.41 | 39.9 | 42.8, 48.8 | 64.8 (broad) | 2.27 | n.d. |
| | 30:DPPC:NaCl | 40° C. | 1 Mo. | A-T1 | 2.14 | 37.3 | 39.5, 52.9 | 65.3 (broad) | 0.34 | n.d. |
| 189025 | 15:65:18:2 | t = 0 | | A-T2 | 2.36 | 39.4 | 51.6 | 64.3 (broad) | 2.42 | n.d. |
| | Zolmitriptan:SD- | 20° C. | 1 Mo. | A-T2 | 2.23 | 40.1 | 51.9 | 63.6 (broad) | 5.56 | 166.6 |
| | 30:DSPC:NaCl | 40° C. | 1 Mo. | A-T2 | 2.03 | 41.2 | 55.8 | 65.5, 81.6 | 1.49, 1.66 | n.d. |
| | | | | | | | | (both broad) | | |

### Example 8

### Effect of L-Leucine Addition to SD-30 Based Formulation

This evaluation was performed to observe the effect of addition of L-leucine on the stability of 15% Zolmitriptan formulations with SD-30, DPPC and NaCl. Process parameters listed in Example 2 were used for spray drying these formulations. These powders were filled in capsules, blister packaged and stored for a period of up to 1 month at 20 and 40° C. temperatures. Table 18 details FPF<5.6 and gPSD measurements, and Table 19 details measurements of the solid state properties over the course of this stability study. All powders have a relatively stable FPF<5.6 over the course of this stability evaluation. The FPF<5.6 for the 5% l-leucine formulation (Batch 189028) was lower and the gPSD was higher as compared to the other formulations.

TABLE 18

| | | | | VMGD | FPF <5.6 |
|---|---|---|---|---|---|
| Batch | Formulation | Condition | | (um) | (%) |
| 189020 | 15:65:18:2 | t = 0 | | 5.4 | 65 |
| | Zolmitriptan:SD- | 20° C. | 2 Wk. | NT | 63 |
| | 30:DPPC:NaCl | | 1 Mo. | NT | 72 |
| | | 40° C. | 2 Wk. | NT | 66 |
| | | | 1 Mo. | NT | 68 |
| 189028 | 15:60:5:18:2 | t = 0 | | 15.5 | 41 |
| | Zolmitriptan:SD-30:L- | 20° C. | 2 Wk. | NT | 43 |
| | leu:DPPC:NaCl | | 1 Mo. | NT | 47 |
| | | 40° C. | 2 Wk. | NT | 44 |
| | | | 1 Mo. | NT | 44 |
| 189029 | 15:55:10:18:2 | t = 0 | | 6 | 60 |
| | Zolmitriptan:SD- | 20° C. | 2 Wk. | NT | 62 |
| | 30:L-leu:DPPC:NaCl | | 1 Mo. | NT | 61 |
| | | 40° C. | 2 Wk. | NT | 61 |
| | | | 1 Mo. | NT | 58 |
| 189030 | 15:65:18:2 | t = 0 | | 5.7 | 72 |
| | Zolmitriptan:L- | 20° C. | 2 Wk. | NT | 68 |
| | leu:DPPC:NaCl | | 1 Mo. | NT | 71 |
| | | 40° C. | 2 Wk. | NT | 67 |
| | | | 1 Mo. | NT | 64 |

TABLE 19

| | | | | | | | | Recryst. | |
|---|---|---|---|---|---|---|---|---|---|
| | | | | TGA-120 | Low T 1 | Low T 2 | Recryst. | ΔH | Melt |
| Batch | Formulation | Condition | | (%) | (° C.) | (° C.) | (° C.) | (J/g) | (° C.) |
| 189020 | 15:65:18:2 | t = 0 | | 2.9 | 37.8 | 40.8, 43.3 | 63.5 (broad) | 1.5 | n.d. |
| | Zolmitriptan:SD- | 20° C. | 1 Mo. | 2.41 | 39.9 | 42.8, 48.8 | 64.8 (broad) | 2.27 | n.d. |
| | 30:DPPC:NaCl | 40° C. | 1 Mo. | 2.14 | 37.3 | 39.5, 52.9 | 65.3 (broad) | 0.34 | n.d. |
| 189028 | 15:60:5:18:2 | t = 0 | | 3.93 | 39.7 | 52.1 | unclear | unclear | n.d. |
| | Zolmitriptan:SD- | 20° C. | 1 Mo. | 2.6 | 41.4 | 52.7 | unclear | unclear | n.d. |
| | 30:L-leu:DPPC:NaCl | 40° C. | 1 Mo. | 2.45 | 39.6 | 55.9, 79.6 | unclear | unclear | n.d. |
| 189029 | 15:55:10:18:2 | t = 0 | | 2.82 | 38.5 | 41.3, 43.1 | 56.1 (broad) | 4.85 | 150 |
| | Zolmitriptan:SD- | 20° C. | 1 Mo. | 2.17 | ~42 | 49.4 | unclear | unclear | n.d. |
| | 30:L-leu:DPPC:NaCl | 40° C. | 1 Mo. | 1.99 | 41.7 | 43.5 | 58.7 (broad) | 1.85 | n.d. |
| 189030 | 15:65:18:2 | t = 0 | | 1.47 | 37.4 | 40.8, 55.8 | 68.5 (broad) | 3.45 | 155 |
| | Zolmitriptan:L- | 20° C. | 1 Mo. | 1.24 | 42.5 | 47.6 | 68.5 (broad) | 5.02 | 168 |
| | leu:DPPC:NaCl | 40° C. | 1 Mo. | 0.78 | 40.1 | 43.4, 57.3 | 68.3 (broad) | 3.94 | 170 |

### Example 9

### Effect of Varied SD-30:DPPC Ratio at a Constant Zolmitriptan Load

This evaluation was performed to understand the effect of varied SD-30:DPPC ratios on the aerosol and solid state properties of spray dried Zolmitriptan formulations. Powders produced using DPPC loads of 18%, 38%, 58%, and 78% were filled in capsules, blister packaged and stored for a period of up to 1 month at 20 and 40° C. temperatures. Process parameters used for the production of these powders were similar to those shown in Example 2. Table 20 details FPF<5.6 and gPSD measurements, and Table 21 details measurements of the solid state properties over the course of this stability study. The powder having an 18% load of DPPC was observed to have a higher gPSD as compared to the other powders. The FPF<5.6 of all powders tested remained relatively stable over all storage conditions.

TABLE 20

| | | | | VMGD | FPF <5.6 |
|---|---|---|---|---|---|
| Batch | Formulation | Condition | | (um) | (%) |
| 189082 | 15:65:18:2 | t = 0 | | 6.4 | 73 |
| | ZolmitriptanFB:SD- | 40° C. | 2 Wk. | NT | 73 |
| | 30:DPPC:NaCl | | 1 Mo. | NT | 74 |
| 189083 | 15:45:38:2 | t = 0 | | 3.7 | 75 |
| | ZolmitriptanFB:SD- | 40° C. | 2 Wk. | NT | 65 |
| | 30:DPPC:NaCl | | 1 Mo. | NT | 74 |
| 189084 | 15:25:58:2 | t = 0 | | 3.1 | 73 |
| | ZolmitriptanFB:SD- | 40° C. | 2 Wk. | NT | 66 |
| | 30:DPPC:NaCl | | 1 Mo. | NT | 77 |
| 189085 | 15:5:78:2 | t = 0 | | 4.3 | 64 |
| | ZolmitriptanFB:SD- | 40° C. | 2 Wk. | NT | 52 |
| | 30:DPPC:NaCl | | 1 Mo. | NT | 68 |

TABLE 21

| | | | | | | | | | Recryst. | |
|---|---|---|---|---|---|---|---|---|---|---|
| | | | | | TGA-120 | Low T 1 | Low T 2 | Recryst. | ΔH | Melt |
| Batch # | Formulation | Condition | | XRPD | (%) | (° C.) | (° C.) | (° C.) | (J/g) | (° C.) |
| 189082 | 15:65:18:2 | t = 0 | | A-T2 | 3.64 | 36.9 | 39.4 | 52.9, 65.5 | n.c., 1.45 | n.d. |
| | ZolmitriptanFB:SD- | 40° C. | 1 Mo. | A-T2 | 3.48 | 37.1 | 39 | 54.8, 74.8 | n.c., n.c. | n.d. |
| | 30:DPPC:NaCl | | | | | | | | | |
| 189083 | 15:45:38:2 | t = 0 | | A-T2 | 2.76 | 36.3 | 39.0, 51.7 | 66.9 | n.c | 157 |
| | ZolmitriptanFB:SD- | 40° C. | 1 Mo. | A-T2 | 2.61 | 36.7 | 39.3, 50.8 | 74.9 | n.c. | n.d. |
| | 30:DPPC:NaCl | | | | | | | | | |
| 189084 | 15:25:58:2 | t = 0 | | A-T2 | 3.99 | 36.9 | 39.7, 54.6 | n.c. | n.c. | 162 |
| | ZolmitriptanFB:SD- | 40° C. | 1 Mo. | A-T2 | 2.79 | 36.8 | 39.1, 56.4 | 72.5 | n.c. | n.d. |
| | 30:DPPC:NaCl | | | | | | | | | |
| 189085 | 15:5:78:2 | t = 0 | | A-T2 | 3.17 | 36.8 | 39.4, 59.7 | n.c. | n.c. | n.d. |
| | ZolmitriptanFB:SD- | 40° C. | 1 Mo. | A-T2 | 2.94 | 36.9 | 39.2, 59.5 | n.c. | n.c. | n.d. |
| | 30:DPPC:NaCl | | | | | | | | | |

### Example 10

### Evaluation of Varied Aq:Org Ratios, Outlet Temperature, and Aircap Configurations

This evaluation was conducted to compare two variations—(i) varied air cap configurations with varied Aqueous:Organic ratios at a higher outlet temperature, and (ii) varied outlet temperatures with same air-cap configurations and Aqueous:Organic ratios. Both SD-30 based and L-leucine carrier systems were evaluated. Process parameters for these runs were summarized in Table 22.

TABLE 22

| | | Evaluation 1 | | Evaluation 2 | |
|---|---|---|---|---|---|
| | | Changing Aq:Org | | Changing | |
| Evaluation | | ratio and aircap | | outlet temp | |
| Parameters | Total solid concentration (g/L) | 4 | | 4 | |
| | Inlet Temperature (° C.) | 103 | | 103 | 79 |
| | Outlet Temperature (° C.) | 60 | | 60 | 44 |
| | Drying Gas Rate (kg/hr) | 100 | 128 | 128 | 118 |

| | Atomization Gas Flow Rate (g/min) | 22 | | 22 |
|---|---|---|---|---|
| | Aqueous Flow (mL/min) | 8 | 16 | 16 |
| | Organic flow (mL/min) | 32 | 24 | 24 |
| | Secondary drying gas flow (kg/hr) | 0 | | 0 |
| | Secondary drying gas temperature(° C.) | 0 | | 0 |
| | Product filter purge gas flow (scfh) | 15 | | 15 |
| Atomization | Air cap | 67-6-20-150 | 67147 | 67147 |

| | | | |
|---|---|---|---|
| nozzle | Fluid cap | 2850 | 2850 |

Table 23 summarizes the spray drying runs that were performed as a part of this evaluation.

TABLE 23

| | | | | Outlet |
|---|---|---|---|---|
| | | | | temp |
| Active | Carriers | Aq:Org ratio | Air Cap/Fluid cap | (° C.) |
| 15% | SD-30 (65%) + | 20:80 | 67-6-20-150/2850 | 60 |
| Zolmitriptan | DPPC (18%) + | 40:60 | 67147/2850 | 60 |
| | NaCl (2%) | | | 44 |
| | L-leu (65%) + | 20:80 | 67-6-20-150/2850 | 60 |
| | DPPC (18%) + | 40:60 | 67147/2850 | 60 |
| | NaCl (2%) | | | 44 |

Powders filled into capsules and packaged into blisters were placed on stability at 20 and 40° C. for a period of 1 month. Tables 24 and 25 list the aerosol property stability measurements for SD-30 based formulations and leucine base formulations respectively, and Table 26 lists the solid state stability behavior for SD-30 and leucine based formulations.

TABLE 24

| | | | | VMGD | ρBulk | ρTap | FPF |
|---|---|---|---|---|---|---|---|
| Lot # | Formulation | Condition | | (um) | (g/cc) | (g/cc) | (%) |
| 189112 | 15:65:18:2 | t = 0 | | 6.3 | 0.05 | 0.07 | 70 |
| | Zolmitriptan:SD- | 20° C. | 2 Wk. | 4.9 | NT | NT | 74 |
| | 30:DPPC:NaCl | | 1 Mo. | NT | NT | NT | 74 |
| | (6-hole air cap, | 40° C. | 2 Wk. | 4 | NT | NT | 74 |
| | 60 C. outlet) | | 1 Mo. | NT | NT | NT | 78 |
| 189103 | 15:65:18:2 | t = 0 | | 4.7 | 0.05 | 0.08 | 76 |
| | Zolmitriptan:SD- | 20° C. | 2 Wk. | 4.2 | NT | NT | 74 |
| | 30:DPPC:NaCl | | 1 Mo. | NT | NT | NT | 73 |
| | (1-hole air cap, | 40° C. | 2 Wk. | 4.8 | NT | NT | 76 |
| | 60 C. outlet) | | 1 Mo. | NT | NT | NT | 79 |
| 189104 | 15:65:18:2 | t = 0 | | 4.0 | 0.06 | 0.08 | 81 |
| | Zolmitriptan:SD- | 20° C. | 2 Wk. | 3.6 | NT | NT | 80 |
| | 30:DPPC:NaCl | | 1 Mo. | NT | NT | NT | 79 |
| | (1-hole air cap, | 40° C. | 2 Wk. | 4.0 | NT | NT | 71 |
| | 44 C. outlet) | | 1 Mo. | NT | NT | NT | 83 |

TABLE 25

| | | | | VMGD | pBulk | pTap | FPF |
|---|---|---|---|---|---|---|---|
| Lot # | Formulation | Condition | | (um) | (g/cc) | (g/cc) | (%) |
| 189113 | 15:65:18:2 | t = 0 | | 6 | 0.09 | 0.13 | 64 |
| | Zolmitriptan:L- | 20° C. | 2 Wk. | 7.5 | NT | NT | 62 |
| | leu:DPPC:NaCl | | 1 Mo. | NT | NT | NT | 65 |
| | (6-hole air cap, | 40° C. | 2 Wk. | 8.1 | NT | NT | 56 |
| | 60 C. outlet) | | 1 Mo. | NT | NT | NT | 61 |
| 189105 | 15:65:18:2 | t = 0 | | 2.7 | 0.07 | 0.13 | 81 |
| | Zolmitriptan:L- | 20° C. | 2 Wk. | 2.9 | NT | NT | 84 |
| | leu:DPPC:NaCl | | 1 Mo. | NT | NT | NT | 85 |
| | (1-hole air cap, | 40° C. | 2 Wk. | 3.3 | NT | NT | 82 |
| | 60 C. outlet) | | 1 Mo. | NT | NT | NT | 77 |
| 189106 | 15:65:18:2 | t = 0 | | 2.9 | 0.06 | 0.11 | 83 |
| | Zolmitriptan:L- | 20° C. | 2 Wk. | 3.1 | NT | NT | 83 |
| | leu:DPPC:NaCl | | 1 Mo. | NT | NT | NT | 86 |
| | (1-hole air cap, | 40° C. | 2 Wk. | 3.1 | NT | NT | 83 |
| | 44 C. outlet) | | 1 Mo. | NT | NT | NT | 81 |

Overall, the gPSD of the SD-30 based formulations were relatively higher than the leucine based formulations. Between the different SD-30 based formulations, the gPSD was observed to stay constant over the course of changing the aircap configuration, solvent ratios, and outlet temperatures. However, between the different leucine based formulations, the gPSD was higher for the powder produced using a 6-hole nozzle as compared to the ones produced using the single hole nozzle at both outlet temperatures.

The FPF<5.6 of both SD-30 and leucine powders produced at an outlet temperature of 44 C was much higher than the ones produced at 60° C. with both 6-hole air cap (with 20:80 Aq:Org ratio) and a 1-hole air cap (with 40:60 Aq:Org ratio). For the powders produced at a 60° C. outlet with a single hole nozzle and an Aq:Org ratio of 40:60, the leucine based formulation has a higher FPF<5.6 than the corresponding SD-30 based formulation. For the powders produced at a 60° C. outlet with a six hole nozzle and at an Aq:Org ratio of 20:80, the SD-30 based formulations has a higher FPF<5.6 than the corresponding L-Leu based formulation.

TABLE 26

| | | | | | | | | | Recryst. | |
|---|---|---|---|---|---|---|---|---|---|---|
| | | | | | TGA-120 | Low T1 | Low T2 | Recryst. | ΔH | Melt |
| Batch | Formulation | Condition | | XRPD | (%) | (° C.) | (° C.) | (° C.) | (J/g) | (° C.) |
| 189112 | 30:50:18:2 | t = 0 | | A-T2 | 3.31 | 38.5 | 40.6 | 69.4 | 1.86 | 148.7, 167.2 |
| | Zolmitriptan:SD- | 20° C. | 2 Wk. | A-T2 | 2.62 | 39.1 | 41.6, 44.3 | 69.8 | 3.68 | 160 |
| | 30:DPPC:NaCl | | 1 Mo. | A-T2 | 2.9 | 38.6 | 41.2, 44.3 | 66.9 | 3.17 | 161 |
| | (6-hole air cap, | 40° C. | 2 Wk. | A-T2 | 2.5 | 37.4 | 40.9 | 55.2, 64.8, 81.8 | n.c. | n.d. |
| | 60° C. outlet) | | 1 Mo. | A-T2 | 2.7 | 38.5 | 40.4 | 55.7, 65.0, 85.8 | n.c. | n.d. |
| 189103 | 30:50:18:2 | t = 0 | | A-T2 | 1.64 | 37.9 | 39.8, 45.7 | 77.6 | n.c. | n.d. |
| | Zolmitriptan:SD- | 20° C. | 2 Wk. | A-T2 | 2.2 | 39.2 | 42 | 69.4 | 4.07 | n.d. |
| | 30:DPPC:NaCl | | 1 Mo. | A-T2 | 2.71 | 37.5 | 40.6 | 66.8 | 5.69 | 157 |
| | (1-hole air cap, | 40° C. | 2 Wk. | A-T2 | 2.29 | 39.8 | 42.1, 52.7 | 80.3 | n.c. | n.d. |
| | 60° C. outlet) | | 1 Mo. | A-T2 | 2.95 | 37.7 | 39.2, 51.6 | 81.2 | n.c. | n.d. |
| 189104 | 30:50:18:2 | t = 0 | | A-T2 | 1.46 | 40.7 | 42.6, 45.2 | 71.6 | 6.35 | n.d. |
| | Zolmitriptan:SD- | 20° C. | 2 Wk. | A-T2 | 1.99 | 39.6 | 41.8 | 66.8 | 3.81 | n.d. |
| | 30:DPPC:NaCl | | 1 Mo. | A-T2 | 2.95 | 37.8 | 40.5, 50.9 | 66.8 | 6.32 | n.d. |
| | (1-hole air cap, | 40° C. | 2 Wk. | A-T2 | 2.54 | 39.2 | 41.8, 53.1 | 82.5 | n.c. | n.d. |
| | 44 C. outlet) | | 1 Mo. | A-T2 | 2.94 | 37.7 | 39.8, 51.9 | 81.2 | 3.24 | n.d. |
| 189113 | 30:50:18:2 | t = 0 | | PC-T2 | 0.3 | 36.0 | 42.6, 60.5 | 107.3 | 0.21 | n.d. |
| | Zolmitriptan:L- | 20° C. | 2 Wk. | PC-T2 | 0.5 | 34.4 | 51.9, 69.2 | 98.2, 155.1 | 0.54, n.c. | n.d. |
| | leu:DPPC:NaCl | | 1 Mo. | PC-T2 | 0.73 | 36.8 | 53.4, 69.4 | 89.8, 166.3 | n.c., n.c. | n.d. |
| | (6-hole air cap, | 40° C. | 2 Wk. | PC-T2 | 0.65 | 36.0 | 54.2, 69.1 | 92.7, 145.0 | n.c., 0.85 | n.d. |
| | 60° C. outlet) | | 1 Mo. | PC-T2 | 0.68 | 39.8 | 55.6, 69.2 | 94.6, 159.0 | n.c., n.c. | n.d. |
| 189105 | 30:50:18:2 | t = 0 | | PC-T2 | 0.41 | 36.4 | 52.1, 68.1 | 105.6, 147.2 | 0.62, 1.40 | n.d. |
| | Zolmitriptan:L- | 20° C. | 2 Wk. | PC-T2 | 0.5 | 37.9 | 52.4, 68.9 | 99.9 | 0.98 | n.d. |
| | leu:DPPC:NaCl | | 1 Mo. | PC-T2 | 0.97 | 33.7 | 53.1, 68.2 | 87.2 | 2.17 | n.d. |
| | (1-hole air cap, | 40° C. | 2 Wk. | PC-T2 | 0.67 | 34.8 | 54.2, 69.1 | 96.3 | n.c. | n.d. |
| | 60° C. outlet) | | 1 Mo. | PC-T2 | 0.85 | 36.6 | 54.3, 69.0 | 93 | n.c. | n.d. |
| 189106 | 30:50:18:2 | t = 0 | | PC-T2 | 0.35 | 41.7 | 54.5, 68.3 | 105.1, 160.1 | 0.32, n.c. | n.d. |
| | Zolmitriptan:L- | 20° C. | 2 Wk. | PC-T2 | 0.58 | 40.8 | 55.6, 68.7 | 97.7, 135.5 | 1.40, n.c. | n.d. |
| | leu:DPPC:NaCl | | 1 Mo. | PC-T2 | 1.03 | 35.7 | 55.1, 68.3 | 89.3 | n.c. | n.d. |
| | (1-hole air cap, | 40° C. | 2 Wk. | PC-T2 | 0.83 | 38.8 | 54.6, 69.1 | 94.1 | n.c. | n.d. |
| | 44 C. outlet) | | 1 Mo. | PC-T2 | 0.94 | 36.1 | 54.5, 69.0 | 90.7 | n.c. | n.d. |

### Example 11

### Evaluation of Collection Vessel Temperature and Sample Uniformity Over a Run

A set of experiments were performed utilizing Formulation A to evaluate two parameters, these being (1) the uniformity of powder produced over a 5 hour Zolmitriptan spray drying run and (2) the effect of collection vessel temperature on the aerosol and solid state stability of the spray dried Zolmitriptan formulation. The uniformity of powders was analyzed over four different collection vessel temperatures: 2° C., 20° C., 40° C., and 60° C. Four samples are analyzed, these being: (i) beginning fraction (taken after 1 hour), (ii) middle fraction (taken after 3 hours), (iii) end fraction (taken after 5 hours), and (iv) a composite sample, which is a composite of the first three fractions. Apart from the collection vessel temperatures, the parameters listed in Table 27 were used for spray drying all of the formulations listed below. Powders collected during these evaluations were filled into size 00 capsules (Quali-V, color HP OP White 8), packaged in Aluminum pouches and placed on stability.

TABLE 27
Spray-drying parameters utilized for Example 11.

| | | Value |
|---|---|---|
| Parameters | Total solid concentration (g/L) | 4 |
| | Inlet Temperature (° C.) | 103 |
| | Outlet Temperature (° C.) | 60 |
| | Drying Gas Rate (kg/hr) | 128 |
| | Atomization Gas Flow Rate (g/min) | 22 |
| | Aqueous Flow (mL/min) | 16 |
| | Organic flow (mL/min) | 24 |
| | Product filter purge gas flow (scfh) | 15 |
| Atomization | Air cap | 67147 |
| nozzle | Fluid cap | 2850 |

Aerosol data over the course of the stability evaluation for the case of a collection vessel temperature of 20° C. is shown in Table 28. Values for FPF<5.6 were observed to be consistent between all four samples, both at t=0 and over the course of the stability evaluation.

TABLE 28
FPF <5.6 stability data for 15% Zolmitriptan (Collection vessel
temp = 20° C.)

| | | | | | FPF <5.6 |
|---|---|---|---|---|---|
| Lot # | Conditions | Fraction | Condition | | (%) |
| 189150 | 15:65:18:2 | Fraction 1 | t = 0 | | 78 |
| | Zolmitriptan:L- | (Beginning) | 20° C. | 1 Mo. | 83 |
| | leu:DPPC:NaCl | | 40° C. | 1 Mo. | 77 |
| | (Collection | Fraction 2 | t = 0 | | 83 |
| | vessel temp = | (Middle) | 20° C. | 1 Mo. | 83 |
| | 20 C.) | | 40° C. | 1 Mo. | 76 |

| Fraction 3 | t = 0 | | 79 |
|---|---|---|---|
| (End) | 20° C. | 1 Mo. | 84 |
| | 40° C. | 1 Mo. | 77 |

| | | |
|---|---|---|
| Composite | t = 0 | 79 |

| | | |
|---|---|---|
| 20° C. | 2 Wk. | 76 |
| | 1 Mo. | 83 |
| 40° C. | 2 Wk. | 80 |
| | 1 Mo. | 75 |

Solid state characterization data for all four samples collected at 20° C. are listed in Table 29.

TABLE 29
Solid state stability data for 15% Zolmitriptan formulation (Collection vessel temp = 20° C.)

| | | | | TGA-120 | Low T 1 | Low T 2 | Recryst. | Melt |
|---|---|---|---|---|---|---|---|---|
| Formulation | Fraction | Condition | | (%) | (° C.) | (° C.) | (° C.) | (° C.) |
| 15:65:18:2 | Fraction 1 | t = 0 | | 0.35 | 31.6 | 53.5, 68.7 | 109.2, 156.9 | n.d. |
| Zolmitriptan:L- | (Beginning) | 20 C. | 1 Mo. | 0.55 | 42.1 | 49.0, 68.5 | n.c. | n.d. |
| leu:DPPC:NaCl | | 40 C. | 1 Mo. | 0.56 | 40.1 | 53.0, 69.5 | 140.5 | n.d. |
| (Collection vessel | Fraction 2 | t = 0 | | 0.53 | 37.9 | 51.2, 68.9 | 106.7, 133.8 | n.d. |
| temp = 20° C.) | (Middle) | 20 C. | 1 Mo. | 0.57 | 41.8 | 49.2, 68.5 | 101.5, 133.8 | n.d. |
| (Lot # 189150) | | 40 C. | 1 Mo. | 0.48 | 40.3 | 53.5, 69.5 | 136.3 | n.d. |
| | Fraction 3 | t = 0 | | 0.45 | 35.0 | 51.4, 68.8 | 105.0, 140.7 | n.d. |
| | (End) | 20 C. | 1 Mo. | 0.48 | 42.3 | 49.1, 68.6 | 100.9, 131.1 | n.d. |
| | | 40 C. | 1 Mo. | 0.55 | 41.4 | 53.2, 69.4 | 137.1 | n.d. |

| | | | | | | |
|---|---|---|---|---|---|---|
| Composite | t = 0 | 0.46 | 36.2 | 51.3, 68.6 | 105.0, 138.7 | n.d. |

| | | | | | | |
|---|---|---|---|---|---|---|
| 20 C. | 2 Wk. | 0.41 | 41.9 | 49.2, 68.8 | 138.7 | n.d. |
| | 1 Mo. | 0.49 | 41.7 | 49.1, 68.4 | 102.7, 135.8 | n.d. |
| 40 C. | 2 Wk. | 0.55 | 38.0 | 52.5, 68.9 | 138.2 | n.d. |
| | 1 Mo. | 0.50 | 41.1 | 52.3, 68.9 | 138.7 | n.d. |

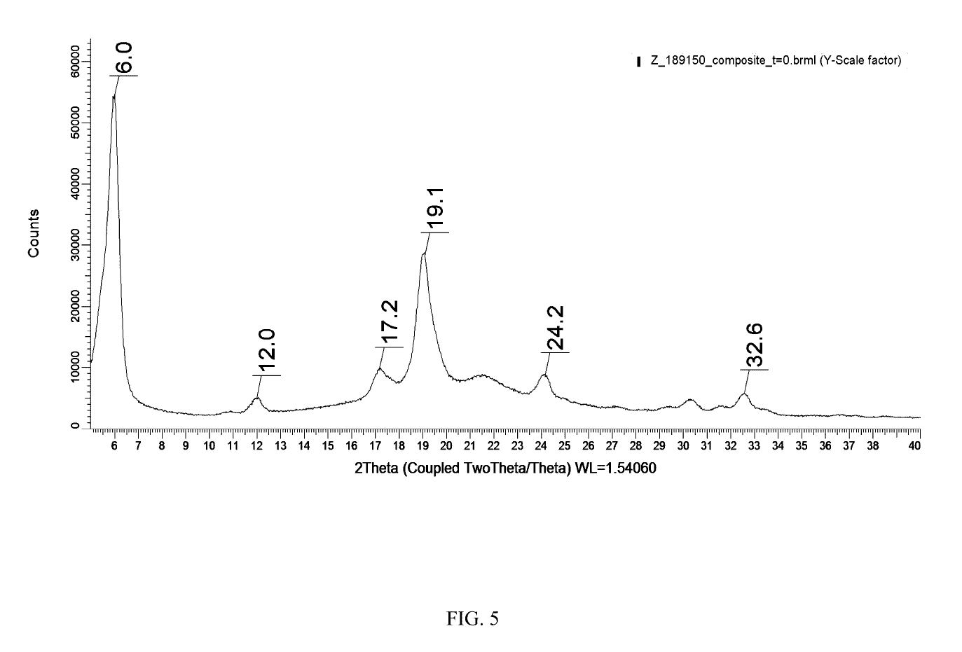

All fractions were essentially equivalent at t=0, 2 week & 1 month time points and storage conditions. XRPD showed all lots and fractions equivalent, partially crystalline with all diffraction peaks attributed to the excipients leucine and DPPC. A representative XRPD diffractogram for Formulation A lot 189150 at t=0 is shown in FIG. 5 with the peak positions identified (PC-T2 pattern) as characteristic of this formulation. No detectable changes in the diffractograms were observed on stability. TGA showed consistent weight loss across lots and fractions with ˜0.5% loss up to 120° C. DSC for all lots showed broad exothermic transitions in the ˜90-155 C region indicating thermal recrystallization.

Aerosol data over the course of the stability evaluation for the case of a collection vessel temperature of 40° C. is shown in Table 30. Values for FPF<5.6 were observed to be consistent between all four samples, both at t=0 and over the course of the stability evaluation.

TABLE 30
FPF <5.6 stability data for 15% Zolmitriptan (Collection vessel
temp = 40° C.)

| | | | | | FPF <5.6 |
|---|---|---|---|---|---|
| Lot # | Conditions | Fraction | Condition | | (%) |
| 189152 | 15:65:18:2 | Fraction 1 | t = 0 | | 78 |
| | Zolmitriptan:L- | (Beginning) | 20° C. | 1 Mo. | 83 |
| | leu:DPPC:NaCl | | 40° C. | 1 Mo. | 74 |
| | (Collection vessel | Fraction 2 | t = 0 | | 82 |
| | temp = 40° C.) | (Middle) | 20° C. | 1 Mo. | 86 |
| | | | 40° C. | 1 Mo. | 76 |

| Fraction 3 | t = 0 | | 71 |
|---|---|---|---|
| (End) | 20° C. | 1 Mo. | 84 |
| | 40° C. | 1 Mo. | 78 |

| | | |
|---|---|---|
| Composite | t = 0 | 79 |

| | | |
|---|---|---|
| 20° C. | 2 Wk. | 80 |
| | 1 Mo. | 83 |
| 40° C. | 2 Wk. | 80 |
| | 1 Mo. | 75 |

Solid state characterization data for all four samples collected at 40° C. are listed in Table 31.

TABLE 31
Solid state stability data for 15% Zolmitriptan (Collection vessel temp = 40 C.)

| | | | | TGA-120 | Low T 1 | Low T 2 | Recryst. | Melt |
|---|---|---|---|---|---|---|---|---|
| Conditions | Fraction | Condition | | (%) | (° C.) | (° C.) | (° C.) | (° C.) |
| 15:65:18:2 | Fraction 1 | t = 0 | | 0.38 | 38.8 | 51.8, 68.4 | 113.4, 142.6 | n.d. |
| Zolmitriptan:L- | (Beginning) | 20° C. | 1 Mo. | 0.57 | 41.3 | 50.5, 68.6 | 90-150 (range) | n.d. |
| leu:DPPC:NaCl | | 40° C. | 1 Mo. | 0.56 | 40.2 | 52.7, 69.4 | 136.3 | n.d. |
| (Collection vessel | Fraction 2 | t = 0 | | 0.32 | 38.1 | 52.2, 68.3 | 100-150 (range) | n.d. |
| temp = 40° C.) | (Middle) | 20° C. | 1 Mo. | 0.57 | 42.0 | 51.1, 68.5 | 101.7, 128.1 | n.d. |
| (Lot # 189152) | | 40° C. | 1 Mo. | 0.66 | 39.8 | 53.3, 69.3 | 133.3 | n.d. |
| | Fraction 3 | t = 0 | | 0.39 | 38.2 | 52.5, 68.3 | 90-140 (range) | n.d. |
| | (End) | 20° C. | 1 Mo. | 0.54 | 40.7 | 50.3, 68.5 | 100.4, 133.3 | n.d. |
| | | 40° C. | 1 Mo. | 0.57 | 39.0 | 53.2, 69.3 | 134.1 | n.d. |

| | | | | | | |
|---|---|---|---|---|---|---|
| Composite | t = 0 | 0.38 | 39.4 | 52.5, 68.4 | 90-150 (range) | n.d. |

| | | | | | | |
|---|---|---|---|---|---|---|
| 20° C. | 2 Wk. | 0.55 | 39.9 | 51.6, 69.0 | 90-150 (range) | n.d. |
| | 1 Mo. | 0.63 | 41.5 | 50.2, 68.4 | 101.2, 130.5 | n.d. |
| 40° C. | 2 Wk. | 0.61 | 37.5 | 52.8, 69.1 | 90-150 (range) | n.d. |
| | 1 Mo. | 0.57 | 37.5 | 51.7, 68.9 | 138.8 | n.d. |

Within each lot, all fractions were essentially equivalent at t=0, 2 week & 1 month time points and storage conditions. XRPD showed all lots and fractions to be equivalent, partially crystalline with all diffraction peaks attributed to excipients. No detectable changes in the diffractograms were observed on stability. TGA showed consistent weight loss across lots and fractions with ˜0.5% loss up to 120° C. DSC for all lots showed broad exothermic transitions in the ˜90-155 C region indicating thermal recrystallization.

Aerosol data over the course of the stability evaluation for the case of a collection vessel temperature of 60° C. is shown in Table 32. Values for FPF<5.6 were observed to be consistent between all four samples, both at t=0 and over the course of the stability evaluation.

TABLE 32
FPF <5.6 stability data for 15% Zolmitriptan (Collection vessel
temp = 60° C.)

| | | | | | FPF <5.6 |
|---|---|---|---|---|---|
| Lot # | Conditions | Fraction | Condition | | (%) |
| 189154 | 15:65:18:2 | Fraction 1 | t = 0 | | 75 |
| | Zolmitriptan:L- | (Beginning) | 20° C. | 1 Mo. | 79 |
| | leu:DPPC:NaCl | | 40° C. | 1 Mo. | 67 |
| | (Collection | Fraction 2 | t = 0 | | 81 |
| | vessel temp = | (Middle) | 20° C. | 1 Mo. | 82 |
| | 60° C.) | | 40° C. | 1 Mo. | 72 |

| Fraction 3 | t = 0 | | 79 |
|---|---|---|---|
| (End) | 20° C. | 1 Mo. | 84 |

| | | |
|---|---|---|
| 40° C. | 1 Mo. | 73 |

| | | |
|---|---|---|
| Composite | t = 0 | 78 |

| | | |
|---|---|---|
| 20° C. | 2 Wk. | 87 |
| | 1 Mo. | 82 |
| 40° C. | 2 Wk. | 70 |
| | 1 Mo. | 73 |

Solid state characterization data for all four samples collected at 60° C. are listed in Table 33.

TABLE 33
Solid state stability data for 15% Zolmitriptan (Collection vessel temp = 60° C.)

| | | | | TGA-120 | Low T 1 | Low T 2 | Recryst. | Melt |
|---|---|---|---|---|---|---|---|---|
| Conditions | Fraction | Condition | | (%) | (° C.) | (° C.) | (° C.) | (° C.) |
| 15:65:18:2 | Fraction 1 | t = 0 | | 0.388 | 37.1 | 47.9, 68.0 | 90-150 (range) | n.d. |
| Zolmitriptan:L- | (Beginning) | 20° C. | 1 Mo. | 0.65 | 41.6 | 48.3, 68.0 | 101.0, 134.0 | n.d. |
| leu:DPPC:NaCl | | 40° C. | 1 Mo. | 0.60 | 37.2 | 49.2, 68.4 | 132.3 | n.d. |
| (Collection vessel | Fraction 2 | t = 0 | | 0.28 | 38.0 | 47.0, 67.5 | 100-150 (range) | n.d. |
| temp = 60° C.) | (Middle) | 20° C. | 1 Mo. | 0.65 | 41.9 | 48.2, 67.8 | 101.4, 129.2 | n.d. |
| (Lot # 189154) | | 40° C. | 1 Mo. | 0.56 | 37.2 | 49.0, 68.3 | 132.9 | n.d. |
| | Fraction 3 | t = 0 | | 0.27 | 37.2 | 47.6, 68.0 | 90-155 (range) | n.d. |
| | (End) | 20° C. | 1 Mo. | 0.53 | 41.8 | 47.8, 67.8 | 100-150 (range) | n.d. |
| | | 40° C. | 1 Mo. | 0.46 | 35.0 | 48.7, 68.1 | 110-150 (range) | n.d. |

| | | | | | | |
|---|---|---|---|---|---|---|
| Composite | t = 0 | 0.44 | 41.5 | 47.5, 68.0 | 90-150 (range) | n.d. |

| | | | | | | |
|---|---|---|---|---|---|---|
| 20° C. | 2 Wk. | 0.54 | 41.1 | 48.2, 67.9 | 90-150 | n.d. |
| | 1 Mo. | 0.54 | 40.5 | 48.2, 67.8 | 102.2, 133.7 | n.d. |
| 40° C. | 2 Wk. | 0.63 | 36.9 | 49.1, 69.2 | 133 (110-140) | n.d. |
| | 1 Mo. | 0.59 | 36.8 | 49.1, 68.4 | 135.1 | n.d. |

Within each lot, all fractions were essentially equivalent at t=0, 2 week & 1 month time points and storage conditions. XRPD showed all lots and fractions equivalent, partially crystalline with all diffraction peaks attributed to excipients. No detectable changes in the diffractograms were observed on stability. TGA showed consistent weight loss across lots and fractions with ˜0.5% loss up to 120° C. DSC for all lots showed broad exothermic transitions in the ˜90-155 C region indicating thermal recrystallization.

Aerosol data over the course of the stability evaluation for the case of a collection vessel temperature of 2° C. is shown in Table 31. Values for FPF<5.6 were observed to be consistent between all four samples at t=0 However, after 1 month at 40° C., all samples show a slight drop in the FPF<5.6 values. Table 34 below lists the FPF<5.6 values from each sample collected when the collection vessel was maintained at 2° C.

TABLE 34
FPF <5.6 data for 15% Zolmitriptan (Collection
vessel temp = 2° C.)

| | | | | | FPF <5.6 |
|---|---|---|---|---|---|
| Lot # | Conditions | Fraction | Condition | | (%) |
| 189156 | 15:65:18:2 | Fraction 1 | t = 0 | | 79 |
| | Zolmitriptan:L- | (Beginning) | 20° C. | 1 Mo. | 79 |
| | leu:DPPC:NaCl | | 40° C. | 1 Mo. | 65 |
| | (Collection vessel | Fraction 2 | t = 0 | | 77 |
| | temp = 2 C.) | (Middle) | 20° C. | 1 Mo. | 80 |
| | | | 40° C. | 1 Mo. | 62 |

| Fraction 3 | t = 0 | | 80 |
|---|---|---|---|
| (End) | 20° C. | 1 Mo. | 78 |
| | 40° C. | 1 Mo. | 66 |

| | | |
|---|---|---|
| Composite | t = 0 | 78 |

| | | |
|---|---|---|
| 20° C. | 2 Wk. | 80 |
| | 1 Mo. | 79 |
| 40° C. | 2 Wk. | 74 |
| | 1 Mo. | 66 |

Solid state characterization data for all four samples collected at 60° C. are listed in Table 35.

TABLE 35
Solid state stability data for 15% Zolmitriptan (Collection vessel temp = 2 C.)

| | | | | TGA-120 | Low T 1 | Low T 2 | Recryst. | Melt |
|---|---|---|---|---|---|---|---|---|
| Conditions | Fraction | Condition | | (%) | (° C.) | (° C.) | (° C.) | (° C.) |
| 15:65:18:2 | Fraction 1 | t = 0 | | 0.54 | 35.7 | 48.7, 68.4 | 96.1, 133.4 | n.d. |
| Zolmitriptan:L- | (Beginning) | 20° C. | 1 Mo. | 0.42 | 42.6 | 49.2, 68.2 | 98.6, 126.4 | n.d. |
| leu:DPPC:NaCl | | 40° C. | 1 Mo. | 0.87 | 36.3 | 48.6, 68.8 | 131.4 | n.d. |
| (Collection vessel | Fraction 2 | t = 0 | | 0.46 | 36.9 | 49.5, 68.5 | 95.5, 125.5 | n.d. |
| temp = 2° C.) | (Middle) | 20° C. | 1 Mo. | 0.51 | 38.7 | 49.0, 68.2 | 96.6, 132.8 | n.d. |
| (Lot #189156) | | 40° C. | 1 Mo. | 0.81 | 34.8 | 49.0, 68.9 | 127.8 | n.d. |
| | Fraction 3 | t = 0 | | 0.57 | 36.9 | 49.9, 68.8 | 98.2, 128.4 | n.d. |
| | (End) | 20° C. | 1 Mo. | 0.59 | 38.7 | 49.3, 68.2 | 98.2, 126.5 | n.d. |
| | | 40° C. | 1 Mo. | 0.64 | 35.4 | 48.7, 68.7 | 133.4 | n.d. |

| | | | | | | |
|---|---|---|---|---|---|---|
| Composite | t = 0 | 0.54 | 36.2 | 49.3, 68.6 | 96.5, 132.5 | n.d. |

| | | | | | | |
|---|---|---|---|---|---|---|
| 20° C. | 2 Wk. | 0.70 | 40.9 | 49.8, 68.4 | 153 (130-170) | n.d. |
| | 1 Mo. | 0.61 | 39.3 | 49.4, 68.4 | 95.7, 130.1 | n.d. |
| 40° C. | 2 Wk. | 0.46 | 35.4 | 49.3, 68.7 | 129 (115-140) | n.d. |
| | 1 Mo. | 0.80 | 34.0 | 48.8, 68.7 | 129.5 | n.d. |

Within each lot, all fractions were essentially equivalent at t=0, 2 week & 1 month time points and storage conditions. XRPD showed all lots and fractions equivalent, partially crystalline with all diffraction peaks attributed to excipients (PC-T2 pattern). No detectable changes in the diffractograms were observed on stability. TGA showed consistent weight loss across lots and fractions with ˜0.5% loss up to 120° C. DSC for all lots showed broad exothermic transitions in the ˜90-155 C region indicating thermal recrystallization.

### Example 12

### Evaluation of Zolmitriptan Formulations Produced at Different Total Solid Concentrations

A set of experiments was performed to examine the effects of increasing the total solid concentration of the spray dried solution. Two air caps were evaluated, these being a single hole (67147) and a 6-hole (67-6-20-150). Powders spray dried during these evaluations were filled into size 00 capsules (Quali-V, color HP OP White 8), packaged in Aluminum pouches and placed on stability. The spray-drying parameters shown in Table 36 were used to produce the powders utilizing the single hole nozzle.

TABLE 36

| | | Value |
|---|---|---|
| Parameters | Total solid concentration (g/L) | 2, 4, 6, 8, 10, 12 |
| | Inlet Temperature (° C.) | 103 |
| | Outlet Temperature (° C.) | 60 |
| | Drying Gas Rate (kg/hr) | 128 |
| | Atomization Gas Flow Rate (g/min) | 22 |
| | Aqueous Flow (mL/min) | 16 |
| | Organic flow (mL/min) | 24 |
| | Secondary drying gas flow (kg/hr) | 0 |
| | Secondary drying gas temperature | 0 |
| | (° C.) | |
| | Product filter purge gas flow (scfh) | 15 |
| Atomization | Air cap | 67147 |
| nozzle | Fluid cap | 2850 |

Aerosol and solid-state characterization results are shown in Tables 37 and 38 for the case of the single hole nozzle. As the total solid concentration was increased, the particle size was observed to increase and the density was observed to decrease (FPFs were relatively insensitive to concentration change).

TABLE 37
Aerosol data for 15% Zolmitriptan produced with varied total solid concentrations

| | Total solid | | | VMGD | ρBulk | ρTap | FPF <5.6 |
|---|---|---|---|---|---|---|---|
| Lot # | concentration | Condition | | (um) | (g/cc) | (g/cc) | (%) |
| 189171 | 15% Zolmitriptan | t = 0 | | 2.1 | NT | NT | 76 |
| | (2 g/L) | 20° C. | 1 Mo. | NT | NT | NT | 80 |
| | | 40° C. | 2 Wk. | NT | NT | NT | 74 |
| | | | 1 Mo. | NT | NT | NT | 68 |
| 189168 | 15% Zolmitriptan | t = 0 | | 2.4 | 0.132 | 0.21 | 85 |
| | (4 g/L) | 20° C. | 1 Mo. | NT | NT | NT | 83 |
| | | 40° C. | 2 Wk. | NT | NT | NT | 78 |
| | | | 1 Mo. | NT | NT | NT | 73 |
| 189170 | 15% Zolmitriptan | t = 0 | | 3 | 0.104 | 0.16 | 77 |
| | (6 g/L) | 20° C. | 1 Mo. | NT | NT | NT | 80 |
| | | 40° C. | 2 Wk. | NT | NT | NT | 71 |
| | | | 1 Mo. | NT | NT | NT | 72 |
| 189169 | 15% Zolmitriptan | t = 0 | | 3.3 | 0.82 | 0.15 | 75 |
| | (8 g/L) | 20° C. | 1 Mo. | NT | NT | NT | 73 |
| | | 40° C. | 2 Wk. | NT | NT | NT | 69 |
| | | | 1 Mo. | NT | NT | NT | 68 |
| 189174 | 15% Zolmitriptan | t = 0 | | 3.7 | 0.09 | 0.21 | 73 |
| | (10 g/L) | 20° C. | 1 Mo. | NT | NT | NT | 77 |
| | | 40° C. | 2 Wk. | NT | NT | NT | 75 |
| | | | 1 Mo. | NT | NT | NT | 74 |
| 189175 | 15% Zolmitriptan | t = 0 | | 4.2 | 0.07 | 0.15 | 72 |
| | (12 g/L) | 20° C. | 1 Mo. | NT | NT | NT | 82 |
| | | 40° C. | 2 Wk. | NT | NT | NT | 72 |
| | | | 1 Mo. | NT | NT | NT | 73 |

TABLE 38
Solid-state data for 15% Zolmitriptan produced with varied total solid concentrations

| | | | | | | | | MDSC |
|---|---|---|---|---|---|---|---|---|
| | Total solid | | | TGA-120 | Low T 1 | Low T 2 | Recryst. | Recryst. |
| Lot # | concentration | Condition | | (%) | (° C.) | (° C.) | (° C.) | ΔH (J/g) |
| 189171 | 15% Zolmitriptan | t = 0 | | 0.40 | 35.8 | 49.7, 66.7 | 90-140 | 1.22 |
| | (2 g/L) | 20° C. | 1 Mo. | 0.40 | 38.7 | 48.8, 67.7 | 90-140 | 3.25 |
| | | 40° C. | 2 Wk. | 0.37 | 38.9 | 49.7, 67.9 | 85-156 | 5.36 |
| | | | 1 Mo. | 0.48 | 37.2 | 49.9, 68.1 | 90-150 | 3.08 |
| 189168 | 15% Zolmitriptan | t = 0 | | 0.75 | 36.8 | 49.2, 67.6 | 90-130 | 2.85 |
| | (4 g/L) | 20° C. | 1 Mo. | 0.63 | 37.8 | 50.2, 68.8 | 90-130 | 1.34 |
| | | 40° C. | 2 Wk. | 0.39 | 43.4 | 51.4, 68.0 | minimal | 4.54 |
| | | | 1 Mo. | 0.40 | 37.1 | 52.2, 68.9 | minimal | 2.90 |
| 189170 | 15% Zolmitriptan | t = 0 | | 0.47 | 33.5 | 53.6, 67.6 | 90-130 | 5.28 |
| | (6 g/L) | 20° C. | 1 Mo. | 0.49 | 43.8 | 50.0, 68.7 | 100-160 | 8.86 |
| | | 40° C. | 2 Wk. | 0.29 | 40.7 | 51.1, 68.5 | 100-160 | 7.65 |
| | | | 1 Mo. | 0.32 | 38.4 | 52.6, 68.4 | 100-165 | 13.44 |
| 189169 | 15% Zolmitriptan | t = 0 | | 0.53 | 34.8 | 53.8, 67.7 | 90-150 | 5.70 |
| | (8 g/L) | 20° C. | 1 Mo. | 0.49 | 40.6 | 49.6, 68.5 | 100-165 | 10.15 |
| | | 40° C. | 2 Wk. | 0.30 | 43.7 | 51.0, 67.9 | 100-160 | 11.48 |
| | | | 1 Mo. | 0.46 | 38.7 | 52.8, 68.6 | 100-170 | 10.07 |
| 189174 | 15% Zolmitriptan | t = 0 | | 0.32 | 35.5 | 54.0, 68.8 | 90-150 | 9.73 |
| | (10 g/L) | 20° C. | 1 Mo. | 0.30 | 38.6 | 50.0, 68.8 | 120-165 | 8.07 |
| | | 40° C. | 2 Wk. | 0.40 | 38.9 | 51.6, 69.0 | 120-160 | 5.54 |
| | | | 1 Mo. | 0.47 | 38.7 | 53.5, 69.4 | 120-165 | 6.82 |
| 189175 | 15% Zolmitriptan | t = 0 | | 0.27 | 35.0 | 55.4, 68.4 | 90-160 | 6.03 |
| | (12 g/L) | 20° C. | 1 Mo. | 0.38 | 37.3 | 51.3, 69.1 | 100-170 | 9.50 |
| | | 40° C. | 2 Wk. | 0.35 | 42.8 | 52.4, 69.0 | 100-160 | 5.88 |
| | | | 1 Mo. | 0.49 | 40.4 | 54.7, 69.3 | 105-165 | 9.53 |

XRPD analysis showed all lots to be equivalent, partially crystalline with all diffraction peaks attributed to excipients (PC-T2 pattern). TGA showed consistent weight loss across lots with ˜0.3-0.8% loss up to 120° C. with a slightly higher weight loss with the 4 g/L lot. DSC showed generally consistent thermal events. The glass transition event is minimal given the semi-crystalline state of the material; modulated differential scanning calorimetry (MDSC) showed more equivalent glass transition intercept temperatures. The DPPC phase transitions show subtle differences with the 2 & 4 g/L versus the 6 & 8 g/L and the event at ˜67° C. increases in intensity as solids load increases. Small crystallization 90-150° C. region is consistently observed which is attributed to residual amorphous leucine (small changes in enthalpy).

Spray-drying parameters for the case of the 6-hole nozzle are shown in Table 39, with aerosol and solid-state characterization results shown in Tables 40 and 41.

TABLE 39

| | | Value |
|---|---|---|
| Parameters | Total solid concentration (g/L) | 12 |
| | Inlet Temperature (° C.) | 103 |
| | Outlet Temperature (° C.) | 60 |
| | Drying Gas Rate (kg/hr) | 128 |
| | Atomization Gas Flow Rate (g/min) | 22 |
| | Aqueous Flow (mL/min) | 16 |
| | Organic flow (mL/min) | 24 |
| | Secondary drying gas flow (kg/hr) | 0 |
| | Secondary drying gas temperature (° C.) | 0 |
| | Product filter purge gas flow (scfh) | 15 |
| Atomization | Air cap | 67-6-20-150 |
| nozzle | Fluid cap | 2850 |

As seen in Table 40, as the total solid concentration went up, the FPF<5.6 and the particle stayed consistently high, both at t=0 and over the course of the stability study. However, the FPF<5.6 was much lower than that of the powder produced using a single-hole aircap.

TABLE 40
FPF <5.6 data for 15% Zolmitriptan produced
with varied total solid concentrations

| | Total solid | | | VMGD | FPF <5.6 |
|---|---|---|---|---|---|
| Lot # | concentration | Condition | | (um) | (%) |
| 189186 | 15% Zolmitriptan with | t = 0 | | 13.6 | 68 |
| | 67-6-20-150 air cap | 20° C. | 1 Mo. | NT | 68 |
| | (4 g/L) | 40° C. | 2 Wk. | NT | 66 |
| | | | 1 Mo. | NT | 61 |
| 189187 | 15% Zolmitriptan with | t = 0 | | 11.4 | 66 |
| | 67-6-20-150 air cap | 20° C. | 1 Mo. | NT | 70 |
| | (8 g/L) | 40° C. | 2 Wk. | NT | 68 |
| | | | 1 Mo. | NT | 63 |
| 189188 | 15% Zolmitriptan with | t = 0 | | 15 | 63 |
| | 67-6-20-150 air cap | 20° C. | 1 Mo. | NT | 67 |
| | (12 g/L) | 40° C. | 2 Wk. | NT | 66 |
| | | | 1 Mo. | NT | 62 |

TABLE 41
Solid-state data for 15% Zolmitriptan produced with varied total solid concentrations

| | | | | | | | | MDSC |
|---|---|---|---|---|---|---|---|---|
| | Total solid | | | TGA-120 | Low T 1 | Low T 2 | Recryst. | Recryst. |
| Lot # | concentration | Condition | | (%) | (° C.) | (° C.) | (° C.) | ΔH (J/g) |
| 189186 | 15% Zolmitriptan | t = 0 | | 0.30 | 35.9 | 53.0, 68.1 | 90-160 | 5.46 |
| | with 67-6-20-150 | 20° C. | 1 Mo. | 0.32 | 39.9 | 50.4, 69.3 | 120-165 | 12.2 |
| | air cap (4 g/L) | 40° C. | 2 Wk. | 0.17 | 37.8 | 53.1, 69.0 | 120-160 | 7.52 |
| | | | 1 Mo. | 0.39 | 35.7 | 53.3, 68.9 | 110-160 | 17.17 |
| 189187 | 15% Zolmitriptan | t = 0 | | 0.31 | 36.1 | 53.3, 68.1 | 90-160 | 12.14 |
| | with 67-6-20-150 | 20° C. | 1 Mo. | 0.38 | 41.9 | 50.6, 69.4 | 90-165 | 12.29 |
| | air cap (8 g/L) | 40° C. | 2 Wk. | 0.24 | 40.4 | 54.9, 69.2 | 100-160 | 6.86 |
| | | | 1 Mo. | 0.26 | 41.1 | 54.3, 69.1 | 110-170 | 9.02 |
| 189188 | 15% Zolmitriptan | t = 0 | | 0.34 | 36.0 | 57.1, 68.2 | 90-160 | 7.42 |
| | with 67-6-20-150 | 20° C. | 1 Mo. | 0.29 | 42.1 | 49.9, 68.8 | 90-165 | 16.37 |
| | air cap (12 g/L) | 40° C. | 2 Wk. | 0.39 | 42.0 | 55.2, 69.2 | 100-160 | 3.46 |
| | | | 1 Mo. | 0.33 | 41.3 | 54.5, 69.1 | 95-165 | 11.96 |

XRPD analysis showed all lots to be equivalent, partially crystalline with all diffraction peaks attributed to excipients. TGA showed consistent weight loss across lots with ˜0.3-0.8% loss up to 120° C. DSC showed generally consistent thermal events. The glass transition event is minimal given the semi-crystalline state of the material. Small crystallization 90-150° C. region is consistently observed which is attributed to residual amorphous leucine (small changes in enthalpy).

### Example 13

### Chemical Stability: Evaluation of Aqueous Phase pH Adjustments

Another advantageous aspect of the invention disclosed herein is the identification of a combination of processing and packaging conditions that enable the production of powders containing amorphous zolmitriptan that are chemically stable at both ambient and accelerated temperature storage conditions to an extent required for regulatory approval of a room-temperature stable pharmaceutical product. It was surprisingly discovered that the methods and formulation parameters conventionally utilized for the preparation of room-temperature stable tablet and nasal solution zolmitriptan commercial products were not sufficient for the preparation of chemically stable dry powders for inhalation containing zolmitriptan in an amorphous state. Tablet formulations containing zolmitriptan typically contain the drug in a micronized crystalline form, with crystalline forms of drugs typically observed to show improved physical and chemical stability properties to amorphous forms of the same drug. It was discovered through our work described in this application that (i) zolmitriptan powders possessing FPFs required for inhalation could not be produced via spray-drying with zolmitriptan being present in a crystalline state and (ii) amorphous zolmitriptan in the solid-state can be susceptible to relatively rapid chemical degradation at both ambient and elevated (accelerated) temperature storage conditions.

Zolmitriptan solutions for nasal administration, in which zolmitriptan is present in solution in a highly mobile state (i.e., different than the crystalline state) have been demonstrated to be room-temperature stable when the pH of the solution is slightly acidic (in the range of pH=5). As a result, initial attempts to improve the stability of dry powders containing amorphous zolmitriptan as disclosed herein involved alterations of the pH of the aqueous phase spray-drying solution utilizing hydrochloric acid, citric acid, and ascorbic acid. Parameters listed in Appendix D were used to produce these powders. For the case of hydrochloric acid, the pH of the aqueous phase was decreased to 5.0 and 4.0 and powders were produced utilizing the processing conditions shown in Table 42.

TABLE 42

| | | Value |
|---|---|---|
| Parameters | Total solid concentration (g/L) | 4 |
| | Inlet Temperature (° C.) | 103 |
| | Outlet Temperature (° C.) | 60 |
| | Drying Gas Rate (kg/hr) | 128 |
| | Atomization Gas Flow Rate (g/min) | 22 |
| | Aqueous Flow (mL/min) | 16 |
| | Organic flow (mL/min) | 24 |
| | Secondary drying gas flow (kg/hr) | 0 |
| | Secondary drying gas temperature (° C.) | 0 |
| | Product filter purge gas flow (scfh) | 15 |
| Atomization | Air cap | 67147 |
| nozzle | Fluid cap | 2850 |

The resultant spray dried Zolmitriptan formulations were filled into size 00 capsules (Quali-V, color HP OP White 8), packaged in Aluminum pouches, and evaluated for chemical stability by storing samples at 20 C, 40 C, and 50° C. over the course of a month. Table 43 below details the FPF<5.6 and the Zolmitriptan purity values over the course of this study. As is evident from the data, surprisingly, modification of the aqueous phase pH to values utilized in known nasal zolmitriptan formulations resulted in a reduced stability of zolmitriptan in the amorphous solid state in spray-dried powders.

TABLE 43
FPF <5.6 and purity data for 15% Zolmitriptan
produced with varied HCl to adjust aqueous pH

| | 15:65:18:2 | | | | |
|---|---|---|---|---|---|
| | Zolmi:L- | | | FPF <5.6 | |
| Lot # | leu:DPPC:NaCl | Condition | | (%) | Purity |
| 224003-1 | No pH adj, aqueous | t = 0 | | 85 | NT |
| | N2 purge, Filling = | 40° C. | 1 Wk. | 84 | 99.75 |
| | 20 C., 15% RH | | 2 Wk. | NT | 98.8 |
| | | | 1 Mo. | 80 | 98 |
| | | 50° C. | 1 Wk. | 65 | 99.39 |
| | | | 2 Wk. | NT | 95.85 |
| | | | 1 Mo. | 67 | 93.88 |
| 224003-2 | 15:65:18:2 | t = 0 | | 87 | NT |
| | Zolmi:L- | 40° C. | 1 Wk. | 84 | 99.8 |
| | leu:DPPC:NaCl | | 2 Wk. | NT | 97.62 |
| | (Aq. pH = 5, | | 1 Mo. | 82 | 96.41 |
| | aqueous | 50° C. | 1 Wk. | 67 | 98.83 |
| | N2 purge, Filling = | | 2 Wk. | NT | 93.52 |
| | 20 C., 15% RH) | | 1 Mo. | 56 | 90.64 |
| 224003-3 | 15:65:18:2 | t = 0 | | 86 | NT |
| | Zolmi:L- | 40° C. | 1 Wk. | 80 | 98.27 |
| | leu:DPPC:NaCl | | 2 Wk. | NT | 96.48 |
| | (Aq. pH = 4, Yes | | 1 Mo. | 86 | 95.19 |
| | N2 purge, Filling = | 50° C. | 1 Wk. | 71 | 95.36 |
| | 20 C., 15% RH) | | 2 Wk. | NT | 91.5 |
| | | | 1 Mo. | 71 | 85.45 |

Similar to the Hydrochloric acid evaluation, two citric acid and two ascorbic acid concentrations were evaluated with similar reductions of the pH of the aqueous phase to 5.0 and 4.0. FPF. Purity data from these evaluations are shown in Table 44 for citric acid and Table 45 for ascorbic acid. The FPF<5.6 value is observed to stay consistent, but similar results were seen with respect to a decrease in purity at decreased aqueous phase pH.

TABLE 44
FPF <5.6 and purity data for 15% Zolmitriptan produced
with varied Citric acid to adjust aqueous pH.

| | | | | | Purity |
|---|---|---|---|---|---|
| Lot # | Composition | Condition | | FPF <5.6 | (%) |
| 224010 | 15% Zolmitriptan + | t = 0 | | 85 | NT |
| | 64.82% L-leu + 18% | 20° C. | 1 Mo. | 87 | NT |
| | DPPC + 2% NaCl + | 40° C. | 2 Wk. | NT | 89.87 |
| | 0.18% Citric acid | | 1 Mo. | 76 | 98.28 |
| | (pH of aq phase = 5) | 50° C. | 1 Wk. | NT | NT |
| | | | 2 Wk. | NT | 95.99 |
| | | | 1 Mo. | NT | 94.08 |
| 224011 | 15% Zolmitriptan + | t = 0 | | 82 | NT |
| | 62.50% L-leu + 18% | 20° C. | 1 Mo. | 78 | NT |
| | DPPC + 2% NaCl + | 40° C. | 2 Wk. | NT | 99.06 |
| | 2.50% Citric acid | | 1 Mo. | 74 | 98.65 |
| | (pH of aq phase = 4) | 50° C. | 1 Wk. | NT | NT |
| | | | 2 Wk. | NT | 95.95 |
| | | | 1 Mo. | NT | 94.41 |

TABLE 45
FPF <5.6 and purity data for 15% Zolmitriptan produced
with varied Ascorbic acid to adjust aqueous pH.

| | | | | | Purity |
|---|---|---|---|---|---|
| Lot # | Composition | Condition | | FPF | (%) |
| 224006 | 15% Zolmitriptan + | t = 0 | | 81 | NT |
| | 64.90% L-leu + 18% | 20° C. | 1 Mo. | 84 | NT |
| | DPPC + 2% NaCl + | 40° C. | 1 Wk. | NT | NT |
| | 0.10% Ascorbic acid | | 2 Wk. | NT | 98.58 |
| | | | 1 Mo. | 78 | 98.03 |
| | | 50° C. | 1 Wk. | NT | NT |
| | | | 2 Wk. | NT | 96.26 |
| | | | 1 Mo. | NT | 94.53 |
| 224007 | 15% Zolmitriptan + | t = 0 | | 79 | NT |
| | 64.90% L-leu + 18% | 20° C. | 1 Mo. | 79 | NT |
| | DPPC + 2% NaCl + | 40° C. | 2 Wk. | NT | 96.76 |
| | 1.00% Ascorbic acid | | 1 Mo. | 75 | 96.33 |
| | | 50° C. | 1 Wk. | NT | NT |
| | | | 2 Wk. | NT | 93.82 |
| | | | 1 Mo. | NT | 90.75 |

### Example 14

### Evaluation of Antioxidants

Since the drop in purity of Zolmitriptan in initial formulations appeared to be at least in part due to oxidation of Zolmitriptan, several antioxidants were examined for inclusion in spray-dried zolmitriptan powders to attempt to improve their chemical stability. Two commonly utilized antioxidants were examined at concentration levels that are typically used in pharmaceutical formulations, these being (i) EDTA and (ii) alpha tocopherol, with spray-drying and packaging parameters identical to those described in Chemical Stability Example 1 utilized for powder production and placement on stability. As seen from the results in Table 46, for the case of EDTA addition (values of 0.01% and 0.075%), the FPF<5.6 stayed consistent, but the purity value dropped significantly over 1 week. The study was discontinued because of the excessive drop in purity values for both EDTA concentrations.

TABLE 46
FPF <5.6 data for 15% Zolmitriptan produced
with varied EDTA as an anti-oxidant

| | | | | FPF <5.6 | Purity |
|---|---|---|---|---|---|
| Lot # | Formulation | Condition | | (%) | (%) |
| 224018 | 15% Zolmitriptan + | t = 0 | | 83 | 99.95 |
| | 64.99% L-leu + 18% | 40° C. | 1 Wk. | 85 | 96.66 |
| | DPPC + 2% NaCl + | 50° C. | 1 Wk. | 76 | 89.84 |
| | 0.01% EDTA | | | | |
| 224019 | 15% Zolmitriptan + | t = 0 | | 83 | 99.82 |
| | 64.925% L-leu + | 40° C. | 1 Wk. | 88 | 94.96 |
| | 18% DPPC + 2% | 50° C. | 1 Wk. | 81 | 80.92 |
| | NaCl + 0.075% EDTA | | | | |

Similar to the case for EDTA, poor results were also seen for the case of alpha tocopherol added at similar levels. Although the amount of chemical degradation after 1 week was less than that seen for EDTA, significant clump formation was seen for the powder after 2 weeks at 40 C, because of which the FPF<5.6 was observed to fall significantly as seen in Table 47. This study was discontinued due to this clumping.

TABLE 47
FPF <5.6 data for 15% Zolmitriptan produced
with varied a-tocopherol as an anti-oxidant

| | | | | FPF <5.6 | Purity |
|---|---|---|---|---|---|
| Lot # | Composition | Condition | | (%) | (%) |
| 214026 | 15% Zolmitriptan + | t = 0 | | 84 | NT |
| | 64.99% L-leu + 18% | 20° C. | 1 Wk. | NT | 99.84 |
| | DPPC + 2% NaCl + | | 2 Wk. | 85 | NT |
| | 0.01% α-Tocopherol | 40° C. | 1 Wk. | NT | 98.94 |
| | | | 2 Wk. | 49 | NT |
| 214027 | 15% Zolmitriptan + | t = 0 | | 80 | NT |
| | 64.925% L-leu + 18% | 20° C. | 1 Wk. | NT | 99.84 |
| | DPPC + 2% NaCl + | | 2 Wk. | 87 | NT |
| | 0.075% α-Tocopherol | 40° C. | 1 Wk. | NT | 98.87 |
| | | | 2 Wk. | 58 | NT |

### Example 15

### Evaluation with Addition of Food-Grade Oxygen Absorber Packs

As an alternative solution for reducing oxidation of Zolmitriptan in spray-dried powders, food grade oxygen absorbers were evaluated. Oxy-sorb 100 (Manufacturer: Dry Air Technologies, Tamil Nadu, India) was the initial selected oxygen absorber. Formulation A powders produced according to the spray-drying conditions utilized for Example 14 were filled into size 00 capsules (Quali-V, color HP OP White 8), packaged in Aluminum pouches with and without the selected oxygen absorbers and placed on stability at accelerated temperature conditions (40 and 50° C.). As shown in Table 48, samples exposed to this absorber were observed retain their high purity at both temperatures compared to controls. However, the powder was observed to turn extremely clumpy in the presence of the oxygen absorber and displayed a significant drop in the FPF<5.6 value after 1 week of storage. It appeared that the high water content of the initial absorbers selected facilitated a conversion of the amorphous zolmitriptan in the powders into a crystalline state, which likely acted to reduce the FPF<5.6 of said powders via inter-particle sintering.

TABLE 48
FPF <5.6 data for 15% Zolmitriptan produced with varied filling & packaging conditions

| | | | | | FPF <5.6 | Purity |
|---|---|---|---|---|---|---|
| Lot # | Description | | Condition | | (%) | (%) |
| 224001 | 15:65:18:2 | | t = 0 | | 89 | NT |
| | Zolmi:L- | No pH adj, No N2 purge, | 40° C. | 1 Wk. | 79 | 99.37 |
| | leu:DPPC:NaCl | No O2 packs, Filling = | | 2 Wk. | NT | 98.74 |
| | | 20 C., 15% RH | | 1 Mo. | 77 | 97.85 |
| | | | 50° C. | 1 Wk. | 49 | 98.42 |
| | | | | 2 Wk. | NT | 95.73 |
| | | | | 1 Mo. | 35 | 93.47 |
| | | No pH adj, No N2 purge, | 40° C. | 1 Wk. | 5 | 99.64 |
| | | Yes O2 packs, Filling = | | 2 Wk. | NT | NT |
| | | 20 C., 15% RH | | 1 Mo. | NT | NT |
| | | | 50° C. | 1 Wk. | 0 | 99.73 |
| | | | | 2 Wk. | NT | NT |
| | | | | 1 Mo. | NT | NT |
| | | No pH adj, With N2 purge, | 40° C. | 1 Wk. | 79 | 99.52 |
| | | No O2 packs, Filling = | | 2 Wk. | NT | 99.27 |
| | | 20 C., 0% RH | | 1 Mo. | 85 | 99.16 |
| | | | 50° C. | 1 Wk. | 64 | 99.11 |
| | | | | 2 Wk. | NT | 97.51 |
| | | | | 1 Mo. | 64 | 98.31 |

### Example 16

### Evaluation with Oxygen Absorber and Filling/Packaging Under an Inert Nitrogen Atmosphere

In addition to the use of oxygen absorbing materials, filling and packaging under an inert nitrogen atmosphere is another potential route to reduce the oxidation of zolmitriptan in dry powder formulations. To test this hypothesis, Formulation A powders spray-dried at 4 g/L utilizing the processing conditions described in Example 13 were filled and packaged under three conditions: (1) air at 15% RH, (2) a Nitrogen-purged glove box at 15% RH (with humidified Nitrogen gas), and a nitrogen purged glove box with 0% RH (with dry Nitrogen gas). Capsules filled under these conditions were placed on stability with and without Pharmakeep pouches. As seen in Table 49, all samples having the PharmaKeep pouches and those packaged at 0% RH under nitrogen were observed to have a consistently high FPF<5.6 and a high purity value (in excess of 99%) throughout the stability evaluation over 1 month at 40 C.

TABLE 49
FPF <5.6 and Purity data for 15% Zolmitriptan
produced with varied filling & packaging conditions
with and without O2 absorber (KD-20).

| | | | | FPF <5.6 | Purity |
|---|---|---|---|---|---|
| Lot # | Description | Condition | | (%) | (%) |
| 224061-1 | Filling = Room | 20° C. | 1 Mo. | 80 | NT |
| | (20 C., 15% RH), | 30° C. | 1 Mo. | 79 | NT |
| | Packaging = Room | 40° C. | 2 Wk. | 86 | 98.84 |
| | (20 C., 15% RH), | | 1 Mo. | 81 | 98.21 |
| | O2 abs + dess = | | | | |
| | No | | | | |
| 224061-2 | Filling = Room | 20° C. | 1 Mo. | 79 | NT |
| | (20 C., 15% RH), | 30° C. | 1 Mo. | 79 | NT |
| | Packaging = Room | 40° C. | 2 Wk. | 89 | 99.62 |
| | (20 C., 15% RH), | | 1 Mo. | 81 | 99.42 |
| | O2 abs + dess = | | | | |
| | Yes | | | | |
| 224061-3 | Filling = GloveB | 20° C. | 1 Mo. | 78 | NT |
| | (20 C., 15% RH), | 30° C. | 1 Mo. | 75 | NT |
| | Packaging = | 40° C. | 2 Wk. | 74 | 99.28 |
| | GloveB | | 1 Mo. | 78 | 98.26 |
| | (20 C., 15% RH), | | | | |
| | O2 abs + dess = | | | | |
| | No | | | | |
| 224061-4 | Filling = GloveB | 20° C. | 1 Mo. | 79 | NT |
| | (20 C., 15% RH), | 30° C. | 1 Mo. | 76 | NT |
| | Packaging = | 40° C. | 2 Wk. | 82 | 99.59 |
| | GloveB | | 1 Mo. | 76 | 99.35 |
| | (20 C., 15% RH), | | | | |
| | O2 abs + dess = | | | | |
| | Yes | | | | |
| 224061-5 | Filling = GloveB | 20° C. | 1 Mo. | 79 | NT |
| | (20 C., 0% RH), | 30° C. | 1 Mo. | 79 | NT |
| | Packaging = | 40° C. | 2 Wk. | 83 | 99.45 |
| | GloveB | | 1 Mo. | 78 | 99.19 |
| | (20 C., 0% RH), | | | | |
| | O2 abs + dess = | | | | |
| | No | | | | |
| 224061-6 | Filling = GloveB | 20° C. | 1 Mo. | 80 | NT |
| | (20 C., 0% RH), | 30° C. | 1 Mo. | 81 | NT |
| | Packaging = | 40° C. | 2 Wk. | 81 | 99.42 |
| | GloveB | | 1 Mo. | 78 | 99.41 |
| | (20 C., 0% RH), | | | | |
| | O2 abs + dess = | | | | |
| | No | | | | |

### Example 17

### Comparison of Leucine and SD-30 Based Formulations Under Oxygen Absorber/Nitrogen Filling/Packaging Conditions (Pharmakeep KD-20)

A similar evaluation to that described in Chemical Stability Example 4 was performed utilizing a Formulation A powder made with 65% SD-30, 18% DPPC and 2% NaCl. This evaluation was conducted by filling and packaging the SD-30 based powder in two conditions: (1) under air at 15% RH and (2) in a Nitrogen-purged glove box at 0% RH (with dry Nitrogen gas). Samples were spray dried at 12 g/L using process parameters listed in Table 50.

TABLE 50

| | | Value |
|---|---|---|
| Parameters | Total solid concentration (g/L) | 12 |
| | Inlet Temperature (° C.) | 103 |
| | Outlet Temperature (° C.) | 60 |
| | Drying Gas Rate (kg/hr) | 128 |
| | Atomization Gas Flow Rate (g/min) | 22 |
| | Aqueous Flow (mL/min) | 16 |
| | Organic flow (mL/min) | 24 |
| | Secondary drying gas flow (kg/hr) | 0 |
| | Secondary drying gas temperature (° C.) | 0 |
| | Product filter purge gas flow (scfh) | 15 |
| Atomization | Air cap | 67147 |
| nozzle | Fluid cap | 2850 |

Samples were filled and packaged under both conditions with and with the PharmaKeep KD-20 pouches. As seen in Table 51, all samples packaged with Pharmakeep KD-20 pouches and the sample that was packaged without the KD-20 pouch under dry nitrogen were observed to maintain consistently high FPF<5.6 and purity values throughout the stability evaluation over 3 months at 40° C.

TABLE 51
FPF <5.6 and Purity data for SD-30 based 15% Zolmitriptan produced
with varied filling & packaging conditions with and without KD-20.

| | | | | | | Impurities (%) | | | |
|---|---|---|---|---|---|---|---|---|---|
| | | | | FPF <5.6 | Purity | N- | Rel. | RRT ~0.54 | |
| Lot # | Description | Condition | | (%) | (%) | Oxide | A | or ~0.48 | Others |
| 214096 | Bulk powder | t = 0 | | 72 | 99.91 | 0.03 | 0.03 | 0 | 0.03 |
| 214096-1 | Fill/Pack: Dry | 40° C. | 2 Wk. | 70 | 99.87 | 0.03 | 0.03 | 0.02 | 0.05 |
| | Lab + KD-20 | | 1 Mo. | 78 | 99.86 | 0.03 | 0.02 | 0.05 | 0.03 |
| | (20 C., <15% | | 3 Mo. | 77 | NT | NT | NT | NT | NT |
| | RH under Air) | | | | | | | | |
| 214096-2 | Fill/Pack: Dry | 40° C. | 2 Wk. | 57 | 99.68 | 0.13 | 0.11 | 0 | 0.08 |
| | Lab + no KD-20 | | 1 Mo. | 66 | 99.35 | 0.27 | 0.22 | 0.02 | 0.14 |
| | (20 C., <15% | | 3 Mo. | 66 | NT | NT | NT | NT | NT |
| | RH under Air) | | | | | | | | |
| 214096-3 | Fill/Pack: Glove | 40° C. | 2 Wk. | 46 | 99.84 | 0.03 | 0.02 | 0.03 | 0.08 |
| | box + KD-20 | | 1 Mo. | 78 | 99.84 | 0.03 | 0.03 | 0.06 | 0.04 |
| | (20 C., <5% | | 3 Mo. | 77 | NT | NT | NT | NT | NT |
| | RH under N2) | | | | | | | | |
| 214096-4 | Fill/Pack: Glove | 40° C. | 2 Wk. | 69 | 99.9 | 0.03 | 0.03 | 0 | 0.04 |
| | box + no KD-20 | | 1 Mo. | 77 | 99.85 | 0.04 | 0.03 | 0.03 | 0.05 |
| | (20 C., <5% | | 3 Mo. | 74 | NT | NT | NT | NT | NT |
| | RH under N2) | | | | | | | | |

Formulation A was also produced utilizing the parameters shown in Table 50 and filled/packaged under similar conditions to those described for the SD-30 based formulation. As seen in Table 52, all samples packaged with Pharmakeep KD-20 pouches and the sample that was packaged without the KD-20 pouch under dry nitrogen were observed to maintain a consistently high purity value throughout the stability evaluation over 3 months at 40° C. Samples packaged with KD-20 pouches were also observed to maintain a consistently high FPF<5.6 value throughout the evaluation over 3 months at 40° C. The sample packaged without the KD-20 pouch under dry nitrogen at <5% RH inside the glove box shows a slight drop in FPF<5.6 value after 3 months at 40° C.

TABLE 52
FPF <5.6 and Purity data for Formulation Aproduced with
varied filling & packaging conditions with and without KD-20.

| | | | | | | Impurities (%) | | | |
|---|---|---|---|---|---|---|---|---|---|
| | | | | FPF <5.6 | Purity | N- | Rel. | RRT ~0.54 | |
| Lot # | Description | Condition | | (%) | (%) | Oxide | A | or ~0.48 | Others |
| 214087 | Bulk powder | t = 0 | | 74 | 99.92 | 0.03 | 0.03 | 0 | 0.03 |
| 214087-1 | Fill/Pack: Dry | 40° C. | 2 Wk. | 75 | 99.78 | 0.03 | 0.02 | 0.16 | 0.01 |
| | Lab + KD-20 | | 1 Mo. | 76 | 99.54 | 0.03 | 0.02 | 0.36 | 0.04 |
| | (20 C., <15% | | 3 Mo. | 62 | 98.79 | 0.02 | 0.01 | 1.13 | 0.05 |
| | RH under Air) | | | | | | | | |
| 214087-2 | Fill/Pack: Dry | 40° C. | 2 Wk. | 79 | 99.43 | 0.27 | 0.18 | 0.03 | 0.09 |
| | Lab + no KD-20 | | 1 Mo. | 60 | 98.96 | 0.49 | 0.32 | 0.05 | 0.18 |
| | (20 C., <15% | | 3 Mo. | 56 | 96.95 | 1.46 | 0.91 | 0.12 | 0.56 |
| | RH under Air) | | | | | | | | |
| 214087-3 | Fill/Pack: Glove | 40° C. | 2 Wk. | 76 | 99.74 | 0.03 | 0.04 | 0.18 | 0.01 |
| | box + KD-20 | | 1 Mo. | 75 | 99.52 | 0.02 | 0.02 | 0.39 | 0.05 |
| | (20 C., <5% | | 3 Mo. | 71 | 98.70 | 0.02 | 0.02 | 1.23 | 0.03 |
| | RH under N2) | | | | | | | | |
| 214087-4 | Fill/Pack: Glove | 40° C. | 2 Wk. | 76 | 99.84 | 0.04 | 0.02 | 0.09 | 0.01 |
| | box + no KD-20 | | 1 Mo. | 78 | 99.67 | 0.04 | 0.03 | 0.21 | 0.04 |
| | (20 C., <5% | | 3 Mo. | 61 | 99.31 | 0.06 | 0.03 | 0.54 | 0.06 |
| | RH under N2) | | | | | | | | |

A similar set of experiments was conducted with the zolmitriptan formulation utilizing a PharmaKeep CD-2.15G canister-type oxygen absorber and desiccator combo (Mitsubishi Gas Chemicals Company Inc., Tokyo, Japan). As seen in Table 53, all samples packaged with Pharmakeep CD-2.15G canisters and the sample that was packaged without the CD-2.15G canister under dry nitrogen were observed to maintain a consistently high purity value throughout the stability evaluation over 3 months at 40° C. Samples packaged with CD-2.15G canisters are also observed to maintain a consistently high FPF<5.6 value throughout the evaluation over 3 months at 40° C. The sample packaged without the CD-2.15G canister under dry nitrogen showed a slight drop in FPF<5.6 value after 3 months at 40° C.

TABLE 53
FPF <5.6 and Purity data for L-leu based 15% Zolmitriptan produced
with varied filling & packaging conditions with and without CD-2.15G

| | | | | | | Impurity (%) | | | |
|---|---|---|---|---|---|---|---|---|---|
| | | | | FPF <5.6 | Purity | N- | Rel. | RRT ~0.54 | |
| Lot # | Description | Condition | | (%) | (%) | Oxide | A | or ~0.48 | Others |
| 214104 | Bulk powder | t = 0 | | 76 | 99.86 | 0.04 | 0.06 | 0 | 0.04 |
| 214104-0 | Fill/Pack: Dry | 40° C. | 2 Wk. | 72 | 99.68 | 0.04 | 0.05 | 0.16 | 0.07 |
| | Lab + CD-2.15G | | 1 Mo. | 71 | 99.42 | 0.06 | 0.05 | 0.37 | 0.09 |
| | (Modified pouch) | | 3 Mo. | 67 | NT | NT | NT | NT | NT |
| | (20 C., <15% | | | | | | | | |
| | RH under Air) | | | | | | | | |
| 214104-1 | Fill/Pack: Dry | 40° C. | 2 Wk. | 71 | 99.71 | 0.04 | 0.04 | 0.17 | 0.04 |
| | Lab + CD-2.15G | | 1 Mo. | 74 | 99.34 | 0.07 | 0.06 | 0.41 | 0.12 |
| | (20 C., <15% | | 3 Mo. | 70 | NT | NT | NT | NT | NT |
| | RH under Air) | | | | | | | | |
| 214104-2 | Fill/Pack: Dry | 40° C. | 2 Wk. | 73 | 99.28 | 0.28 | 0.22 | 0.05 | 0.18 |
| | Lab + no CD-2.15G | | 1 Mo. | 67 | 98.79 | 0.51 | 0.33 | 0.12 | 0.25 |
| | (20 C., <15% | | 3 Mo. | NT | NT | NT | NT | NT | NT |
| | RH under Air) | | | | | | | | |
| 214104-3 | Fill/Pack: Glove | 40° C. | 2 Wk. | 72 | 99.62 | 0.05 | 0.05 | 0.2 | 0.08 |
| | box + CD-2.15G | | 1 Mo. | 76 | 99.39 | 0.05 | 0.04 | 0.43 | 0.09 |
| | (20 C., <5% | | 3 Mo. | 72 | NT | NT | NT | NT | NT |
| | RH under N2) | | | | | | | | |
| 214104-4 | Fill/Pack: Glove | 40° C. | 2 Wk. | 70 | 99.72 | 0.06 | 0.06 | 0.07 | 0.09 |
| | box + no CD-2.15G | | 1 Mo. | 72 | 99.54 | 0.1 | 0.07 | 0.17 | 0.12 |
| | (20 C., <5% | | 3 Mo. | NT | NT | NT | NT | NT | NT |
| | RH under N2) | | | | | | | | |

### Example 18

### Filling and Packaging Evaluation (GMP Versus PD)

This study was conducted evaluate the effect of varying the capsule filling and packaging configurations on the chemical stability, aerosol stability, and the solid state stability of L-leucine based powders produced using two different spray-drying setups: (1) PD NIRO PSD-1 system and (2) GMP Automatic PSD-1 system. Two PD bulk powder lots and one GMP bulk powder lot were produced and utilized for this stability evaluation: (i) PD Lot #224156 produced with Nitrogen purge on both aqueous and organic feed tanks, (ii) PD Lot #224152 produced without Nitrogen purge on both aqueous and organic feed tanks, and (iii) GMP lot B-2019-0003 also produced without Nitrogen purge on both aqueous and organic feed tanks. All three lots were spray dried at 12 g/L using process parameters listed in Table 50. All three powders were filled into clear Size 2 inhalation grade HPMC capsules (Qualicaps Quali-V-I), packaged into aluminum pouches and placed on stability. The different types of PharmaKeep absorbers include KD-20 canister-type which is oxygen and moisture absorbing, CD-2.15G canister-type which is oxygen and moisture absorbing, and CH-1G which is oxygen absorbing only (Mitsubishi Gas Chemicals Company Inc., Tokyo, Japan). Table 54 summarizes the feed tank nitrogen purge status of these runs, along with the production environment details.

TABLE 54
Spray drying, filling, and packaging
configuration of L-leu based powders

| | | | Nitrogen | | |
|---|---|---|---|---|---|
| | | Production | headspace | | |
| | Powder | area | Feed | Capsule | Filling |
| Lot number | filling area | environment | tanks | filling | setup |
| 224152 | PD room | 20° | N | N | Hand filling |
| | (solid | C., <15% | | Y | Hand filling |
| 224156 | state lab) | RH | Y | N | Hand filling |
| | | | | Y | Hand filling |
| B-2019-0003 | GMP suite | 20° | N | N | Hand filling |
| | (Rm 2123) | C., <15% | | Y | Hand filling |
| | | RH | | | |

Table 55 details FPF<5.6, gPSD and purity measurements for these lots. All lots displayed consistently high FPFs that were relatively stable over time. The lot produced under the GMP spray-drying setup also displayed excellent chemical stability over the 3 month course of the study.

TABLE 55
Aerosol and purity data for Spray drying, filling, and packaging configuration of L-leu based powders.

| Bulk | Packaging | N2 headspace | | | Filling | Packaging | Packaging | Lot | | | FPF <5.6 | Purity |
|---|---|---|---|---|---|---|---|---|---|---|---|---|
| powder | environment | Feed | Fill | Pack | setup | setup | aids | number | Conditions | | (%) | (%) |
| 224152 | Bulk powder | | | | | | | | t = 0 | | NT | 99.67 |
| (Zolmi + | PD Room | N | N | N | Hand | A1 pouch | CD-2.15G | 224152-1 | 40° C. | 1 Mo. | 69 | NT |
| Leu) | (Solid | | | | | | | | | 3 Mo. | 64 | NT |
| | state lab) | | | | | | CH-1G | 224152-2 | 40° C. | 1 Mo. | 59 | NT |
| | | | | | | | | | | 3 Mo. | 57 | NT |
| | | | | | | | n.d. | 224152-3 | 40° C. | 1 Mo. | 60 | NT |
| | | | | | | | | | | 3 Mo. | 54 | NT |
| | | | | Y | Hand | A1 pouch | n.d. | 224152-4 | 40° C. | 1 Mo. | 58 | NT |
| | | | | | | | | | | 3 Mo. | 56 | NT |
| | | | Y | Y | Hand | A1 pouch | n.d. | 224152-5 | 40° C. | 1 Mo. | 63 | NT |
| | | | | | | | | | | 3 Mo. | 57 | NT |
| 224156 | Bulk powder | | | | | | | | t = 0 | | 76 | 99.72 |
| (Zolmi + | PD Room | N | N | N | Hand | A1 pouch | CD-2.15G | 224156-1 | 40° C. | 1 Mo. | 71 | NT |
| Leu) | (Solid | | | | | | | | | 3 Mo. | 71 | NT |
| | state lab) | | | | | | CH-1G | 224156-2 | 40° C. | 1 Mo. | 63 | NT |
| | | | | | | | | | | 3 Mo. | 58 | NT |
| | | | | | | | n.d. | 224156-3 | 40° C. | 1 Mo. | 62 | 98.2 |
| | | | | | | | | | | 3 Mo. | 59 | NT |
| | | | | Y | Hand | A1 pouch | n.d. | 224156-4 | 40° C. | 1 Mo. | 63 | 98.52 |
| | | | | | | | | | | 3 Mo. | 59 | NT |
| | | | Y | Y | Hand | A1 pouch | n.d. | 224156-5 | 40° C. | 1 Mo. | 67 | 98.74 |
| | | | | | | | | | | 3 Mo. | 65 | NT |
| B-2019-0003 | Bulk powder | | | | | | | | t = 0 | | 66 | 99.75 |
| (Zolmi + | PD Room | N | N | N | Hand | A1 pouch | CD-2.15G | B-2019-0003-1 | 40° C. | 1 Mo. | 64 | NT |
| Leu) | (Solid | | | | | | | | | 3 Mo. | 68 | NT |
| | state lab) | | | | | | CH-1G | B-2019-0003-2 | 25 C. | 3 mth | NT | 99.91 |
| | | | | | | | | | 40° C. | 1 Mo. | 61 | 99.75 |
| | | | | | | | | | | 3 Mo. | 54 | 99.73 |
| | | | | | | | n.d. | B-2019-0003-3 | 40° C. | 1 Mo. | 57 | 98.35 |
| | | | | | | | | | | 3 Mo. | 56 | 96.83 |
| | | | | Y | Hand | A1 pouch | n.d. | B-2019-0003-6 | 40° C. | 1 Mo. | 58 | 98.71 |
| | | | | | | | | | | 3 Mo. | NT | NT |
| | | | Y | Y | Hand | A1 pouch | n.d. | B-2019-0003-9 | 40° C. | 1 Mo. | 66 | 99.72 |
| | | | | | | | | | | 3 Mo. | 64 | 99.42 |

### Example 19

### Evaluation of Filled Capsule Conditioning

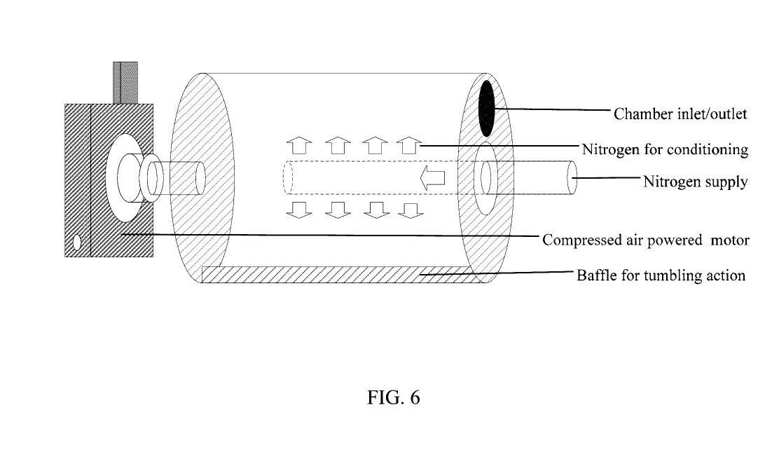

As another potential solution to reducing the potential for oxidation of zolmitriptan in packaged capsules, this study was conducted evaluate the effect of post-fill conditioning capsules that were filled in an air environment using a custom tumble drying apparatus. The tumbling apparatus that was used for this evaluation is shown in FIG. 6.

Two variations of this setup were investigated: (1) utilization of dry Nitrogen as the conditioning gas and (2) humidified Nitrogen. Tumbling was carried out in a glove box maintained at the target temperature/RH conditions. Both variations are described in the sub-sections below. Table 56 details FPF, gPSD and purity measurements obtained for case (1). Chemical purity over the course of the study remained high, indicating a good potential for the use of post-filling conditioning to reduce the potential for oxidative degradation of filled capsules.

TABLE 56
Aerosol and purity data for Filled capsule tumbling experiment with Dry N2

| | | | | | Impurities (%) | | |
|---|---|---|---|---|---|---|---|
| | | | | Purity | N- | Rel. | |
| Lot # | Description | Condition | | (%) | Oxide | A | Others |
| B-2019-0003 | Bulk powder | t = 0 | | 99.75 | 0.08 | 0.06 | 0.11 |
| 224199-1 | Before tumbling | t = 0 | | 99.87 | 0.05 | 0.04 | 0.04 |
| 224199-2 | Filled capsules after | t = 0 | | 99.88 | 0.04 | 0.04 | 0.04 |
| | tumbling for 4 hours | 40° C. | 1 Mo. | 99.60 | 0.08 | 0.05 | 0.27 |

Table 57 details FPF<5.6, gPSD and purity measurements for case (2). Chemical purity also remained high for this setup, with the use of humidified nitrogen reducing the potential for the capsules to become statically charged, which can limit their ability to be packaged.

TABLE 57
Aerosol and purity data for Filled capsule tumbling experiment with humidified N2.

| | | | | | | Impurities (%) | | |
|---|---|---|---|---|---|---|---|---|
| | | | | FPF <5.6 | Purity | N- | Rel. | |
| Lot # | Description | Condition | | (%) | (%) | Oxide | A | Others |
| 224152- | Tumbling with | t = 0 | | 68 | 99.75 | 0.08 | 0.14 | 0.03 |
| Tum | humidified N₂ | 40° C. | 2 Wk. | NT | 99.5 | 0.14 | 0.12 | 0.24 |
| | for 4 hours | | 1 Mo. | NT | 99.44 | 0.3 | 0.1 | 0.16 |

### Example 20

### Evaluation of Increased DPPC Fraction with Decreasing L-Leucine Fraction

This study was conducted to evaluate the effect of varying the DPPC:L-leucine ratio on the chemical stability, aerosol stability, and the solid state stability of L-leucine based powders produced using the PD Automatic PSD-1 systems. Three runs were executed for the purpose of this evaluation, and three DPPC:L-leu ratios were evaluated: (1) 65:18, (2) 45:38, and (3) 25:58. All three lots were spray dried at 12 g/L, and process parameters used for producing these lots were the same as those summarized in Table 50. All three powders were filled into clear Size 2 inhalation grade HPMC capsules (Qualicaps Quali-V-I), packaged into aluminum pouches and placed on stability. Table 58 details FPF, gPSD and purity measurements, and Table 559 details measurements of the solid state properties over the course of this stability study. Increasing the DPPC load was seen to have a negative effect on the FPF<5.6 of the resultant powders, further indicating the optimal properties of the 65:18 leucine:DPPC ration in the formulation containing 15% zolmitriptan.

TABLE 58
Aerosol and purity data for increased DPPC fraction SD runs.

| | | | | | | | Impurities (%) | | |
|---|---|---|---|---|---|---|---|---|---|
| | | | | gPSD | FPF <5.6 | Purity | N- | Rel. | |
| Lot # | Description | Condition | | (um) | (%) | (%) | Oxide | A | Others |
| 224182 | 65:18 | t = 0 | | 4.3 | 75 | 99.91 | 0.03 | 0.03 | 0.03 |
| | Leu:DPPC | 25° C. | 1 Mo. | NT | 73 | NT | NT | NT | NT |
| | | 40° C. | 1 Mo. | 4.9 | 59 | NT | NT | NT | NT |
| 224183 | 45:38 | t = 0 | | 4 | 71 | 99.86 | 0.04 | 0.04 | 0.06 |
| | Leu:-DPPC | 25° C. | 1 Mo. | NT | 65 | NT | NT | NT | NT |
| | | 40° C. | 1 Mo. | 5.3 | 50 | NT | NT | NT | NT |
| 224184 | 25:58 | t = 0 | | 5.2 | 49 | 99.86 | 0.05 | 0.03 | 0.06 |
| | Leu:DPPC | 25° C. | 1 Mo. | NT | 56 | NT | NT | NT | NT |
| | | 40° C. | 1 Mo. | 7.7 | 38 | NT | NT | NT | NT |

TABLE 59
Solid state data for increased DPPC fraction SD runs.

| | | | | Solid state | | | | | |
|---|---|---|---|---|---|---|---|---|---|
| | | | | | | | | Recryst. | |
| | | | | | TGA-120 | Low T 1 | Low T 2 | Onset | Recryst. |
| Lot # | Description | Condition | | XRPD | (%) | (° C.) | (° C.) | (° C.) | ΔH (J/g) |
| 224182 | 65:18 | t = 0 | | PC-T2 | 0.11 | 43.9 | 58.7, 69 | 120.1 | 13.30 |
| | Leu:DPPC | 40° C. | 1 Mo. | NT | 0.22 | 41.0 | 59.2, 69.6 | 130.3 | 10.4 |
| 224183 | 45:38 | t = 0 | | NT | 0.15 | 35.3 | 59.4, 69.6 | 119.5 | 9.98 |
| | Leu:DPPC | 40° C. | 1 Mo. | NT | 0.51 | 38.5 | 59.4, 69.8 | 115.6 | 11.8 |
| 224184 | 25:58 | t = 0 | | NT | 0.75 | 36.6 | 59, 70.1 | 114.7 | 7.56 |
| | Leu:DPPC | 40° C. | 1 Mo. | NT | 1.09 | 39.5 | 59.8, 70.1 | 117.7 | 8.3 |

### Example 21

### Clinical Study Examining the Safety and Pharmacokinetics of a Zolmitriptan Formulation

An open-label safety and pharmacokinetic (PK) study of single ascending doses of Formulation A (15% zolimtriptan, 65% L-leucine, 18% DPPC, 2% NaCl by weight of dry solids) in healthy adult subjects (NCT02609945). Formulation A was provided in capsules of 0.825 mg zolmitriptan (estimated to deliver 0.6 mg zolmitriptan to the lungs) and 3.0 mg zolmitriptan (estimated to deliver 2.4 mg zolmitriptan to the lungs). The dose levels administered were dose level (DL) 1=0.6 mg; DL 2=1.2 mg; DL 3=2.4 mg, and DL 4=4.8 mg zolmitriptan (estimated lung-delivered dose). Zomig™ Tablet 5 mg and Zomig™ Nasal Spray 5 mg were administered as reference drugs for comparative purposes. After a screening visit (Visit 1), subjects who met eligibility criteria were enrolled in the study and scheduled for admission to 1 of 2 study units for a stay of up to 20 days (Visit 2). All subjects completing the study underwent 6 dosing periods with single dose treatments, beginning with the reference treatments. A washout of at least 1 day was imposed between the reference treatments and at least 2 days between each inhaled dose.

Subjects were dosed in staggered fashion to allow time for review of safety data and better management of serial assessments. At each unit, men (Group 1) were dosed before women (Group 2). The sequence of treatments was as follows (also refer to the dosing schedule in Table 60):
Periods 1 and 2, Reference Drugs: Subjects received Zomig™ oral tablet in Period 1 and Zomig™ nasal spray in Period 2. Groups 1 and 2 were dosed 1 hour apart.
Periods 3 to 6, Formulation A: Beginning with DL 1 in Period 3, Group 1, the lead-in group of male subjects was dosed first, and safety data from this group, with a focus on cardiopulmonary safety, was reviewed in the afternoon so that the dose was tested in a limited number of subjects before exposing the balance of the subjects, with Group 2 dosed in a similar fashion the following day. Before advancing to the next dose level, a safety review of the 24 h data from all subjects was conducted.

The subsequent inhalation dose levels (DL 2, 3 and 4) were administered in ascending order after an observance of the safety and tolerability data to be adequate to allow dose escalation. In each period, subjects followed the same sequence of dosing of the lead-in subjects (Group 1), with a safety review in the afternoon, followed by dosing of the remaining subjects (Group 2) on the next day. Subjects were discharged from the clinic after all of the assessments at the 24-hour time point in Period 6 were completed. Study staff contacted subjects by phone approximately 1 week after discharge in order to assess the subjects' health status, marking the completion of the subject's participation in the study. There were originally 11 subjects in Group 1 and 10 subjects in Group 2. Two subjects from Group 1 discontinued after oral treatment (prior to inhalational dosing); the rest of the Group 1 subjects completed all Formulation A and reference dosing levels. Two subjects from Group 2 did not complete the study; one subject dropped out after the 1.2 mg Formulation A dosing level and one dropped out after the 2.4 mg Formulation A dosing level, both due to personal, non-treatment related reasons.

TABLE 60
Dosing Schedule

| Reference Drugs | | | | Ascending Sequence Inhalational Doses | | | | | |
|---|---|---|---|---|---|---|---|---|---|
| Dosing Period 1 | | Dosing Period 2 | | Dosing Period 3 | | | Dosing Period 4 | | |
| Oral | | Nasal | | Formulation A DL1 | | | Formulation A DL2 | | |
| Day 1 | Day 2 | Day 3 | Day 4 | Day 5 | Day 6 | Day 7 | Day 8 | Day 9 | Day 10 |
| Groups | | Groups | | Group | Group | Safety | Group | Group | Safety |
| 1&2 | | 1&2 | | 1 | 2 | Review | 1 | 2 | Review |

Ascending Sequence Inhalational Doses
(continued)

| | |
|---|---|
| Dosing Period 5 | Dosing Period 6 |
| Formulation A DL3 | Formulation A DL4 |

| Day 11 | Day 12 | Day 13 | Day 14 | Day 15 |
|---|---|---|---|---|
| Group | Group | Safety | Group | Group |
| 1 | 2 | Review | 1 | 2 |

In each dosing period, a total of 19 blood samples were collected from all subjects for pharmacokinetic assessments. Samples were collected within 15 minutes prior to dose administration and at 18 serial time points over a 24-hour period after the dose. With a minimum of 3 mL for each sample, the total blood drawn in the study for PK assessments was approximately 340 mL.

With respect to safety, research staff measured pulse oximetry, pulmonary function during inhalation dosing, and routine vital signs at serial time points. Continuous cardiac monitoring began 1 h prior to dosing and was performed for 6 h post-dose in each dosing period, and subjects were carefully monitored throughout the study for adverse events. In Periods 3-6, subjects were dosed one at a time, with at least a 1-hour interval between each subject receiving an inhalation dose to allow adequate time for assessment of post-dose safety. Safety reviews were conducted between Groups 1 and 2, and between each ascending dose level in order to maximize clinical and physiologic safety monitoring and to provide sufficient time to examine all subjects and review data before advancing or discharging from the unit.

### Pharmacokinetic Results

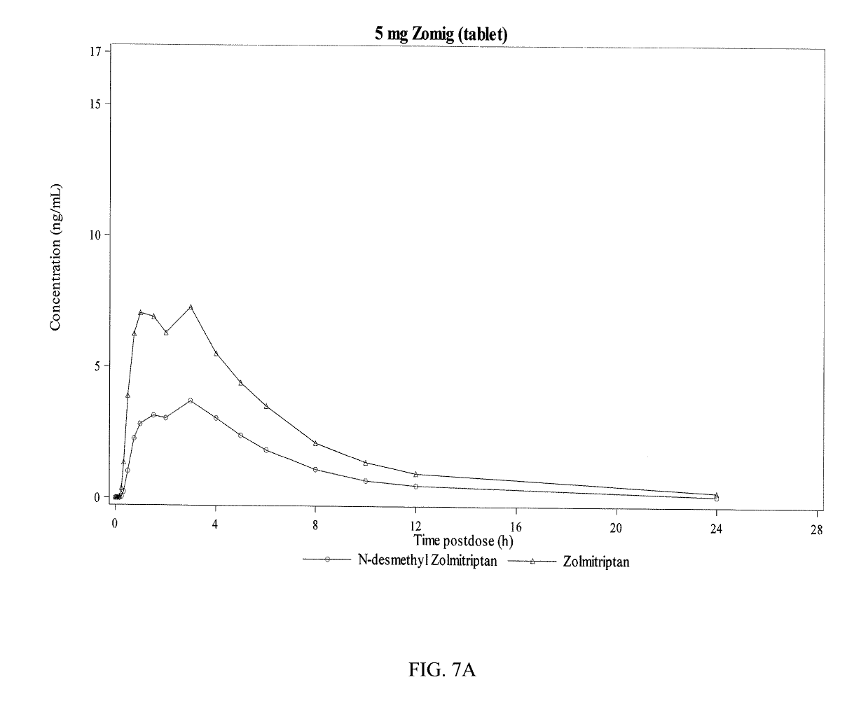

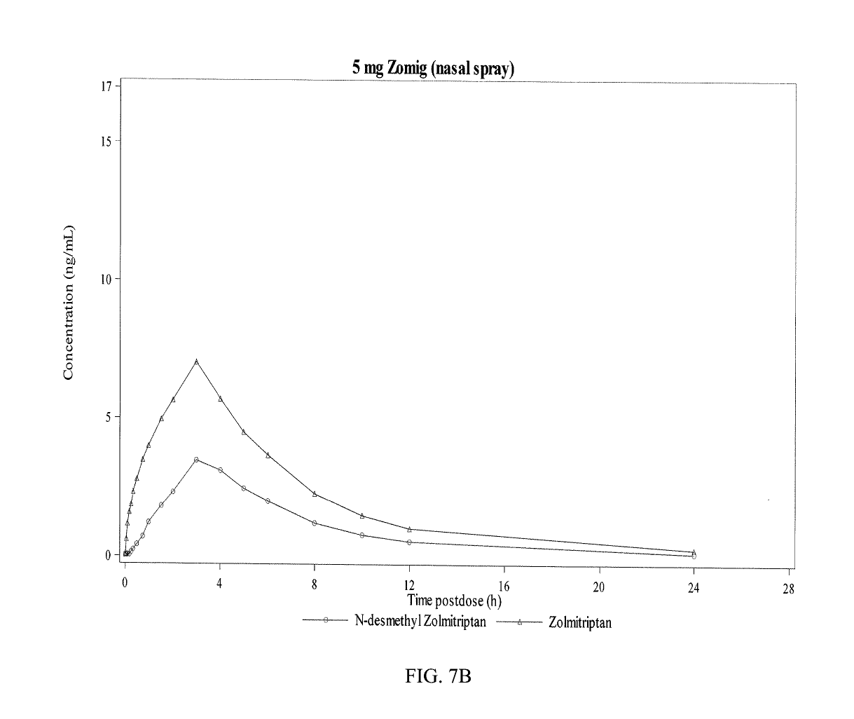

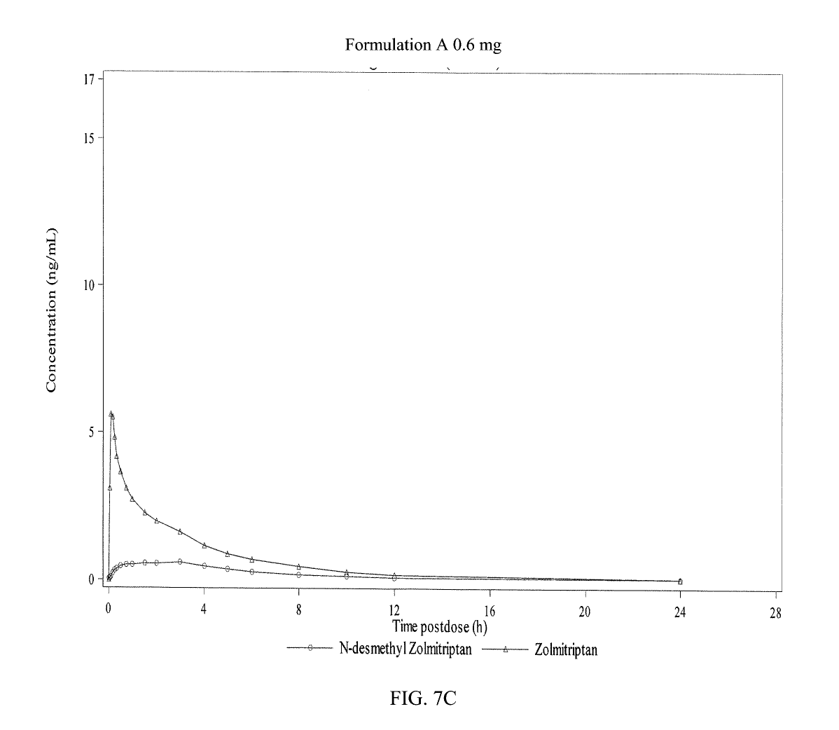

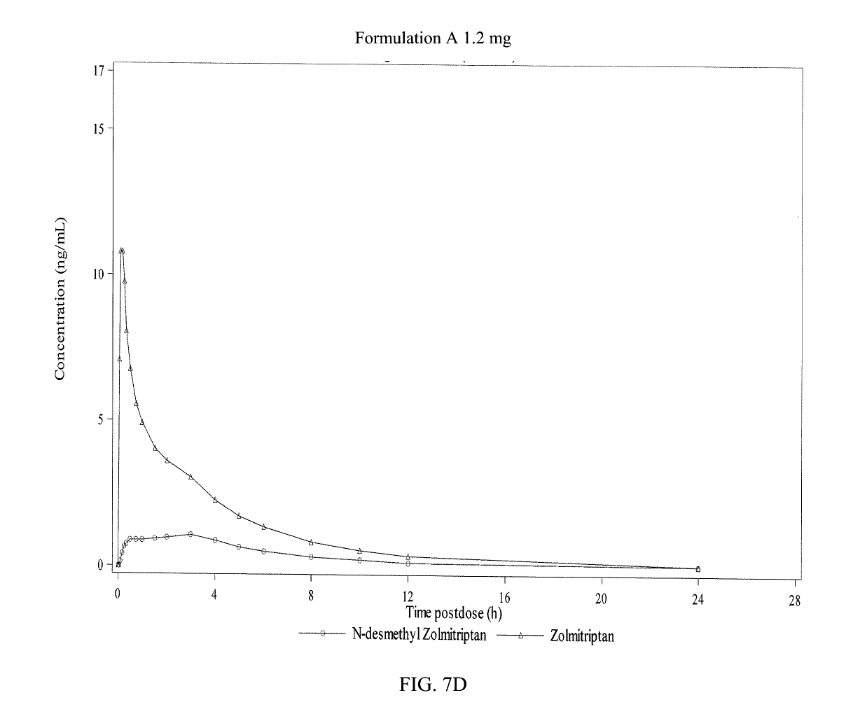

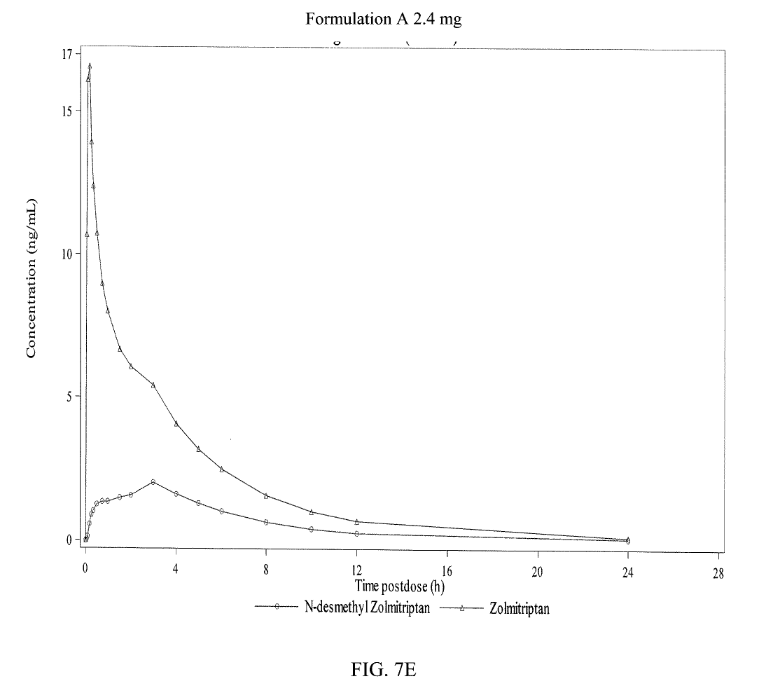

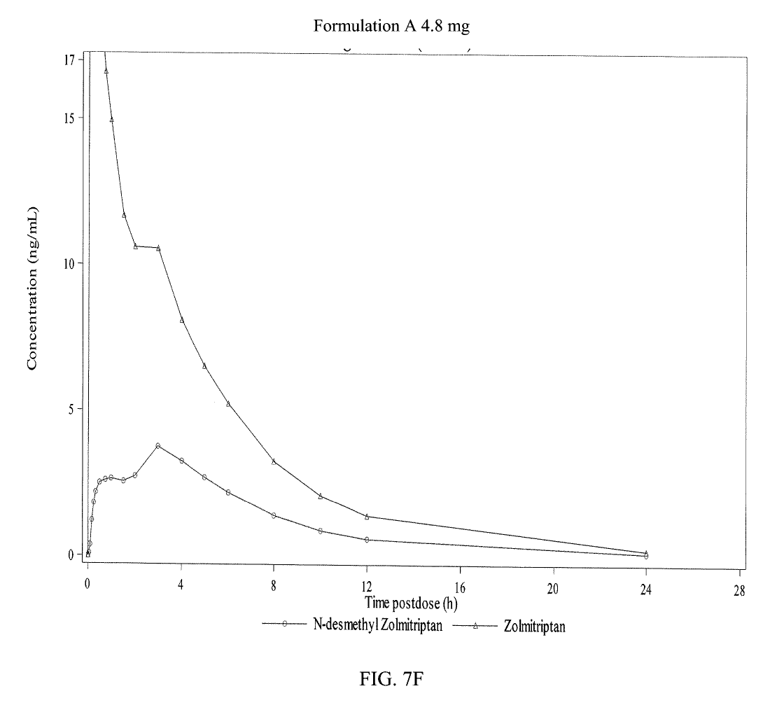

Pharmacokinetic results for each of the Formulation A and reference (oral and nasal) dosing groups are shown in FIGS. 7A-7F. As can be seen from the plots, administration of Formulation A resulted in a rapid and high uptake of zolmitriptan into the systemic circulation, with the 0.6 mg Formulation A dose resulting in a comparable Cₘₐₓ to the 5 mg oral and nasal doses with a much shorter tₘₐₓ. Higher doses of Formulation A mirrored this behavior with a dose-proportional increase in Cₘₐₓ. The relevant pharmacokinetic parameters are summarized in Table 61 (all data is expressed as mean data except for tₘₐₓ, which is presented as median data).

TABLE 61
Pharmacokinetic data

| | | Cₘₐₓ (CV) | tₘₐₓ (median) | t_{1/2} | AUC_{0-4 h} | AUC_{0-12 h} | AUC_{0-24 h} |
|---|---|---|---|---|---|---|---|
| | | (ng/mL) | (h) | (h) | (ng · h/mL) | (ng · h/mL) | (ng · h/mL) |
| Zolmitriptan | Oral | 8.7 (48.1) | 1.50 | 6.69 (29.5) | 20.2 (45.6) | 38.5 (38.4) | 49.0 (38.4) |
| | Nasal | 8.1 (34.0) | 3.00 | 6.04 (18.3) | 21.2 (36.8) | 43.6 (36.1) | 50.8 (37.6) |
| | 0.6 | 6.0 (35.7) | 0.167 | 5.01 (48.5) | 9.26 (26.6) | 13.5 (27.2) | 14.7 (29.9) |
| | 1.2 | 11.8 (34.9) | 0.167 | 4.77 (26.9) | 17.3 (28.1) | 25.2 (26.8) | 27.3 (27.2) |
| | 2.4 | 17.8 (33.1) | 0.167 | 4.83 (16.2) | 28.4 (27.6) | 43.1 (26.2) | 47.1 (26.7) |
| | 4.8 | 35.0 (36.6) | 0.167 | 4.64 (10.2) | 53.1 (29.1) | 83.1 (27.3) | 91.0 (27.5) |
| N-des-methyl | Oral | 4.04 (35.6) | 3.00 | 6.55 (26.2) | 10.6 (41.1) | 21.0 (26.9) | 24.4 (23.6) |
| zolmitriptan | Nasal | 3.89 (33.4) | 3.00 | 6.53 (15.5) | 8.93 (46.9) | 21.0 (33.0) | 24.9 (31.1) |
| | 0.6 | 0.66 (29.6) | 2.00 | 6.99 (41.0) | 2.06 (27.4) | 3.88 (23.8) | 4.71 (25.7) |
| | 1.2 | 1.16 (31.7) | 3.00 | 6.42 (21.5) | 3.63 (30.3) | 6.86 (23.2) | 8.00 (21.4) |
| | 2.4 | 2.08 (32.4) | 3.00 | 6.01 (16.6) | 6.14 (28.8) | 12.3 (21.5) | 14.2 (20.4) |
| | 4.8 | 3.85 (30.7) | 3.00 | 5.50 (12.3) | 11.4 (32.4) | 24.0 (22.3) | 28.0 (20.3) |
| Zolmitriptan | Oral | 2.83 (22.9) | 3.00 | 5.96 (39.4) | 7.33 (26.1) | 14.7 (21.5) | 16.8 (21.3) |
| N-oxide | Nasal | 2.71 (32.4) | 3.00 | 4.92 (17.8) | 6.10 (42.4) | 14.8 (35.8) | 17.2 (36.9) |
| | 0.6 | 0.45 (28.5) | 2.07 | 3.27 (39.2) | 1.44 (31.2) | 2.58 (32.1) | 2.82 (32.1) |
| | 1.2 | 0.83 (31.1) | 3.00 | 3.56 (32.3) | 2.66 (31.0) | 4.91 (27.2) | 5.38 (27.4) |
| | 2.4 | 1.49 (35.2) | 3.00 | 3.94 (24.9) | 4.46 (30.2) | 8.78 (28.8) | 9.80 (29.6) |
| | 4.8 | 2.69 (29.3) | 3.00 | 4.25 (12.1) | 8.20 (31.9) | 16.8 (28.1) | 18.9 (27.3) |

As can be seen from Table 58, administration of zolmitriptan via Formulation A resulted in significantly elevated Cₘₐₓ and reduced tₘₐₓ compared to the 5 mg oral and nasal reference doses. AUC values over the first 4 hours were also greater for the 2.4 and 4.8 mg doses of Formulation A compared to the reference products, with AUC values over the first 12 and 24 hours for the 2.4 mg Formulation A dose being comparable to the 5 mg doses of the reference products. Additionally, despite the rapidity of uptake to the systemic circulation that is comparable to s.c. or i.v. injection, the data from Table 58 indicates that the half-life for the pK of zolmitriptan via inhalation of Formulation A is only slightly less than that seen for the oral and nasal forms (approximately 5 hours for inhalation versus 6 hours for nasal and 7 hours for oral). This is comparable to the reported half-lives of s.c. zolmitriptan or sumatriptan, which are typically on the order of 2 hours. This may indicate that Formulation A possesses the dual advantage of having a fast onset and systemic uptake that is comparable to s.c. yet has a duration of effect approaching oral.

Administration of zolmitriptan by inhalation of Formulation A also surprisingly appears to be much less variable as compared to either oral or nasal administration, with the variability (as represented by the % CV) of parameters such as Cₘₐₓ and AUC being approximately 30% (similar to that typically seen following s.c. or i.v. injection of triptans) versus approaching 50% for oral delivery (the CV values for tₘₐₓ for Formulation A are relatively high due to the surprisingly small values obtained for tₘₐₓ for Formulation A). With respect to inhalation of Formulation A versus oral delivery of tablets containing zolmitriptan, the pulmonary route advantageously avoids several of the phenomena that can lead to a high degree of intra- and inter-subject variability from oral administration. This includes avoidance of first-pass metabolism in addition to other factors that can act to increase the variability of oral drug administration, such as variability in gastric motility and gastric emptying, administration in a fed versus fasted state, etc. Thus, the reduced degree of variability in absorption and transport to the systemic circulation combined with the rapid uptake provides Formulation A with significant advantages versus Zomig™ oral tablets. Similar advantages of Formulation A administered via pulmonary inhalation likely also exist with respect to nasal delivery, which is also known to be variable and is often times seen to not possess any advantages with respect to speed of onset versus oral administration.

The rapidity and completeness of delivery of zolmitriptan to the systemic circulation via administration of Formulation A can also be evidenced via several additional pharmacokinetic measures as summarized in Table 59. As can be seen in Table 62, the dose-normalized (DN) Cₘₐₓ for Formulation A was approximately 4-6× greater than that seen for the oral and nasal formulations. The percent AUC seen over the first hour compared to the AUC over the 24 hour sampling period for each of the doses of Formulation A was approximately 25% as compared to approximately 6-7% for oral or nasal delivery, with a similar trend evident with respect to the AUC over the first 2 hours of dosing. This indicates that delivery of zolmitriptan via Formulation A results in a much more rapid entry of zolmitriptan into the systemic circulation via pulmonary delivery by Formulation A as compared to either oral or nasal delivery. Finally, the dose-normalized AUC over 24 hours for Formulation A was approximately 2× that of the reference products, indicating approximately a 2× increase in bioavailability of zolmitriptan via inhalation of Formulation A as compared to oral or nasal delivery of zolmitriptan. Given the reported value of 40% for the oral bioavailability of zolmitriptan, these results indicate a bioavailability approaching 100% for zolmitriptan via administration from Formulation A, further indicating the efficiency of delivery of the invention disclosed herein.

TABLE 62

| | DN- | AUC(0- | AUC(0- | DN-AUC(0- |
|---|---|---|---|---|
| | Cmax | 1 hr)/ | 2 hr)/ | 24 hr) |
| | (ng/ | AUC(0- | AUC(0- | (ng · |
| | mL/mg) | 24 hr) | 24 hr) | hr/ml) |
| Oral (5 mg) | 1.74 | 6.9% | 19.9% | 9.81 |
| Nasal (5 mg) | 1.62 | 5.6% | 15.5% | 10.2 |
| Formulation | 9.93 | 25.4% | 41.2% | 24.5 |
| A 0.6 mg | | | | |
| Formulation | 9.83 | 26.0% | 41.0% | 22.8 |
| A 1.2 mg | | | | |
| Formulation | 7.41 | 23.6% | 37.8% | 19.6 |
| A 2.4 mg | | | | |
| Formulation | 7.29 | 23.2% | 36.4% | 19.0 |
| A 4.8 mg | | | | |

An additional novel and surprising result of the invention disclosed herein is the differences seen in the metabolism of zolmitriptan to both the active metabolite (N-desmethyl zolmitriptan) and major inactive (zolmitriptan N-oxide) forms of zolmitriptan. As can be seen from FIG. 6 and Table 58, the conversion of zolmitriptan to the active and inactive metabolite forms occurs at a much slower rate upon administration of Formulation A than seen upon administration of the oral and nasal reference products, likely due in part to the avoidance of first-pass metabolism via inhalation. This potentially allows for an optimization of the efficacy and adverse event profile of the present invention versus conventional oral and nasal dosage forms of zolmitriptan. It is also interesting to note that the half-life of the major active metabolite N-desmethyl zolmitriptan is comparable for Formulation A and the reference products (approximately 6 hours). Formulation A may thus potentially possess the combined advantages of a fast onset as well as an extended duration of action, thus combining the best features of injectable (fast onset) and oral delivery (more extended duration of action) of triptans.

Finally, it is also surprising and advantageous that pulmonary delivery of zolmitriptan via Formulation A may possess significant advantages with respect to tolerability and patient acceptance versus nasal delivery. The nasal delivery of zolmitriptan and other triptans such as sumatriptan is known to be associated with a bad taste as reported by patients, with this bad taste lingering for minutes or hours following nasal administration due to such phenomenon as post-nasal drip, etc. The subjects in our trial did not provide any indication of a bad taste resulting from the inhalation of Formulation A. The high FPFs seen for the zolmitriptan formulations disclosed herein indicates that very little of the dose deposits in the oropharyngeal cavity, with the majority of the dose efficiently reaching the lungs. This potentially translates into a greatly reduced potential for the occurrence of a bad taste upon administration of Formulation A in addition to a reduced potential for a lingering bad taste, as none of the Formulation A dose is expected to deposit in the nasal cavity.

Thus, administration of zolmitriptan by inhalation via the invention disclosed herein possesses several significant advantages over the delivery of zolmitriptan via the oral or nasal route. First, administration of Formulation A results in a high and rapid uptake of zolmitriptan into the systemic circulation that appears to mimic the administration of zolmitriptan via s.c. or i.v. administration. This rapid delivery has the potential to allow for fast relief of migraine symptoms compared to administration of zolmitriptan via the oral or nasal route. In addition, Formulation A also appears to have a pharmacokinetic profile that indicates that the rapidity of uptake will not compromise the duration of action of Formulation A as compared to oral or nasal delivery, with the half-lives of both zolmitriptan and its major active metabolite being comparable to those seen for oral and nasal delivery. Formulation A thus has the surprising and advantageous potential for providing for relief of migraine symptoms that is both rapid and robust.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A powder formulation for pulmonary delivery to the respiratory tract of a patient, wherein the powder particles comprise zolmitriptan or a pharmaceutically acceptable salt thereof, and one or more excipients selected from a phospholipid, a salt, an amino acid, a sugar and a sugar alcohol.

2. The powder formulation of claim 1, comprising zolmitriptan free base.

3. The powder formulation of claim 2, wherein the particles comprise zolmitriptan in amorphous form.

4. The powder formulation of claim 2, wherein the particles comprise zolmitriptan in crystalline form.

5. The powder formulation of claim 2, wherein the particles comprise zolmitriptan in both crystalline and amorphous form.

6. The powder formulation of claim 2, wherein the zolmitriptan is in an amorphous form and at least one excipient is in a crystalline or partially crystalline form.

7. The powder formulation of claim 1, wherein the powder has a fine particle fraction<5.6 of at least about 60%.

8. The powder formulation of claim 7, wherein the fine particle fraction<5.6 is at least about 70%.

9. The powder formulation of claim 8, wherein the fine particle fraction<5.6 is from about 70% to about 90%.

10. The powder formulation of claim 2, wherein the powder is from about 5 to about 30% zolmitriptan by dry weight.

11. The powder formulation of claim 10, wherein the particles are from about 10 to about 20% zolmitriptan by dry weight.

12. The powder formulation of claim 11, wherein the particles are about 15% zolmitriptan by dry weight.

13. The powder formulation of claim 1, wherein the particles comprise zolmitriptan free base, a phospholipid, a salt and an additional excipient selected from amino acids, sugars and sugar alcohols.

14. The powder formulation of claim 13, wherein the phospholipid is selected from the group consisting of dipalmitoylphosphatidylcholine, dilauroylphosphatidylcholine, and disaturated-phosphatidylcholine, or a combination of two or more thereof.

15. The powder formulation of claim 14, wherein the phospholipid is dipalmitoylphosphatidylcholine.

16. The powder formulation of claim 1, wherein the salt is sodium chloride, sodium citrate, sodium lactate or potassium chloride.

17. The powder formulation of claim 16, wherein the salt is sodium chloride.

18. The powder formulation of claim 1, wherein the particles comprise a hydrophobic amino acid.

19. The powder formulation of claim 18, wherein the amino acid is selected from the group consisting of leucine, isoleucine, alanine, valine, phenylalanine and glycine.

20. The powder formulation of claim 19, wherein the amino acid is L-leucine.

21. The powder formulation of claim 1, wherein the particles comprise a sugar or sugar alcohol selected from the group consisting of maltodextrin, polyglycitol, lactose, trehalose and mannitol.

22. The powder formulation of claim 21, wherein the sugar is maltodextrin or polyglycitol.

23. The powder formulation of claim 1, wherein the particles comprise zolmitriptan, dipalmitoylphosphatidylcholine, sodium chloride and maltodextrin.

24. The powder formulation of claim 1, wherein the particles comprise zolmitriptan, dipalmitoylphosphatidylcholine, sodium chloride and L-leucine.

25. The powder formulation of claim 24, wherein the zolmitriptan is present in the particles in an amorphous form and the L-leucine is present in the particles in a crystalline or partially crystalline form.

26. The powder formulation of claim 1, wherein the particles comprise zolmitriptan, dipalmitoylphosphatidylcholine, sodium chloride and polyglycitol.

27. The powder formulation of claim 26, wherein the zolmitriptan is present in the particles in an amorphous form and the dipalmitoylphosphatidylcholine is present in the particles in a crystalline or partially crystalline form.

28. The powder formulation of claim 1, wherein the particles comprise zolmitriptan, dipalmitoylphosphatidylcholine, sodium chloride, L-leucine and polyglycitol.

29. The powder formulation of claim 1, wherein said powder consists essentially of particles comprising about 5 to about 50% zolmitriptan, about 5 to about 20% phospholipid, and about 1 to about 10% salt as measured by weight percent of dry solids in the powder.

30. The powder formulation of claim 1, wherein said powder consists essentially of particles comprising about 5 to about 30% zolmitriptan, about 5 to about 20% phospholipid, about 1 to about 10% salt and about 50 to 80% sugar, sugar alcohol or amino acid as measured by weight percent of dry solids in the powder.

31. The powder formulation of claim 1, wherein said powder consists essentially of particles comprising about 10 to about 25% zolmitriptan, about 5 to about 20% dipalmitoylphosphatidylcholine, about 1 to about 10% sodium chloride or sodium citrate and about 50 to about 80% polyglycitol or L-leucine as measured by weight percent of dry solids in the powder.

32. The powder formulation of claim 31, wherein the particles comprise L-leucine and the zolmitriptan is present in the particles in an amorphous form and the L-leucine is present in the particles in a crystalline or partially crystalline form.

33. The powder formulation of claim 31, wherein the particles comprise polyglycitol, zolmitriptan is present in the particles in an amorphous form and the dipalmitoylphosphatidylcholine is present in the particles in a crystalline or partially crystalline form.

34. The powder formulation of claim 1, wherein said powder consists essentially of particles comprising about 15% zolmitriptan, about 2% salt, about 18% phospholipid and about 65% sugar, sugar alcohol or amino acid as measured by weight percent of dry solids in the powder.

35. The powder formulation of claim 34, wherein said particles comprise about 65% L-leucine as measured by weight percent of dry solids in the powder.

36. The powder formulation of claim 35, wherein the L-leucine is present in the particles in a crystalline or partially crystalline form.

37. The powder formulation of claim 34, wherein the particles comprise about 65% polyglycitol as measured by weight percent of dry solids in the powder.

38. The powder formulation of claim 34, wherein the phospholipid is dipalmitoylphosphatidylcholine and the salt is sodium chloride or sodium citrate.

39. The powder formulation of claim 38, wherein the salt is sodium chloride.

40. A powder formulation for pulmonary delivery to the respiratory tract of a patient, said powder consisting essentially of particles having a composition selected from the table below, as measured by weight percent of dry solids in the powder:

| Zolmi- | | Sodium | Maltodextrin | L- | |
|---|---|---|---|---|---|
| triptan | DPPC | Chloride | (DE = 10.7%) | Leucine | Polyglycitol |
| 25% | 18% | 2% | 55% | 0 | 0 |
| 10% | 18% | 2% | 70% | 0 | 0 |
| 10% | 18% | 2% | 0 | 0 | 70% |
| 10% | 8% | 2% | 0 | 0 | 80% |
| 20% | 8% | 2% | 0 | 0 | 70% |
| 20% | 18% | 2% | 0 | 0 | 60% |
| 10% | 18% | 2% | 0 | 70% | 0 |
| 10% | 8% | 2% | 0 | 80% | 0 |
| 20% | 8% | 2% | 0 | 70% | 0 |
| 20% | 18% | 2% | 0 | 60% | 0 |
| 15% | 18% | 2% | 0 | 0 | 65% |
| 15% | 18% | 2% | 0 | 65% | 0 |

41. The powder formulation of claim 1, wherein pulmonary administration of said powder formulation to an adult human subject exhibits a tₘₐₓ of about 8 to 10 minutes.

42. The powder formulation of claim 41, wherein administration of the powder formulation at a zolmitriptan dose of 2.4 mg to the subject's lungs provides a Cₘₐₓ of about 10 to about 40 ng/mL.

43. The powder formulation of claim 42, wherein the Cₘₐₓ is about 10 to about 20 ng/mL.

44. The powder formulation of claim 42, wherein said administration provides an AUC₀₋₂₄ for zolmitriptan of about 30 to about 60 ng*hr/mL.

45. The powder formulation of claim 44, wherein said administration provides an AUC₀₋₂₄ for zolmitriptan of about 40 to about 50 ng*hr/mL.

46. A method of delivering zolmitriptan to the pulmonary system of a patient comprising the steps of:
a) providing a powder formulation of claim 1 in a compartment and an inhaler to a patient;
b) dispersing the powder by breath actuation of the patient; and
c) delivering the powder particles to the patient's respiratory system.

47. A method of treating migraine in a subject in need thereof, comprising administering to the pulmonary system of the subject a therapeutically effective amount of the powder formulation of claim 1.

48. A method of treating cluster headache in a subject in need thereof, comprising administering to the pulmonary system of the subject a therapeutically effective amount of the powder formulation of claim 1.
